(19) United States
(12) Patent Application Publication
(10) Pub. No.: US 2008/0163824 A1
(43) Pub. Date: Jul. 10, 2008

(54) Whole genome based genetic evaluation and selection process

(75) Inventors: Gerhard Christian Moser, Bellbowrie (AU); Herman W. Raadsma, Werombi (AU); Bruce Tier, Armidale (AU); Alexander Frederick Woolaston, Armidale (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Innovative Dairy Products Pty Ltd, an Australian company, ACN 098 382 784, Melbourne (AU)

(21) Appl. No.: 11/849,134

(22) Filed: Aug. 31, 2007

Related U.S. Application Data
(60) Provisional application No. 60/841,898, filed on Sep. 1, 2006.
(60) Provisional application No. 60/919,178, filed on Mar. 20, 2007.

(30) Foreign Application Priority Data
Mar. 15, 2007 (AU) 2007/901355
Mar. 20, 2007 (AU) 2007/901501

(51) Int. Cl. A01K 67/02; G06G 7/60

(52) U.S. Cl. 119/174; 703/11

(57) ABSTRACT

The present invention provides a method and system for the prediction of the merit of at least one individual in a population, the method comprising the steps of: (a) in the population, where information of individuals are known, using dimension reduction on the information to project the information to a low dimensional space whilst retaining the complexity of the information to generate a set of explanatory variables; (b) utilising the explanatory variables to generate a predictor function with respect to merit; and (c) utilising the predictor function to predict the merit of the individual.

### CROSS-REFERENCE TO RELATED APPLICATIONS

The present application is a nonprovisional and claims the benefit of U.S. of America Provisional Application No. 60/841,898, filed on Sep. 1, 2006, and U.S. of America Provisional Application No. 60/919,178, filed Mar. 20, 2007, both incorporated by reference in their entirety for all purposes. The present application also claims the benefit of Australian Provisional Application No. 2007901355, filed on Mar. 15, 2007, and Australian Provisional Application No. 2007901501, filed on Mar. 20, 2007, both incorporated by reference in their entirety for all purposes.

### TECHNICAL FIELD

Disclosed herein are methods for predicting genetic and phenotypic merit in individuals on the basis of genome-wide marker information. Also disclosed are methods for determining the fitness or predisposition of an individual for a desired purpose, or the susceptibility of the individual to an outcome, such as a disease. It should be recognized that the invention has a broad range of applicability.

### BACKGROUND

All references, including any patents or patent applications, cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents forms part of the common general knowledge in the art, in Australia or in any other country.

Genetic progress, for example in a herd, flock, group, crop, etc, depends on choices made as to the best individuals to use as breeding stock, on the basis of predictions of the superior performance of offspring yet to be born. The basis of such predictions is generally an estimate of genetic merit on the basis of the use of statistical analysis of performance or phenotypic data of an individual and that of its relatives where the data are analysed using statistical approaches such as best linear unbiased prediction (BLUP). This is a well-accepted procedure, and is the basis of genetic improvement schemes for several species of livestock in a number of countries. For example, such schemes have been used for dairy cattle in Australia, New Zealand, Canada and Holland, for sheep in Australia, New Zealand and the United Kingdom, and for poultry and pigs in a number of countries.

Although phenotypic measurements of a biological or performance trait can be recorded for an individual within a population, there is little or no useful phenotypic information available until the individual enters the productive phase of its life, which is normally adulthood. In the case of the dairy cow, this is its first lactation; for meat-producing animals such as beef cattle, pigs and sheep, it is harvesting, i.e. slaughter; for racing animals, it is when the animal commences training or actual racing. In the pre-production phase predictions of genetic merit for an individual rely entirely on the data on relatives of that individual. This lack of information on individuals within a population at an early stage reduces the ability to make decisions about the potential future use of such individuals especially with respect to their use in breeding. Consequently the rate of genetic gain in the biological or performance trait of the population under selection is less than that which would be achievable with such data.

Some performance traits are expressed in only one sex; such traits are known as sex-limited traits, with one example being milk production. However, the genetic merit of the sire for any heritable trait is very important in achieving genetic progress, in that an individual inherits around one-half of its genotype from each parent. Therefore it is advantageous to assess the genetic merit of an individual sire in order to define its value for breeding the next generation of progeny/descendants. This has led to progeny testing of young sires, which are then generally selected on the basis of Estimated Breeding Value (EBV), which is an estimate of their genetic merit.

In many commercially-important species, artificial breeding techniques such as artificial insemination (AI), in vitro fertilization (IVF), embryo transfer and the like are permissible and practicable. In such species, following progeny testing, the semen of the best (proven) sires is then made available for use in the wider population by artificial insemination (AI). Even though progeny testing delays the use of sires in the wider population, the cost-benefit is sufficiently great that artificial breeding companies invest a considerable amount in progeny testing each year. For example, the cost of progeny testing per young dairy or beef bull is around SA20,000 per head, and depending on the size of the company it is not uncommon for first year team size to be around 150 bulls.

The use of quantitative genetics in individual breeding programs is a powerful and important tool. For example, it has been a major driver of profitability and international competitiveness within the dairy industry in Australia and other countries. However, until recently the use of large-scale gene-marker technology to identify premium individuals and favourable traits has been immature, cumbersome and expensive. Some preliminary attempts at genome-wide analysis of data for dairy cattle have been described in artificial simulated data sets where both marker spacing and genetic (or so called Quantitative trait loci, QTL) effects were known and do not reflect naturally complex biological systems (Meuwissen et al, 2001; Gianola et al 2006). Furthermore in these studies the number and density of markers was relatively low compared to the quantity of genotypic data now becoming available which could contain a full genome sequence of each individual thus exacerbating problems which are overcome by this invention. Despite these limitations the hypothetical and yet as unproven advantages of using extensive marker information are highly prospective in both livestock (Schaffer, 2006) and plants (Bernardo and Yu, 2007) once again in artificial simulated un natural populations. Also, examples of attempts to apply neural network and genetic algorithms approaches to determine a variety of predictive applications based upon gene-hunting techniques to determine particular genes responsible for determining the desired outcome and is not applicable to a whole genomic approach to the situation. Therefore, despite previous attempts at gene analysis for predictive capabilities and the availability of genomic information for many species, the methods have hitherto not been widely applied because of difficulties in predicting correlation between gene markers such as single nucleotide polymorphisms (SNPs) and beneficial phenotypic traits. Even with the availability of validated SNPs or other markers and high-throughput genotyping methods, there is no generally accepted methodology for analysis of genotype data at the whole genome level.

Therefore, an improved system and method for analysing genotype data is desired.

### SUMMARY

The inventors have now devised a method for estimation of breeding values and phenotypic performance from SNP data, in which genome-wide variation in the SNP data is used to account for the variation in breeding values of phenotype by integrating dimension reduction and SNP selection to reduce the number of dimensions in the original SNP data and optimize model selection fort maximum predictive accuracy (i.e. minimal prediction error). In one arrangement, using this method enables the breeding value of an individual to be predicted without knowing the actual location of the SNP in the genome, and without having knowledge of the pedigree of the individual. Knowledge of the pedigree is helpful, but is not essential to the method. Also, knowledge of marker locations for a particular trait may also be helpful, but again are not necessary for the prediction of merit using the present method(s).

The presently described methods and systems disclosed herein cover aspects in gene marker and trait analyses and building predictive diagnostic tools. A process of dimension reduction is used that preserves the information in fewer dimensions without loss of information and without explicit modeling relationships between genotype and phenotype. This is achieved but not limited by use of PLS, PCA and SVM combined with optional cross validation. Furthermore the prediction equations derived may use a subset of markers which capture a large proportion of the original information. This is accomplished by combining dimension reduction and marker selection. Furthermore, the prediction equations (i.e. predictor function(s)) and marker selection may be derived by using a genetic algorithm or similar method.

The use of extensive genome wide genetic marker technologies allows many 1000's if not soon millions of markers to be measured in an individual. It is forecast that it will be technically possible to obtain the whole genome sequence for individuals at a reasonable price in the next decade. However, now and in the foreseeable future, in most cases many more marker observations are present than individuals measured (i.e. 50 to 500 million marker observations in 1000 individuals are common data structures). This presents the following problems in that not all markers can be explicitly fitted thus rendering usual methods for marker subset selection such as ordinary regression methods (stepwise, least angle regression) or QTL screening methods useless. Furthermore there are many 1000's of model combinations possible (theoretically an exponential increase in model combinations over the number of markers tested different models being fitted to the data where the total number of possible models is SUM(k=1 to N)N!/((N−k)!k!, the total number of specific models is SUM (k=1 to n_{data}) N!/((N−k)!k!, as fitting more than d SNP is redundant). Furthermore the close relationship between multiple markers in linkage disequilibrium means that many alternate markers may be used to account for the same trait-marker relationship therefore making finite model selection to maximise prediction of merit almost impossible. The ambiguity in interpretation of multiple marker models arises as a consequence of collinearity between the explanatory variables). Finally, the addition of multiple isolated genetic effects in conventional QTL mapping solutions or marker associations, present problems in accurately predicting total genetic merit, since each effect is subject to error and the sum total of all effects may be grossly over estimated thus limiting prediction and utility of high density marker applications in diagnostic applications of human, plant and animal. This invention describes means to handle all these problems in an integrated and systematic manner to maximize ascertainment of predictive functions between genome-wide marker information and merit in populations to which the marker information applies.

The methods disclosed herein demonstrate that a subset of markers may be used to explain a large proportion of the variation in a given trait in a population. The methods of the invention enable the identification of the minimum number of SNPs which explains the maximum variation of a trait. This can be established using the “training set” described herein. The selected set of SNPs is then used on the population of interest. The method can be used to design a panel, e.g. of SNPs, for each trait in a desired set of traits. It is expected that there may be some redundancy between the sets of SNPs for different traits.

According to an arrangement of a first aspect there is provided a method for the prediction of the merit of at least one individual in a population, the method comprising the steps of:

(a) in the population, where information of individuals are known, using dimension reduction on the information to project the information to a low dimensional space whilst retaining the complexity of the information to generate a set of explanatory variables;

(b) utilising the explanatory variables to generate a predictor function with respect to merit; and

(c) utilising the predictor function to predict the merit of the individual.

According to another arrangement of the first aspect, there is provided a method for a prediction of a merit of at least one individual, the method comprising the steps of:

(a) in a first population, where genotype and phenotype information of individuals in the first population are known, using dimension reduction on the genotype and phenotype information to determine the complexity of the genotype and phenotype information to minimise prediction error for at least one marker in the first population and thereby generate a set of explanatory variables with respect to the at least one marker;

(b) utilising the explanatory variables to the first population to generate predictor function with respect to merit;

(c) generating a genotype for the at least one marker in at least one individual of interest from a second population; and

(d) utilising the predictor function and the genotype of the at least one individual of interest to determine the genetic merit of the individual of interest with respect to the at least one marker.

According to a further arrangement of the first aspect, there is provided a method for the prediction of the merit of at least one individual in a population, the method comprising the steps of:

(a) in the population, where information of individuals are known, using a genetic algorithm process on the information to generate a set of explanatory variables for all the information, the explanatory variables comprising weighted averages for components of the information; and

(b) utilising the explanatory variables to generate a predictor function with respect to merit;

(c) utilising the predictor function to predict the merit of the individual

In any one of the arrangements of the first aspect, step (b) may comprise utilising the explanatory variables to generate a plurality of predictor functions for the individuals of the population. The information may comprises information for at least one marker. The information may comprise information for a plurality of marker s.

In any one of the arrangements of the first aspect, or in any arrangement of the following aspects, the information may be selected from the group of genotype, phenotype or genotype and phenotype information on individuals in the population, For a plurality of individuals of interest from the population where information is unknown, the method may further comprise generating genotype for at least one individual of interest from population.

In still further arrangements, the method may further comprise the steps of:

(f) determining additional information on the explanatory variables for the at least one individual;

(g) combining the additional information for the at least one individual with the information on the explanatory variables for the individuals of the population; and

(h) repeating steps (b) and (c) for at least one further individual to predict the merit of the further individual.

Step (f) may comprises determining additional information on the explanatory variables on a plurality of individuals.

In any one of the arrangements, the utilisation of the predictor function may be performed on the basis of a desired outcome.

The genotype information may comprises genetic markers or bio-markers or epigenetic markers.

The merit may be a genetic merit selected from the group of a molecular breeding value, a quantitative trait locus, or a quantitative trait nucleotide.

The sampling in step (a) may be random or it may be targeted. The targeted sampling may comprise sampling the first population on the basis of an outcome of interest.

Step (b) of the method may comprise defining a plurality of predictors for the sampled individuals of the first population. Step (c) may comprise determining the genotype for a plurality of markers. Step (c) may comprise determining the genotype for a plurality of individuals of interest.

The genotype may comprise genetic markers, bio-markers and/or epigenetic markers. The merit may be in the form of genetic merit. The genetic merit may be one or more of a molecular breeding value, the isolation and/or identification of a quantitative trait locus (QTL), a quantitative trait nucleotide (QTN), or other genotypic information. The merit may alternatively be in the form of the fitness of the individual of interest for a desired outcome. The merit may also be in the form of a diagnosis of a condition or susceptibility to a condition in the individual of interest.

The prediction of merit of the individual may involve only genotypes available for at least one of the predictor functions.

According to a second aspect there is provided a method for predicting trait performance for at least one individual of interest, the method comprising the steps of:

(a) in the population, where information of individuals are known, using dimension reduction on the information to project the information to a low dimensional space whilst retaining the complexity of the information to generate a set of explanatory variables; and

(b) utilising the explanatory variables to generate a predictor function with respect to merit;

(c) utilising the predictor function to predict the trait performance for the individual.

The method may further comprise the steps of:

(d) for an individual of interest from the population where information is unknown, generating genotype for at least one individual of interest from population; and

(e) applying the predictor function to the genotype of the at least one individual of interest to predict the predict the trait performance for the individual.

According to a third aspect there is provided a method for selecting at least one individual of interest, wherein said method comprises:

a) in a first population, where genotype and phenotype information of individuals in the first population are known, using dimension reduction on the genotype and phenotype information to determine the complexity of the genotype and phenotype information to minimise prediction error for at least one marker in the first population and thereby generate a set of explanatory variables with respect to the at least one marker;

(b) applying the explanatory variables to the first population to generate a predictor function;

(c) generating genotype for the at least one marker in at least one individual of interest from a second population;

(d) applying the predictor function to the genotype of the at least one individual of interest to select the individual.

According to a fourth aspect there is provided a method of diagnosing a condition in at least one individual of interest in a population, the method comprising the steps of:

(a) in the population, where information of individuals are known, using dimension reduction on the information to project the information to a low dimensional space whilst retaining the complexity of the information to generate a set of explanatory variables; and

(b) utilising the explanatory variables to generate a predictor function;

(c) utilising the predictor function to diagnose a condition in the individual

The method of diagnosing may further comprise the steps of

(d) for an individual of interest from the population where information is unknown, generating genotype for at least one individual of interest from population; and

(e) applying the predictor function to the genotype of the at least one individual of interest to diagnose a condition in the individual of interest.

The method includes drawing an inference regarding a trait of the subject for the health condition, from a nucleic acid sample of the subject. The inference is drawn by identifying at least one nucleotide occurrence of a SNP in the nucleic acid sample, wherein the nucleotide occurrence is associated with the trait

According to a fifth aspect, there is provided a method of prediction of a susceptibility to an outcome of at least one individual of interest in a population, the method comprising the steps of:

(a) in the population, where information of individuals are known, using dimension reduction on the information to project the information to a low dimensional space whilst retaining the complexity of the information to generate a set of explanatory variables; and

(b) utilising the explanatory variables to generate a predictor function;

(c) utilising the predictor function to predict the susceptibility of the individual to an outcome.

The prediction of a susceptibility to an outcome may further comprising the steps of:

(d) for an individual of interest from the population where information is unknown, generating genotype for at least one individual of interest from population; and

(e) applying the predictor function to the genotype of the at least one individual of interest to predict the susceptibility of the individual to an outcome

The outcome may be the susceptibility of the individual of interest to a disease. The outcome may be the susceptibility of the individual of interest to a response to a stimulus. The stimulus may be selected from the group of a medicament, toxin, or an environmental condition. The environmental condition may comprise water shortage, feed shortage, stress, sunlight, or other environmental condition.

According to a sixth aspect, there is provided a method of breeding at least one individual in a population, the method comprising the steps of:

(a) in the population, where information of individuals are known, using dimension reduction on the information to project the information to a low dimensional space whilst retaining the complexity of the information to generate a set of explanatory variables; and

(b) utilising the explanatory variables to generate a predictor function with respect to merit of the individual;

(c) utilising the predictor function to predict the merit of the individual and

(d) breeding from the individual of interest on the basis of the merit of the individual.

The method of breeding may further comprise the steps of:

(f) determining information for the descendants of the at least one individual;

(g) correlating the information for the descendants of the at least one individual to the predictor function; and

(h) selecting descendants of said individual on the basis of the relationship between the information for the descendants and the predictor function.

It will be appreciated that while methods of breeding cannot ethically be utilized with humans, there are situations in which a couple may be at significantly increased risk of having a child which suffers from a genetically-determined disease or condition. For example, genetic counseling is widely used to help couples to decide whether to have children or to proceed with a pregnancy. However, few conditions are determined by a single gene, and unless a relative of one of the couple is known to have a genetically-determined disease or condition, the couple may not be aware that there is any risk. This aspect of the invention is applicable to determination of risk and assisting a couple to arrive at an informed decision in the context of genetic counseling.

According to a seventh aspect there is provided a system for the prediction of merit of an individual in a population, the system comprising:

(a) in the population, where information of individuals are known, means for using dimension reduction on the information to project the information to a low dimensional space whilst retaining the complexity of the information to generate a set of explanatory variables; and

(b) means for utilising the explanatory variables to generate a predictor function with respect to merit;

(c) means for utilising the predictor function to predict the merit of the individual

1. According to an eighth aspect there is provided a system for predicting trait performance of at least one individual in a population, the system comprising;

a) in the population, where information of individuals are known, means for using dimension reduction on the information to project the information to a low dimensional space whilst retaining the complexity of the information to generate a set of explanatory variables; and

(b) means for utilising the explanatory variables to generate a predictor function; and

(c) means for utilising the predictor function to predict performance of said trait for the individual of interest.

The trait may be a quantitative trait.

According to a ninth aspect there is provided a system for selecting at least one individual in a population, the system comprising;

a) in the population, where information of individuals are known, means for using dimension reduction on the information to project the information to a low dimensional space whilst retaining the complexity of the information to generate a set of explanatory variables; and

(b) means for utilising the explanatory variables to generate a predictor function; and

(c) means for utilising the predictor function to select the individual.

According to an tenth aspect, there is provided a system for diagnosing a condition in at least one individual of interest in a population, the system comprising:

(a) in the population, where information of individuals are known, means for using dimension reduction on the information to project the information to a low dimensional space whilst retaining the complexity of the information to generate a set of explanatory variables; and

(b) means for utilising the explanatory variables to generate a predictor function;

(c) means for utilising the predictor function to diagnose a condition in the individual.

According to an eleventh aspect there is provided a system for prediction of a susceptibility to an outcome of at least one individual of interest in a population, the system comprising:

(a) in the population, where information of individuals are known, means for using dimension reduction on the information to project the information to a low dimensional space whilst retaining the complexity of the information to generate a set of explanatory variables; and

(b) means for utilising the explanatory variables to generate a predictor function;

(c) means for utilising the predictor function to predict the susceptibility of the at least one individual of interest to an outcome.

According to a twelfth aspect there is provided a system for breeding at least one individual in a population, the system comprising:

(a) in the population, where information of individuals are known, means for using dimension reduction on the information to project the information to a low dimensional space whilst retaining the complexity of the information to generate a set of explanatory variables; and

(b) means for utilising the explanatory variables to generate a predictor function with respect to merit of the individual;

(c) means for utilising the predictor function to predict the merit of the individual and

(d) means for breeding from the individual of interest on the basis of the merit of the individual.

The system may further comprise the steps of:

(f) means for determining information for the descendants of the at least one individual;

(g) means for correlating the information for the descendants of the at least one individual to the predictor function; and

(h) means for selecting descendants of said individual on the basis of the relationship between the information for the descendants and the predictor function.

In the fourth and tenth aspects, the diagnosis may be diagnosis of a disease or condition. For example, the disease may be any disease which affects productivity, performance or fertility. For example in dairy cattle these include metabolic disorder, mastitis, and wasting. The condition may be resistance to disease or infection, or susceptibility to infection with and shedding of pathogens such as E. coli, Salmonella species, Listeria monocytogenes, prions and other organisms potentially pathogenic to humans, regulation of immune status and response to antigens, susceptibility to conditions such as bloat, Johne's disease, or liver abscess, previous exposure to infection or parasites, or other health or respiratory and digestive problems.

In the fifth and eleventh aspects, the susceptibility may be susceptibility to a disease or condition. For example, the disease may be a metabolic disorder, mastitis, or wasting.

According to any one of the first to twelfth aspects, the information may comprise genetic information consisting essentially of marker genotypes. The genetic markers may be distributed substantially across the genome. The number of genetic markers genotyped may be greater than 1000, greater than 1500, greater than 2500, greater than 5000, greater than 10000, greater than 15000, greater than 20000, greater than 25000, greater than 30000, greater than 35000, greater than 40000, greater than 45000, greater than 50000, greater than 100000, greater than 250000, greater than 500000, or greater than 1000000, greater than 5000000, greater than 10000000 or greater than 15000000.

The genetic markers may be selected from the group consisting of single nucleotide polymorphism (SNP), tag SNP, microsatellite (simple tandem repeat STR, simple sequence repeat SSR), restriction fragment length polymorphism (RFLP), amplified fragment length polymorphism (AFLP), insertion-deletion polymorphism (INDEL), random amplified polymorphic DNA (RAPD), ligase chain reaction, insertion/deletions and direct sequencing of the gene or a simple sequence conformation polymorphisms (SSCP). The genetic marker may be a SNP.

The information may comprise at least one of the pedigree of the individual; an estimated breeding value of the individual; data on genetic markers across the genome for the individual or for relatives of the individual; at least one index of phenotype for the individual or for relatives of the individual; at least one marker predictive of phenotype for the individual or for relatives of the individual; and at least one index of epigenetic modification or status for the individual, or a combination thereof.

The individual may be a dairy cow or bull, and the quantitative trait may be selected from the group consisting of APR, ASI, protein kg, protein percent, milk yield, fat kg, fat percent, overall type, mammary system, stature, udder texture, bone quality, angularity, muzzle width, body depth, chest width, pin set, pin sign, foot angle, set sign, rear leg view, udder depth, fore attachment, rear attachment height, rear attachment width, centre ligament, teat placement, teat length, loin strength, milking speed, temperament, like-ability, survival, calving ease, somatic cell count, cow fertility, and gestation length, or a combination of one or more of these traits.

The dimension reduction may be selected from the a technique in the group consisting of principal component analysis (PCA), a genetic algorithm, a neural network, partial least squares (PLS), inverse least squares, kernel PCA, LLE, Hessian LLE, Laplacian Eigenmaps, LTSA, isomap, maximum variance unfolding, Bolzman machines, projection pursuit, a hidden Markov model support vector machines, kernel regression, discriminant analysis and classification, k-nearest-neighbour analysis, fuzzy neural networks, Bayesian networks, or cluster analysis.

The dimension reduction technique may be principal component analysis. The dimension reduction technique may be supervised principal component analysis. The number of principal components in the principle component analysis may be between about 10 and about 40. The number of principal components may be about 20.

The dimension reduction technique may be partial least squares analysis. The number of latent components in the partial least squares analysis may be between about 4 and about 10. The number of latent components may be about 6.

The dimension reduction technique may be support vector machine analysis.

In any one of the above aspects the information may not include the pedigree of the individual.

In one form of the above aspects, the training population is a subset of the test population. It is from these individuals that the relationships between the marker variants and the trait variation is ultimately established. The genotypes of other individuals can be determined for subsets and used with the predictor functions to determine any type of merit of those individuals.

The information may comprise either genotypic or phenotypic information, or a combination thereof, for the individuals in the population. The at least one individual may or may not have corresponding explanatory variables.

The information may comprise one, two, three or more of: the pedigree of the individual; an estimated breeding value of the individual; data on genetic markers across the genome for the individual or for one or more of its relatives; at least one index of phenotype for the individual or for one or more of its; at least one bio-marker predictive of phenotype for the individual or for one or more of its relatives; at least one index of epigenetic modification or status for the individual, and any other information which is indicative of, or potentially indicative of, genetic differences between individuals in the population, or a combination thereof. For example, other important explanatory variables for phenotypes may include any systematic effects which affect the data, such as age, age of dam, management group, herd, year, season, sex, maternal effects (genetic and environmental), and treatments of the animal, such as vaccination. At the phenotypic level comparison can only be made of ‘like’ with ‘like’.

The prediction of merit, the process of selection or the process of breeding for at least one individual, and systems involving same, may involve a predictor function or functions. The predictor functions may be genetic predictors, and may be derived from genetic markers, phenotypic information or other genetic information such as pedigree, correlated EBVs, genetic parameters such as heritabilities, variances and correlations, or a combination thereof. However, in some arrangements, the pedigree and or map locations (with respect to marker positions of a particular trait) may not be required for the prediction of merit.

The markers may be genetic markers, and may be selected from, but are not restricted to, the group consisting of single nucleotide polymorphism (SNP), tag SNPs, haplotype, microsatellite (simple tandem repeat STR, simple sequence repeat SSR), restriction fragment length polymorphism (RFLP), amplified fragment length polymorphism (AFLP), insertion-deletion polymorphism (INDEL), random amplified polymorphic DNA (RAPD), ligase chain reaction, insertion/deletion and direct sequencing of the gene or a simple sequence conformation polymorphism (SSCP). For example, the genetic marker may be a single nucleotide polymorphism (SNP). The markers may be distributed substantially across the genome.

The predictors are chosen using a dimension reduction technique. The dimension reduction technique may be selected from a variety of methods, including, but not limited to, principal component analysis (PCA), genetic algorithms, neural networks, partial least squares (PLS), inverse least squares, kernel PCA, locally linear embedding such as LLE, Hessian LLE, Laplacian Eigenmaps, LTSA), Isomap, Maximum Variance Unfolding, Bolzman machines, projection pursuit, a hidden Markov model support vector machines, kernel regression, discriminant analysis and classification, k-nearest-neighbour analysis, fuzzy neural networks, Bayesian networks, cluster analysis or other known dimension reductions techniques or may be a combination of a number of dimension reduction techniques for example partial least squares reduction in combination with a genetic algorithm process. Other examples are also listed in “A survey of dimension reduction techniques” (US DOE Office of Scientific and Technical Information, 2002). The dimension reduction technique may be a supervised dimension reduction technique such as supervised partial least squares analysis or supervised principle component analysis among others. Different methods give similar results, but vary in speed of computation. Neural networks and genetic algorithms are methods for reducing dimensions, and thus they could be used either directly or indirectly. For example PCA will transform 15000 SNP into N principal components, where N is the number of individuals; a genetic algorithm or a neural network could be used to choose among the principal components.

The dimension reduction technique may be partial least squares analysis. The dimension reduction technique may be logistic partial least squares analysis. The dimension reduction technique may be generalised partial least squares analysis. In other arrangements, the dimension reduction technique may be selected from the group of principal component analysis (PCA), neural networks, or projection pursuit.

The dimension reduction technique may be principal component analysis, and the number of principal components may be selected using a genetic algorithm, wherein the principal components may form the inputs to the genetic algorithm. In one embodiment the dimension reduction technique is supervised principal component analysis. The number of principal components is less than the number of data points. In one embodiment the number of principal components is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. The number of principal components may be about 20. The trait may be any quantitative trait. The trait may relate to any aspect relating to the group consisting of agricultural, livestock, performance and aquaculture animals, and plants used in agriculture, agronomy, forestry and horticulture.

It is understood that the methods described herein may be applied to any species for which both genomic information and phenotypic information is available. Genomic information can include DNA sequences and data relating to single nucleotide polymorphisms (SNPs), haplotypes, and the like. Phenotypic information can include performance data, for example for dairy or beef cattle, sheep produced for wool or meat, or for animals used for racing. Phenotypic data also includes information regarding morbidity and disease susceptibility. As a result of the various genome projects, genomic data such as SNPs, haplotypes etc. are widely available. In addition to the human genome, partial or complete genome maps have been published for mammals, including chimpanzee, cattle, horse, dog, chicken, rat, mouse, Rhesus macaque, cat, other vertebrates, including zebrafish, medakafish, blowfish, and African clawed toad, and plants, including rice, wheat, maize, tomato, loblolly pine, and poplar. Some sequence data are also available for crustaceans such as shrimp; see for example U.S. Pat. No. 5,712,091.

Information about genome projects and links to their databases can be found on the World Wide Web, for example at the National Center for Biotechnology Information (www.ncbi.nlm.nih.gov/Genomes/index.html), which includes the databases for Online Mendelian Inheritance in Man (www.ncbi.nlm.nih.gov/Omim/) and the International HapMap Project (www.hapmap.org). The Genomes OnLine database (www.genomesonline.org) and the Institute for Genomic Research (www.tigr.org/tdb).

Performance data for livestock animals such as dairy cattle have been extensively recorded in countries such as Australia, Canada, New Zealand and Holland; similar data are available for beef cattle, pigs, chickens, and sheep. Performance data for thoroughbred racehorses, quarterhorses, standardbred trotting horses and pacers, endurance horses and Arab horses are available, in the case of thoroughbreds going back well over 100 years.

Thus the invention is particularly applicable to, but not limited to, the following types of individual:

a) Cattle: dairy and beef breeds;

b) Horses: racing breeds, e.g. thoroughbreds, standardbreds, quarterhorses, endurance horses, and Arabs;

c) Sheep: wool, meat and milk breeds;

d) Other fibre, meat and milk-producing animals, such as goats, alpacas, vicunas and llamas;

e) Other racing animals, such as camels;

f) Poultry, such as chickens, turkeys, geese and ducks;

g) Fish: farmed genera or species such as salmonids, including salmon, ocean trout, and freshwater trout; barramundi, tilapia and carp;

h) Crustaceans: farmed genera or species, such as prawns and shrimp;

i) Humans: prediction of sporting performance, especially for athletics events involving running and/or endurance, swimming, rowing and kayaking, and football codes (e.g. Australian Rules Football, rugby, American football, soccer), baseball, basketball and ice hockey; identification of markers useful in diagnosis of disease, estimation of risk of multifactorial genetic disorders; and identification of pharmacogenomic markers.

j) Plants: genera or species used in agriculture (crop or pasture), forestry or horticulture.

The quantitative trait may be one or more traits associated with dairy production, which may be selected from, but is not restricted to, the group consisting of Australian Profit Ranking (APR), ASI, protein kg, protein percent, milk yield, fat kg, fat percent, overall type, mammary system, stature, udder texture, bone quality, angularity, muzzle width, body depth, chest width, pin set, pin sign, foot angle, set sign, rear leg view, udder depth, fore attachment, rear attachment height, rear attachment width, centre ligament, teat placement, teat length, loin strength, milking speed, temperament, like-ability, survival, calving ease, somatic cell count, cow fertility, and gestation length, or a combination thereof. Any trait which is under genetic control in part and for which there is genetic variability can be used.

According to a thirteenth aspect there is provided a breeders product comprising at least one gamete with a high prediction of merit for at least one marker, the breeders product selected by a method for the prediction of the merit of at least one individual, the method comprising the steps of:

(a) in a first population, where genotype and phenotype information of individuals in the first population are known, using dimension reduction on the genotype and phenotype information to determine the complexity of the genotype and phenotype information to minimise prediction error for at least one marker in the first population and thereby generate a set of explanatory variables with respect to the at least one marker;

(b) applying the explanatory variables to the first population to generate a predictor function;

(c) generating genotype for the at least one marker in at least one individual of interest from a second population;

(d) applying the predictor function to the genotype of the at least one individual of interest to determine the genetic merit of the individual of interest with respect to the at least one marker.

According to a fourteenth aspect there is provided a computer system comprising a computer processor and memory, the memory comprising software code stored therein for execution by the computer processor of a method for the prediction of the merit of at least one individual in a population, the method comprising the steps of:

(a) in a database comprising information about the population, where information of individuals are known, using dimension reduction on the information to project the information to a low dimensional space whilst retaining the complexity of the information to generate a set of explanatory variables;

(b) utilising the explanatory variables to generate a predictor function with respect to merit; and

(c) utilising the predictor function to predict the merit of the individual.

In a fifteenth aspect there is provided a computer readable medium, having a program recorded thereon, where the program is configured to make a computer execute a procedure for the prediction of the merit of at least one individual in a population, the software product comprising:

(a) in a database comprising information about the population, where information of individuals are known, code for using dimension reduction on the information to project the information to a low dimensional space whilst retaining the complexity of the information to generate a set of explanatory variables;

(b) code for utilising the explanatory variables to generate a predictor function with respect to merit; and

(c) code for utilising the predictor function to predict the merit of the individual.

According to a eighteenth aspect, there is provided an information database product comprising information for individuals of a population, the information database for use with a method for the selection of at least one individual in the population, the method comprising the steps of:

(a) in the population, where information of individuals are known, using dimension reduction on the information to project the information to a low dimensional space whilst retaining the complexity of the information to generate a set of explanatory variables; and

(b) utilising the explanatory variables to generate a predictor function with respect to merit;

(c) utilising the predictor function to predict the merit of the individual.

According to a nineteenth aspect, there is provided an information database product for use with a breeding program, the database comprising information for individuals of a population and a prediction of the merit of the individuals in the population.

The individuals of interest from the population may be selected for use in a breeding program based upon the prediction of merit for the at least one marker.

According to a twentieth aspect, there is provided an information database product for use with a breeding program, the database comprising information for individuals of a population and a prediction of the merit of the individuals in the population.

The prediction of a merit of the individuals in the population is provided by a dimension reduction method on the genotype and phenotype information of individuals in the population comprising the steps of:

(a) using a dimension reduction method, determining the complexity of genotype and phenotype information of individuals in the population to minimise prediction error and thereby generate a set of explanatory variables;

(b) applying the explanatory variables to the first population to generate a predictor function;

(c) generating genotype for the at least one marker in at least one individual of interest from a second population;

(d) applying the predictor function to the genotype of the individuals of the second population thereby to determine the genetic merit of individuals in the second population individuals with respect to the at least one marker

Individuals of interest from the population may be selected for use in a breeding program based upon the prediction of merit for the at least one marker.

A system or method as claimed in any of the preceding claims wherein the predictor function is a predictor function with having minimal prediction error

The method of any one or more of the first to twelfth aspects may be implemented using a computer system 1000, such as that shown in FIG. 15 wherein the processes of FIGS. 1A to 1D may be implemented as software, such as one or more application programs executable within the computer system 1000. FIG. 15 is merely an example, which should not unduly limit the scope of the claims. One of ordinary skill in the art would recognize many variations, alternatives, and modifications. In particular, the steps of method of the prediction of merit and/or selection of at least one individual of interest are effected by instructions in the software that are carried out within the computer system 1000. The instructions may be formed as one or more code modules, each for performing one or more particular tasks. The software may also be divided into two separate parts, in which a first part and the corresponding code modules performs the prediction of merit and/or selection methods and a second part and the corresponding code modules manage a user interface between the first part and the user. The software may be stored in a computer readable medium, including the storage devices described below, for example. The software is loaded into the computer system 1000 from the computer readable medium, and then executed by the computer system 1000. A computer readable medium having such software or computer program recorded on it is a computer program product. The use of the computer program product in the computer system 1000 preferably effects an advantageous apparatus for prediction of merit and/or selection of at least one individual of interest.

As seen in FIG. 15, the computer system 1000 is formed by a computer module 1001, input devices such as a keyboard 1002 and a mouse pointer device 1003, and output devices including a printer 1015, a display device 1014 and loudspeakers 1017. An external Modulator-Demodulator (Modem) transceiver device 1016 may be used by the computer module 1001 for communicating to and from a communications network 1020 via a connection 1021. The network 1020 may be a wide-area network (WAN), such as the Internet or a private WAN. Where the connection 1021 is a telephone line, the modem 1016 may be a traditional “dial-up” modem. Alternatively, where the connection 1021 is a high capacity (e.g.: cable) connection, the modem 1016 may be a broadband modem. A wireless modem may also be used for wireless connection to the network 1020.

The computer module 1001 typically includes at least one processor unit 1005, and a memory unit 1006 for example formed from semiconductor random access memory (RAM) and read only memory (ROM). The module 1001 also includes an number of input/output (J/O) interfaces including an audio-video interface 1007 that couples to the video display 1014 and loudspeakers 1017, an I/O interface 1013 for the keyboard 1002 and mouse 1003 and optionally a joystick (not illustrated), and an interface 1008 for the external modem 1016 and printer 1015. In some implementations, the modem 1016 may be incorporated within the computer module 1001, for example within the interface 1008. The computer module 1001 also has a local network interface 1011 which, via a connection 1023, permits coupling of the computer system 1000 to a local computer network 1022, known as a Local Area Network (LAN). As also illustrated, the local network 1022 may also couple to the wide network 1020 via a connection 1024, which would typically include a so-called “firewall” device or similar functionality. The interface 1011 may be formed by an Ethernet™ circuit card, a wireless Bluetooth™ or an IEEE 802.21 wireless arrangement.

The interfaces 1008 and 1013 may afford both serial and parallel connectivity, the former typically being implemented according to the Universal Serial Bus (USB) standards and having corresponding USB connectors (not illustrated). Storage devices 1009 are provided and typically include a hard disk drive (HDD) 1010. Other devices such as a floppy disk drive and a magnetic tape drive (not illustrated) may also be used. An optical disk drive 1012 is typically provided to act as a non-volatile source of data. Portable memory devices, such optical disks (e.g.: CD-ROM, DVD), USB-RAM, and floppy disks for example may then be used as appropriate sources of data to the system 1000.

The components 1005 to 1013 of the computer module 1001 typically communicate via an interconnected bus 1004 and in a manner which results in a conventional mode of operation of the computer system 1000 known to those in the relevant art. Examples of computers on which the described arrangements can be practiced include IBM-PC's and compatibles, Sun Sparcstations, Apple Mac™ or alike computer systems evolved therefrom.

Typically, the application programs discussed above are resident on the hard disk drive 1010 and read and controlled in execution by the processor 1005. Intermediate storage of such programs and any data fetched from the networks 1020 and 1022 may be accomplished using the semiconductor memory 1006, possibly in concert with the hard disk drive 1010. In some instances, the application programs may be supplied to the user encoded on one or more CD-ROM and read via the corresponding drive 1012, or alternatively may be read by the user from the networks 1020 or 1022. Still further, the software can also be loaded into the computer system 1000 from other computer readable media. Computer readable media refers to any storage medium that participates in providing instructions and/or data to the computer system 1000 for execution and/or processing. Examples of such media include floppy disks, magnetic tape, CD-ROM, a hard disk drive, a ROM or integrated circuit, a magneto-optical disk, or a computer readable card such as a PCMCIA card and the like, whether or not such devices are internal or external of the computer module 1001. Examples of computer readable transmission media that may also participate in the provision of instructions and/or data include radio or infra-red transmission channels as well as a network connection to another computer or networked device, and the Internet or Intranets including e-mail transmissions and information recorded on Websites and the like.

The second part of the application programs and the corresponding code modules mentioned above may be executed to implement one or more graphical user interfaces (GUIs) to be rendered or otherwise represented upon the display 1014. Through manipulation of the keyboard 1002 and the mouse 1003, a user of the computer system 1000 and the application may manipulate the interface to provide controlling commands and/or input to the applications associated with the GUI(s).

The foregoing describes only some embodiments of the present invention, and modifications and/or changes can be made thereto without departing from the scope and spirit of the invention, the embodiments being illustrative and not restrictive

### BRIEF DESCRIPTION OF THE FIGURES

FIG. 1A is a simplified diagram showing a flow diagram of an aspect of a method for the prediction of merit of an individual;

FIG. 1B is a simplified diagram showing a flow diagram of an aspect of a method for selection of an individual based on genetic merit;

FIG. 1C is a simplified diagram showing a flow diagram of an aspect of a method for the prediction of merit and/or selection of at least one individual based on genetic merit;

FIG. 1D is a simplified diagram showing a flow diagram of an alternate aspect of a method for selection of an individual;

FIG. 1E is a simplified diagram showing a schematic outline of an arrangement of a method for obtaining a prediction for a characteristic of an individual of interest;

FIG. 1F is a simplified diagram showing a schematic outline of an arrangement of a validation technique for feature (e.g. SNP) selection and assessment;

FIG. 2 shows a graph showing molecular breeding values for kilograms of protein plotted against BLUP EBV for kilograms of protein. The MBV were weighted estimates from a genetic algorithm (GA) run modelling 500 SNP simultaneously;

FIG. 3 is a graph showing the correlation between the MBV and EBV for the bulls included in the analyses of FIG. 1, on the basis of the number of SNPs fitted in the analysis;

FIG. 4 is a graph showing the cumulative proportion of variance accounted for by the PCs when: (i) PCA is used, (ii) SPCA is used with θ=2, and (iii) SPCA is used with θ=3;

FIG. 5 is a series of exploratory plots of the BVs and the first 3 PCs for animals born before 1995 and 1995 or later. Plots above the diagonal are for the reduced data when PCA is used and plots below the diagonal are for the reduced data when SPCA is used, θ=2;

FIG. 6 is a simplified diagram showing schematic diagram for the propagation of the simulated population;

FIGS. 7(a) to 7(c) are graphs showing the mean correlation between EBV and simulated breeding value using Principal Component Analysis techniques, where there are 20 chromosomes are in the initial population, and the number of SNPs which have an additive effect is 10, 100 and 1000 respectively, and nₛₐ is the number of SNPs with an additive effect: (a) nₛₐ=10 (b) nₛₐ=100 and (c) nₛₐ=1000 SNPs over 100 iterations;

FIGS. 7(d) to 7(f) are graphs showing the mean correlation between EBV and simulated breeding value using Principal Component Analysis techniques, where there are 200 chromosomes are in the initial population, and the number of SNPs which have an additive effect is 10, 100 and 1000 respectively;

FIG. 8 is a graph showing the mean correlation between predicted breeding value and observed breeding value for real SNP data using Principal Component Analysis techniques for individuals separated into two subsets: those in the training set (K), with known EBVs, and those in the test set (U), whose EBVs are treated as unknown;

FIGS. 9A and 9B are graphs showing the correlation between predicted and true breeding values of a first generation of individuals, calculated using BLUP techniques and principal component techniques respectively;

FIGS. 1000A and 10B are graphs showing the correlation between predicted and true breeding values of the next generation of individuals, calculated using BLUP techniques and principal component techniques respectively;

FIG. 11 is a simplified diagram showing an example of the effect of prediction bias in SNP selection;

FIGS. 12A and 12B show the SNP weight distribution (i.e. VIM values) using an arrangement of the second feature selection methods;

FIGS. 13A and 13B show examples of the results from the SNP selection process;

FIGS. 14A to 14D show comparative examples of the correlation between MBV and EBV for the PLS and SVM methods of dimension reduction;

FIG. 15 shows a schematic depiction of an example apparatus for the implementation of the methods for prediction of merit and/or selection of at least one individual of interest as described herein;

FIG. 16 shows an example of the distribution plot of the number of parities per family;

FIG. 17 shows an example of a log-likelihood plots associated with a maximum likelihood estimate; and

FIG. 18 shows an example of a plot illustrating reliability of EBV from animals models.

### DETAILED DESCRIPTION

### Definitions

In the claims of this application and in the description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention. As used herein, the singular forms “a”, “an”, and “the” include the corresponding plural reference unless the context clearly dictates otherwise. Thus, for example, a reference to “a marker” includes a plurality of such markers, and a reference to “a SNP” is a reference to one or more SNPs.

It is to be clearly understood that this invention is not limited to the particular materials and methods described herein, as these may vary. It is also to be understood that the terminology used herein is for the purpose of describing particular embodiments only, and it is not intended to limit the scope of the present invention, which will be limited only by the appended claims.

Unless defined otherwise, all technical and scientific terms used herein have the same meaning as commonly understood by one of ordinary skill in the art to which this invention belongs. Although any materials and methods similar or equivalent to those described herein can be used to practise or test the present invention, the preferred materials and methods are described.

Where a range of values is expressed, it will be clearly understood that this range encompasses the upper and lower limits of the range, and all values in between these limits.

The term “ADHIS” relates to the Australian Dairy Herd Improvement Scheme.

The term “Advanced Phenotypic Value” (APV) refers to a combination of two or more phenotypic measures that are used together in an appropriate analysis to provide a prediction of the value of a specific individual for a specific end-use, such as the production of a specific component of milk.

The term “Advanced Phenotypic and Genotypic Value” (APGV) refers to a combination of the APV above with additional information such as the predicted genetic merit of the said individual for the trait in question.

The terms “animal”, “subject” and “individual” are used interchangeably to refer to an individual at any stage of life, or after death. This includes an entity prior to birth such as a fertilised ovum, either before fusion of the male and female pro-nucleus or after the pronuclei have fused to form a zygote, an embryo created by any means, including in vitro fertilization or somatic cell nuclear transfer or an individual cell of haploid (N), diploid (2N) or greater ploidy. This term also includes a cell or a cluster of cells, including stem cells and stem cell-like cells and cell lines derived therefrom, haploid gametes, and products resulting from the gametes, including embryos.

The term “allele” or “allelic” or “marker variant” refers to variation present at a defined position within a marker or specific marker sequence; in the case of a SNP this is the actual nucleotide which is present; for a SSR, it is the number of repeat sequences; for a peptide sequence, it is the actual amino acid present (see bio-marker); in the case of a marker haplotype, it is the combination of two or more individual marker variants in a specific combination (see haplotype). An “associated allele” refers to an allele at a polymorphic locus which is associated with a particular phenotype of interest, e.g. a characteristic used in assessment of livestock, a predisposition to a disorder or a particular drug response.

The term “base pair” means a pair of nitrogenous bases, each in a separate nucleotide, in which each base is present on a separate strand of DNA and the bonding of these bases joins the component DNA strands. Typically a DNA molecule contains four bases; A (adenine), G (guanine), C (cytosine), and T (thymidine).

The term “bio-marker” refers to a biological or physical characteristic at molecular, cellular or whole organism level to describe phenotype or physiological state of an individual as a diagnostic application of current state at time of measurement (e.g. in response to stress, disease, injury, environment, age, drug treatment, or other stimulus or factor), or a prognostic tool to predict future most likely performance/health status of an individual. For example, the bio-marker may be an epigenetic modification.

The term “Best Linear Unbiased Prediction” (BLUP) refers to a statistical technique which is widely used to provide prediction of genetic merit, such as estimated breeding value (EBV) The BLUP method was originally described in Henderson C. R. (1973) Sire Evaluation and Genetic Trends. in Proc. Anim. Breed. Genet. Symp. In honor of Dr. J. L. Lush. Am. Soc. Anim. Sci. and Am. Dairy Sci. Assoc. Champaign, Ill., 10-41.

The term “Breeding Value” (BV) or “Estimated Breeding Value” (EBV) refers to any prediction of the genetic merit of an individual on the basis of phenotypic observations and quantitative genetic theory.

The term “centiMorgan” (cM) refers to the genetic distance between two loci; for example the genetic distance between two loci is 1 cM if their statistically-adjusted recombination frequency is 1%; the genetic distance in cM is numerically equal to the recombination frequency (adjusted for double crossovers, interference, etc.) expressed as a percentage. Typically in mammals, a genetic distance of 1 cM can be regarded as corresponding to a physical distance of roughly one million base pairs, although this varies both between species and within the genome of an individual. However, map distance is equivalent to recombination rate only for very closely-linked loci.

The term “companion animal” refers to animals which are commonly domesticated by people and used as pets or for companionship. This includes dogs and cats, but may also include more exotic pets such as various fish, reptiles, birds, horses, rabbits, hamsters, gerbils, mice, rats and the like.

The term “epigenetic” refers to a mechanism which changes the phenotype without altering the genotype. Epigenetic changes involve mitotically heritable changes in DNA other than changes in nucleotide sequence. Genetic information provides the blueprint for the manufacture of all the proteins necessary to create a living organism, whereas epigenetic information provides additional instructions on how, where, and when the genetic information will be used. Epigenetic controls can become dysregulated in cancer cells. Such dysregulation can affect a variety of gene types, including tumour suppressor genes, oncogenes, and cancer-associated viral genes, all of which are subject to regulation by epigenetic mechanisms. A key component of epigenetic information in mammalian and other cells is DNA methylation, mostly in the promoter region. For example, tumour suppressor genes are inactivated by hypermethylation, whereas oncogenes are activated by methylation. Epigenetic markers for bladder, colon, cervical, head and neck, lung, and prostate cancer have been identified, and can be used for early detection and risk assessment of cancer. Microarray technology such as MethylScope™ (described in US patent publication No. 20040132048; available from Orion Genomics, St Louis, Mo.)) can be used to detect DNA methylation. Other epigenetic phenomena are known, including genomic imprinting in placental mammals and X-chromosome dosage compensation, post-transcriptional gene silencing (PTGS) or RNA interference and transcriptional gene silencing (TGS) seen in plants, and RNA-mediated silencing.

The term “Epistasis” is the interaction between genes at different loci, and an epistatic variation a variation arising from epistasis.

The term “information” refers to information which is indicative of, or potentially indicative of genetic differences between individuals in the population. The information is represented by the different types of data sets, such as sex, age SNPs, genotypes and haplotypes, used in the generation of the explanatory variables as defined below and a predictor function or functions. The information is generally parameters which can be measured in a population, and may vary independently, or may vary according to the sex and age of the individual.

The term “explanatory variables” refers to either products of a dimension reduction process or algorithm, for example latent components in a PLS analysis or principle components in a PCA analysis, or assigned weights or products of a genetic algorithm process.

The term “fitness” refers to an evolutionary measure, and relates to how many descendants an individual leaves in the next generations. Fitter individuals contribute more than less fit ones. Fitness in the genetic algorithm is the relative measure of the functions.

The term “genetic algorithm” refers to a class of function optimisation algorithms. Genetic algorithms are search algorithms that are based on natural selection and genetics. Generally speaking, they combine the concept of survival of the fittest with a randomized exchange of information. In each genetic algorithm generation there is a population composed of individuals. Those individuals can be seen as candidate solutions to the problem being solved. In each successive generation, a new set of individuals is created using portions of the fittest of the previous generation. However, randomized new information is also occasionally included so that important data are not lost and overlooked. A basic characteristic of a genetic algorithm is that it defines possible solutions to a problem in terms of individuals in a population.

The term “genetic merit” reflects the genetic or breeding worth of an individual with respect to its own performance, and is based on the cumulative effects of all relevant gene/genetic variants within its genome or as an assessment of the ability of the individual to transmit its genetic superiority or inferiority to its progeny/descendants.

The term “genotype” refers to the genetic constitution of an organism. This may be considered in total, or with respect to the alleles of a single gene, i.e. at a given genetic locus.

The term “haplotype” refers to a specific set or specific combination of markers at two or more markers or sites within a DNA sequence inherited together from the same individual. A haplotype may be a grouping of two or more SNPs which are physically present on the same chromosome, and which tend to be inherited together except when recombination occurs. The haplotype provides information regarding an allele of the gene, regulatory regions or other genetic sequences affecting a trait. The linkage disequilibrium and, thus, association of a SNP or a haplotype allele(s) and a trait can be strong enough to be detected using simple genetic approaches, or can require more sophisticated statistical approaches to be identified.

Some embodiments are based, in part, on a determination that SNPs, including haploid or diploid SNPs, and haplotype alleles, including haploid or diploid haplotype alleles, allow an inference to be drawn as to the trait of a subject, particularly a livestock subject. Accordingly, the methods can involve determining the nucleotide occurrence of at least 2, 3, 4, 5, 10, 20, 30, 40, 50, or more. SNPs. The SNPs can form all or part of a haplotype, wherein the method can identify a haplotype allele which is associated with the trait. Furthermore, the method can include identifying a diploid pair of haplotype alleles.

Numerous methods for identifying haplotype alleles in nucleic acid samples are known in the art. In general, nucleic acid occurrences for the individual SNPs are determined, and then combined to identify haplotype alleles. The Stephens and Donnelly algorithm (Am. J. Hum. Genet. 68: 978-989, 2001, which is incorporated herein by reference) can be applied to the data generated regarding individual nucleotide occurrences in SNP markers of the subject, in order to determine alleles for each haplotype in a subject's genotype. Other methods can be used to determine alleles for each haplotype in the subject's genotype, for example Clark's algorithm, and an EM algorithm described by Raymond and Rousset (Raymond et al. 1994. GenePop. Ver 3.0. Institut des Sciences de l'Evolution Universite de Montpellier, France. 1994).

The term “heterozygote” refers to an organism in which different alleles are found at a given locus on homologous chromosomes.

The term “homozygote” refers to an organism which has identical alleles at a given locus on homologous chromosomes.

The term “IBISS” refers to the Interactive Bovine In Silico SNP database (CSIRO Livestock Industries; www.livestockgenomics.csiro.au).

The term “infer” or “inferring”, when used in reference to a trait, means drawing a conclusion about a trait using a process of analyzing, individually or in combination, nucleotide occurrence(s) of one or more SNP(s), which can be part of one or more haplotypes, in a nucleic acid sample of the subject, and comparing the individual nucleotide occurrence(s) of the SNP(s), or combination thereof, to known relationships of nucleotide occurrence(s) of the SNP(s) and the trait. As disclosed herein, the nucleotide occurrence(s) can be identified directly by examining nucleic acid molecules, or indirectly by examining a polypeptide encoded by a particular genomic where the polymorphism is associated with an amino acid change in the encoded polypeptide.

The term “introgression” means the process of taking a gene from one population and introducing it to another, and then increasing its frequency in the new population.

The term “low dimensional space” refers to, for a database of information with many variables or unknowns, a low dimensional space refers to a subset of the information database with a reduced number of variables or unknowns, however, the low dimensional space retains substantially all the information or substantially all the relationships between the information in the information database.

The term “marker” refers to an identifiable DNA sequence which is variable (polymorphic) for different individuals within a population, and facilitates the study of inheritance of a trait or a gene. A marker at the DNA sequence level is linked to a specific chromosomal location unique to an individual's genotype and inherited in a predictable manner, and may be measured directly as a DNA sequence polymorphism, such as a single nucleotide polymorphism (SNP), restriction fragment length polymorphism (RFLP) or short tandem repeat (STR), or indirectly as a DNA sequence variant, such as a single-strand conformation polymorphism (SSCP). A marker can also be a variant at the level of a DNA-derived product, such as an RNA polymorphism/abundance, a protein polymorphism or a cell metabolite polymorphism, or any other biological characteristic which has a direct relationship with the underlying DNA variant or gene product.

The term “merit” encompasses at least (a) merit, of which genetic merit is but one type, (b) fitness for purpose; (c) susceptibility and/or predisposition to an outcome such as a disease.

The term “minimal prediction error” refers to maximising the accuracy of a prediction for example in terms of the of deviation of a true value to a predicted value.

The term “Molecular Breeding Value” (MBV) refers to an estimate of breeding value or genetic merit obtained from marker information, especially for DNA-based markers, but not restricted to DNA-based markers, for example the predicted performance derived using marker information with or without auxiliary information such as pedigree and estimated breeding values from relatives.

The term “phenotype” refers to any visible, detectable or otherwise measurable property of an organism, such as protein content of milk produced by a dairy cow, or symptoms of, or susceptibility to, a disorder.

The term “polygenic breeding value” refers to an EBV arising from a genetic evaluation in which the effects of large numbers of genes, each of which has a small effect, are analysed as a single joint effect.

The term “polymorphism” refers to the presence in a population of two or more allelic variants. Such allelic variants include sequence variation at a single base, for example a single nucleotide polymorphism (SNP). A polymorphism can be a single nucleotide difference present at a locus, or can be an insertion or deletion of one, a few or many consecutive nucleotides. It will be recognized that while the methods of the invention are exemplified primarily by the detection of SNPs, these methods or others known in the art can similarly be used to identify other types of polymorphisms, which typically involve more than one nucleotide.

The term “primer” refers to a single-stranded oligonucleotide capable of acting as a point of initiation of template-directed DNA synthesis. An “oligonucleotide” is a single-stranded nucleic acid, typically ranging in length from 2 to about 500 bases. The precise length of a primer will vary according to the particular application, but typically ranges from 15 to 30 nucleotides. A primer need not reflect the exact sequence of the template, but must be sufficiently complementary to hybridize to the template.

The term “predictor function” refers to the matrix of coefficients which have been established for each of the marker variants in the training population. The coefficients essentially represent the relationships between the marker variants (e.g. alleles) and the variation observed in the trait. To utilize the relationship, it is necessary to identify and use a marker which has a defined relationship to the coefficient.

The term “quantitative trait” refers to a phenotypic characteristic which varies in degree, and can be attributed to the interactions between two or more genes and their environment (also called polygenic inheritance).

The term “quantitative trait locus (QTL)” refers to stretches of DNA which are closely linked to the genes which underlie the quantitative trait in question. QTLs can be identified by methods such as PCR to help map regions of the genome which contain genes involved in specifying a quantitative trait. This can be an early step in identifying and sequencing these genes. A QTL affects a quantitative trait incompletely. Eye colour in humans is a qualitative trait, and the locus provides the complete effect, whereas fat yield is a quantitative trait which is affected by many loci, all of which could be considered QTL, but most of which would be too small to locate.

The term “Quantitative Trait Nucleotide” (QTN) refers to the actual variant which is responsible for the defined variation in a trait of interest.

The term “sampling” refers to choosing individual items from a larger set of items. Sampling may be random or non-random, or may be performed on the basis of a rule. The sampling may be conducted on the basis of a desired outcome, such as an improvement in a trait.

The term “single nucleotide polymorphism” (SNP) refers to common DNA sequence variations among individuals. The DNA sequence variation is typically a single base change or point mutation which results in genetic variation between individuals. The single base change can be an insertion or deletion of a base. Thus a SNP is characterized by the presence in a population of one or two, three or four nucleotides, typically less than all four nucleotides, at a particular locus in a genome.

A “trait” is a characteristic of an organism which manifests itself in a phenotype, and refers to a biological, performance or any other measurable characteristic(s), which can be any entity which can be quantified in, or from, a biological sample or organism, which can then be used either alone or in combination with one or more other quantified entities. Many traits are the result of the expression of a single gene, but some are polygenic, i.e. result from simultaneous expression of more than one gene. A “phenotype” is an outward appearance or other visible characteristic of an organism. Many different traits can be inferred by the methods disclosed herein. For any trait, a “relatively high” characteristic indicates greater than average, and a “relatively low” characteristic indicates less than average. For example “relatively high marbling” indicates more abundant marbling in meat than average marbling for a bovine population. Conversely, “relatively low marbling” indicates less abundant marbling than average marbling for a bovine population. Furthermore, in certain aspects, methods of the present invention infer that a bovine subject has a significant likelihood of having a value for a trait which is within the 5th, 10th, 20th, 25th, 30th, 40th, 50th, 60th, 70th, 75th, 80th, 90th, or 95th percentile of bovine subjects for a given trait.

“Trait performance” is a phenotypic measure, such as milk yield, or a phenotypic score in the case of type traits.

The term “tag SNP” refers to a representative single nucleotide polymorphisms (SNPs) in a region of the genome with high linkage disequilibrium.

Technical and scientific terms used herein have the meanings commonly understood by one of ordinary skill in the art to which the present invention pertains, unless otherwise defined. Reference is made herein to various methodologies known to those of skill in the art. Publications and other materials setting forth such known methodologies to which reference is made are incorporated herein by reference in their entireties as though set forth in full. Standard reference works setting forth the general principles of recombinant DNA technology include J. Sambrook et al., 1989, Molecular Cloning: A Laboratory Manual, 2d Ed., Cold Spring Harbor Laboratory Press, Cold Spring Harbor, N.Y.; P. B. Kaufman et al., (eds), 1995, Handbook of Molecular and Cellular Methods in Biology and Medicine, CRC

Press, Boca Raton; M J. McPherson (ed), 1991, Directed Mutagenesis: A Practical Approach, IRL Press, Oxford; J. Jones, 1992, Amino Acid and Peptide Synthesis, Oxford Science Publications, Oxford; B. M. Austen and O. M. R. Westwood, 1991, Protein Targeting and Secretion, IRL Press, Oxford; D. N Glover (ed), 1985, DNA Cloning, Volumes 1 and 11; M. J. Gait (ed), 1984, Oligonucleotide Synthesis; B. D. Hames and S. J. Higgins (eds), 1984, Nucleic Acid Hybridization; Quirke and Taylor (eds), 1991, PCR-A Practical Approach; Harries and Higgins (eds), 1984, Transcription and Translation; R. I. Freshney (ed), 1986, Animal Cell Culture; Immobilized Cells and Enzymes, 1986, IRL Press; Perbal, 1984, A Practical Guide to Molecular Cloning, J. H. Miller and M. P. Calos (eds), 1987, Gene Transfer Vectors for Mammalian Cells, Cold Spring Harbor Laboratory Press; M. J. Bishop (ed), 1998, Guide to Human Genome Computing, 2d Ed., Academic Press, San Diego, Calif.; L. F. Peruski and A. H. Peruski, 1997, The Internet and the New Biology. Tools for Genomic and Molecular Research, American Society for Microbiology, Washington, D.C. Standard reference works setting forth the general principles of immunology include S. Sell, 1996, Immunology, Immunopathology & Immunity, 5th Ed., Appleton & Lange, Stamford, Conn.; D. Male et al., 1996, Advanced Immunology, 3d Ed., Times Mirror Int'l Publishers Ltd., London; D. P. Stites and A. L Terr, 1991, Basic and Clinical Immunology, 7th Ed., Appleton & Lange, Norwalk, Conn.; and A. K. Abbas et al., 1991, Cellular and Molecular Immunology, W. B. Saunders Co., Philadelphia, Pa.

Any suitable materials and/or methods known to those of skill in the art can be utilized in carrying out the present invention; however, preferred materials and/or methods are described. Materials, reagents, and the like to which reference is made in the following description and examples are generally obtainable from commercial sources.

The methods of the invention identify animals which have superior traits, predicted very accurately, which can be used to identify parents of the next generation through selection. The invention provides a method for determining the optimum male and female parent to maximize the genetic components of dominance and epistasis, thus maximizing heterosis and hybrid vigour in the progeny animals.

### Livestock Animals

An objective of any genetic improvement program is to ascertain the genetic potential of individuals for a broad range of economically important traits at a very early age. While the classical breeding approach has produced steady genetic improvement in livestock species, it is limited by the fact that accurate prediction of an individual's genetic potential can only be achieved when the animal reaches adulthood (fertility and production traits), is harvested (meat quality traits), or commences training or racing (performance traits). This is particularly problematic for meat animals, since harvested animals obviously cannot enter the breeding pool. Furthermore, it is difficult to utilize the classical breeding approach for traits which are difficult or costly to measure, such as disease resistance and meat tenderness respectively.

In some aspects, the invention provides methods which use analysis of livestock genetic variation to improve the genetics of the population to produce animals with consistent desirable characteristics, such as animals which yield a high percentage of lean meat and a low percentage of fat efficiently. Thus the invention provides a method for selection and breeding of livestock subjects for a trait. The method includes inferring the genetic potential for a trait or a series of traits in a group of livestock candidates for use in breeding programs from a nucleic acid sample of the livestock candidates. The inference is made by a method which includes identifying the nucleotide occurrence of at least one SNP, wherein the nucleotide occurrence is associated with the trait or traits. Individuals are then selected from the group of candidates with a desired performance for the trait or traits for use in breeding programs. Progeny resulting from mating of selected parents would contain the optimum combination of traits, thus creating an enduring genetic pattern and line of animals with specific traits. These premium lines may be monitored for purity using the original SNP markers, which may be used to identify them from the entire population of livestock and protect them from genetic theft.

Under the current standards established by the United States Department of Agriculture (USDA), beef from bulls, steers, and heifers is classified into eight different quality grades. Beginning with the highest and continuing to the lowest, the eight quality grades are prime, choice, select, standard, commercial, utility, cutter and canner. The characteristics which are used to classify beef include age, colour, texture, firmness, and marbling, a term which is used to describe the relative amount of intramuscular fat of the beef Well-marbled beef from bulls, steers, and heifers, i.e., beef which contains substantial amounts of intramuscular fat relative to muscle, tends to be classified as prime or choice; whereas, beef which is not marbled tends to be classified as select. Beef of a higher quality grade is typically sold at higher prices than a lower grade beef For example, beef which is classified as “prime” or “choice,” typically, is sold at higher prices than beef which is classified into the lower quality grades.

Classification of beef into different quality grades occurs at the packing facility and involves visual inspection of the ribeye on a beef carcass which has been cut between the 12th and 13th rib prior to grading. However, the visual appraisal of a beef carcass cannot occur until the animal is harvested. Ultrasound can be used to give an indication of marbling prior to slaughter, but accuracy is low if ultrasound is done at a time significantly prior to harvest.

Another characteristic of beef which is desired by consumers is tenderness of the cooked product. Currently there are no procedures for identifying live animals whose beef would be tender if cooked properly. Currently there are two types of procedures which are used by researchers to assess the tenderness of meat samples after they have been aged and subsequently cooked. The first involves a subjective analysis by a panel of trained testers. The second type is characterized by methods used to cut or shear meat samples which have been removed from an animal and aged. One such method is the Wamer-Bratzler shear force procedure which involves an instrumental measurement of the force required to shear core samples of whole muscle after cooking. Neither of these procedures can be used to any practical effect in a fabrication setting as the need to age product prior to testing would lead to maintenance of inventory of fabricated product which would be cost prohibitive. Consequently, the methods are used at research facilities but not at packing plants. Accordingly, it is desirable to have new methods which can be used to identify carcasses and live cattle which have the potential to provide beef which will be tender if cooked properly.

Currently there are no cost-effective methods for identifying live cattle which give accurate prediction of the genetic potential to produce beef which is well-marbled. Such information could be used by feedlot operators to identify animals for purchase prior to finishing, to identify animals under contract for one or more premium programs administered by a packer, by feedlot managers to make management decisions regarding individual animals within a lot (including nutrition programs and sale dates), by cow-calf producers in marketing their animals to various feedlots or in making decisions regarding which animals will be sold on various carcass evaluation grids. Such information could also be used to identify cattle which are good candidates for breeding. Thus it is desirable to have a method which can be used to assess the beef marbling potential of live cattle, particularly young cattle well in advance of the arrival of the animal at the packing house.

Feedlots in the United States generally contain pens which typically have a capacity of about 200 animals, and market to packers, pens of cattle which are fed to an average endpoint. The endpoint is calculated as a number of days on feed estimated from biological type, sex, weight, and frame score. Animals are initially sorted to a pen based on the estimated number of days on feed and incoming group. However, sorting is done by a series of subjective and suboptimal parameters, as discussed herein. The cattle are fed to an endpoint in order to maximize the percentage of animals from which Grade USDA Choice beef can be obtained at slaughter without developing cattle which are too fat, and thus are discounted for insufficient red meat yield. The present invention provides a method for maximizing a physical characteristic of a bovine subject, including optimizing the percentage of bovine subjects which produce Grade USDA Choice and Prime beef in the most efficient manner.

While many visual and automated methods of measurement and selection of cattle in feedlots have been tried, such as ultrasound, none has been successful in accomplishing the desired end result, namely the ability to identify and select cattle with superior genetic potential for desirable characteristics, and then manage a given animal with known genetic potential for shipment at the optimum time, considering the animal's condition, performance and market factors, the ability to grow the animal to its optimum individual potential of physical and economic performance, and the ability to record and preserve each animal's performance history in the feedlot and carcass data from the packing plant for use in cultivating and managing current and future animals for meat production. The beef industry is extremely concerned with its decreasing market share relative to pork and poultry. However, to date it has been unable to devise a system or method to accomplish on a large scale what is needed to manage the current diversity of cattle (i.e. least about 100 different breeds and co-mingled breeds) to improve the beef product quality and uniformity fast enough to remain competitive in the race for the consumer dollar spent on meat.

Beef cattle traits which may be analyzed include, but are not limited to, marbling, tenderness, quality grade, quality yield, muscle content, fat thickness, feed efficiency, red meat yield, average daily weight gain, disease resistance, disease susceptibility, feed intake, protein content, bone content, maintenance energy requirement, mature size, amino acid profile, fatty acid profile, milk production, hide quality, susceptibility to the buller syndrome, stress susceptibility and response, temperament, digestive capacity, production of calpain, calpastatin and myostatin, pattern of fat deposition, ribeye area, fertility, ovulation rate, conception rate, fertility, heat tolerance, environmental adaptability, robustness, susceptibility to infection with and shedding of pathogens such as E. coli, Salmonella or Listeria species.

It has been difficult for the livestock industry to combine genetics for red meat yield and marbling and/or tenderness. In fact, conventional measurement techniques indicate that marbling and red meat yield tend to be antagonistic. Hence, there is a need for tools which identify superior genetic potential for the combination of red meat yield, tenderness and marbling. Another trait of interest is live cattle growth rate (average daily gain). Currently cattle producers do not have tools to identify animals with superior genetic potential for rapid growth prior to purchase. In addition, there are no methods currently available to identify animals which combine capability for superior growth rate with desirable carcass characteristics.

The invention further provides methods for selecting a given animal for shipment at the optimum time, considering the animal's genetic potential, performance and market factors, the ability to grow the animal to its optimum individual potential of physical and economic performance, and the ability to record and preserve each animal's performance history in the feedlot and carcass data from the packing plant for use in cultivating and managing current and future animals for meat production. These methods allow management of the current diversity of cattle to improve beef product quality and uniformity, thus improving revenue generated from beef sales.

The invention allows the identification of animals which have superior traits which can be used to identify parents of the next generation through selection. These methods can be imposed at the nucleus or elite breeding level where the improved traits would, through time, flow to the entire population of animals, or could be implemented at the multiplier or foundation parent level to sort parents into most genetically desirable. The optimum male and female parent can then be identified to maximize the genetic components of dominance and epistasis, thus maximizing heterosis and hybrid vigour in the market animals.

The methods and systems of the invention are particularly well suited for managing, selecting or mating bovine subjects of dairy or beef breeds. They allow for the ability to identify and monitor key characteristics of individual animals and manage those individual animals to maximize their individual potential performance and milk production or edible meat value. Therefore, the methods, systems, and compositions provided herein allow the identification and selection of cattle with superior genetic potential for desirable characteristics.

In certain embodiments, the subject is a member of a cattle breed used in beef production, such as Angus, Charolais, Limousin, Hereford, Brahman, Simmental or Gelbvieh. The methods and systems of the present invention are especially well-suited for implementation in a feedlot environment. They allow for the ability to identify and monitor key characteristics of individual animals and manage those individual animals to maximize their individual potential performance and edible meat value. Furthermore, the invention provides systems for collecting, recording and storing such data by individual animal identification so that it is usable to improve future animals bred by the producer and managed by the feedlot. The systems can utilize computer models to analyze information regarding nucleotide occurrences of SNPs and their association with traits, to predict an economic value for a bovine subject.

In certain aspects, the method further includes managing at least one of food intake, diet composition, administration of feed additives or pharmacological treatments such as vaccines, antibiotics, hormones and other metabolic modifiers, age and weight at which diet changes or pharmacological treatments are imposed, days fed specific diets, castration, feeding methods and management, imposition of internal or external measurements and environment of the bovine subject based on the inferred trait. This management results in improved, and in some examples, a maximization of physical characteristic of a bovine subject, for example to obtain a maximum amount of high grade beef from a bovine subject, and/or to increase the chances of obtaining grade USDA Choice or Prime beef, optimize tenderness, and/or maximize retail yield from the bovine subject taking into account the inputs required to reach those endpoints.

The method can be used to discriminate among those animals where interventions such as growth implants or vitamin E could provide the greatest value. For example, animals which do not have the traits to reach high choice or prime quality grades may be given growth implants until the end of the feeding period, thus maximizing feed efficiency while animals with a propensity to marble may not be implanted at the final stages of the feeding period to ensure maximum fat deposition intramuscularly.

The method also allows a feedlot and processor to predict the quality and yield grades of cattle in the system to optimize marketing of the fed animal or the product to meet target market specification. The method also provides information to the feedlot for purchase decisions based on the predicted economic returns from a specific supplier. Furthermore, the method allows the creation of integrated programs spanning breeders, producers, feedlots, packers and retailers.

Examples of feed additives used in the United States in beef production include antibiotics, flavours and metabolic modifiers. Information from SNPs could influence use of these additives and other pharmacological treatments, depending on cattle genetic potential and stage of growth relative to expected carcass composition. Examples of feeding methods include ad libitum versus restricted feeding, feeding in confined or non-confined conditions and number of feedings per day. Information from SNPs relative to cattle health, immune status or stress response could be used to influence choice of optimum feeding methods for individual cattle. These methods allow management of the current diversity of cattle to improve the beef product quality and uniformity, thus improving revenue generated from beef sales.

In another embodiment, methods are provided for selecting a given animal for shipment at the optimum time, considering the animal's condition, performance and market factors, the ability to grow the animal to its optimum individual potential of physical and economic performance, and the ability to record and preserve each animal's performance history in the feedlot and carcass data from the packing plant for use in cultivating and managing current and future animals for meat production.

Similar problems to those experienced with beef cattle and dairy cattle have been encountered with other livestock animals, such as pigs and poultry, which are intensively farmed.

In some embodiments the subject is a pig. In these embodiments, the trait can be age at puberty, reproductive potential, number of pigs farrowed alive, birth weight of pigs farrowed, longevity, weight of subject at a target time point, number of pigs weaned, percent of pigs weaned, pigs marketed/sow/year, average weaning weight of pigs, rate of gain, days to a target weight, meat quality, feed efficiency, manure characteristic, muscle content, fat content (leanness), disease resistance, disease susceptibility, feed intake, protein content, bone content, maintenance energy requirement, mature size, amino acid profile, fatty acid profile, stress susceptibility and response, digestive capacity, production of calpain, calpastatin activity and myostatin activity, pattern of fat deposition, fertility, ovulation rate, optimal diet, or conception rate. Manure characteristics include quantity, organic matter, plant nutrients, or salts.

In certain embodiments, the subject is a bird or avian species. For example, the bird or avian species can be a chicken or a turkey. In these embodiments, the trait can be egg production, feed efficiency, livability, meat yield, longevity, white meat yield, dark meat yield, disease resistance, disease susceptibility, optimal diet time to maturity, time to a target weight, weight at a target timepoint, average daily weight gain, meat quality, muscle content, fat content, feed intake, protein content, bone content, maintenance energy requirement, mature size, amino acid profile, fatty acid profile, stress susceptibility and response, digestive capacity, production of calpain, calpastatin activity and myostatin activity, pattern of fat deposition, fertility, ovulation rate, or conception rate. In one embodiment, the trait is resistance to Salmonella infection, ascites, and Listeria infection.

The egg characteristic can be quality, size, shape, shelf-life, freshness, cholesterol content, colour, biotin content, calcium content, shell quality, yolk colour, lecithin content, number of yolks, yolk content, white content, vitamin content, vitamin D content, nutrient density, protein content, albumen content, protein quality, avidin content, fat content, saturated fat content, unsaturated fat content, interior egg quality, number of blood spots, air cell size, grade, a bloom characteristic, chalaza prevalence or appearance, ease of peeling, likelihood of being a restricted egg, or Salmonella content.

Methods according to the invention can be used to infer more than one trait. For example a method of the present invention can be used to infer a series of traits. As used herein, a phenotype and a trait may be used interchangeably in some instances. Accordingly, a method of the present invention can infer, for example, quality grade, muscle content, and feed efficiency. This inference can be made using one SNP or a series of SNPs. Thus, a single SNP can be used to infer multiple traits; multiple SNPs can be used to infer multiple traits; or a single SNP can be used to infer a single trait.

In another aspect, the invention provides a method for improving profits related to selling meat from a livestock subject. The method includes drawing an inference regarding a trait of the livestock subject from a nucleic acid sample of the livestock subject. The method is typically performed by a method which includes identifying a nucleotide occurrence for at least SNP, wherein the nucleotide occurrence is associated with the trait, and wherein the trait affects the value of the animal or its products. Furthermore, the method includes managing at least one of food intake, diet composition, administration of feed additives or pharmacological treatments such as vaccines, antibiotics, hormones and other metabolic modifiers, age and weight at which diet changes or pharmacological treatments are imposed, days fed specific diets, castration, feeding methods and management, imposition of internal or external measurements and environment of the livestock subject based on the inferred trait. Then at least one livestock commercial product, typically meat or milk, is obtained from the livestock subject.

Methods according to this aspect of the invention can utilize a bioeconomic model, such as a model which estimates the net value of one or more livestock subjects on the basis of one or more traits. By this method, one trait or a series of traits are inferred, for example an inference regarding several characteristics of meat which will be obtained from the subject. The inferred trait information then can be entered into a model which uses the information to estimate a value for the livestock subject, or a product from the subject, based on the traits. The model is typically a computer model. Values for the traits can be used to segregate the animals. Furthermore, various parameters which can be controlled during maintenance and growth of the subjects can be input into the model in order to affect the way the animals are raised in order to obtain maximum value for the livestock subject when it is harvested.

In certain embodiments, meat or milk can be obtained at a time point which is affected by the inferred trait and one or more of the food intake, diet composition, and management of the livestock subject. For example, where the inferred trait of a livestock subject is high feed efficiency, which can be identified in quantitative or qualitative terms, meat or milk can be obtained at a time point which is sooner than a time point for a livestock subject with low feed efficiency. As another example, livestock subjects with different feed efficiencies can be separated, and those with lower feed efficiencies can be implanted with growth promotants or fed metabolic partitioning agents in order to maximize the profitability of a single livestock subject.

In another aspect, the invention provides methods which allow effective measurement and sorting of animals individually, accurate and complete record keeping of genotypes and traits or characteristics for each animal, and production of an economic end point determination for each animal using growth performance data. Accordingly, the present invention provides a method for sorting livestock subjects. The method includes inferring a trait for both a first livestock subject and a second livestock subject from a nucleic acid sample of the first livestock subject and the second livestock subject. The inference is made by a method which includes identifying the nucleotide occurrence of at least one SNP, wherein the nucleotide occurrence is associated with the trait. The method further includes sorting the first livestock subject and the second livestock subject based on the inferred trait.

The method can further include measuring a physical characteristic of the first livestock subject and the second livestock subject, and sorting the first livestock subject and the second livestock subject based on both the inferred trait and the measured physical characteristic. The physical characteristic can be, for example, weight, breed, type or frame size, and can be measured using many methods known in the art.

In another aspect the invention provides a method for cloning a livestock subject such as a cow or bull which has a specific trait or series of traits. The method includes identifying nucleotide occurrences of at least one or at least two SNPs for the livestock subject, isolating a progenitor cell from the livestock subject, and generating a cloned livestock from the progenitor cell. The method can further include before identifying the nucleotide occurrences, identifying the trait of the livestock subject, wherein the livestock subject has a desired trait and wherein the SNPs affect the trait.

Methods of cloning livestock are known in the art, and can be used for the present invention. For example, methods of cloning pigs have been reported (See e.g., Carter D. B., et. al., “Phenotyping of transgenic cloned piglets,” Cloning Stem Cells 4: 131-45 (2002)). For methods involving beef, milk and dairy product traits, known methods for cloning cattle can be used (See e.g., Bondioli, “Commercial cloning of cattle by nuclear transfer”, In: Symposium on Cloning Mammals by Nuclear Transplantation, Seidel (ed), pp. 35-38, (1994); Willadsen, “Cloning of sheep and cow embryos,” Genome, 31: 956, (1989); Wilson et al., “Comparison of birth weight and growth characteristics of bovine calves produced by nuclear transfer (cloning), embryo transfer and natural mating”, Animal Reprod. Sci., 38: 73-83, (1995); and Barnes et al., “Embryo cloning in cattle: The use of in vitro matured oocytes”, J. Reprod. Fert., 97: 317-323, (1993)). These methods include somatic cell cloning (See e.g., Enright B. P. et al., “Reproductive characteristics of cloned heifers derived from adult somatic cells,” Biol. Reprod., 66: 291-6 (2002); Bruggerhoff K., et al., “Bovine somatic cell nuclear transfer using recipient oocytes recovered by ovum pick-up: effect of maternal lineage of oocyte donors,” Biol. Reprod., 66: 367-73 (2002); Wilmut, I., et al., “Somatic cell nuclear transfer,” Nature, 419: 583 (2002); Galli, C., et al., “Bovine embryo technologies,” Theriogenology, 59: 599 (2003); Heyman, Y., et al., “Novel approaches and hurdles to somatic cloning in cattle,” Cloning Stem Cells, 4: 47 (2002)).

In another aspect, the invention provides a livestock subject resulting from the selection and breeding aspect or the cloning aspect of the invention, discussed above.

In another aspect, the invention provides a method of tracking a product of a livestock subject. The method includes identifying nucleotide occurrences for a series of genetic markers of the livestock subject, identifying the nucleotide occurrences for the series of genetic markers for a product sample, and determining whether the nucleotide occurrences of the livestock subject are the same as the nucleotide occurrences of the product sample. In this method identical nucleotide occurrences indicate that the product sample is from the livestock subject. The tracking method provides, for example, a method for historical and epidemiological tracking the location of an animal from embryo to birth through its growth period, to harvest and finally the retail product after it has reached the consumer. The series of genetic markers can be a series of single nucleotide polymorphisms (SNPs). The method can further include comparing the results of the above determination with a determination of whether the meat is from the livestock subject made using another tracking method. In this embodiment, the present invention provides quality control information which improves the accuracy of tracking the source of meat by a single method alone.

The nucleotide occurrence data for the livestock subject can be stored in a computer readable form, such as a database. Therefore, in one example, an initial nucleotide occurrence determination can be made for the series of genetic markers for a young livestock subject and stored in a database along with information identifying the livestock subject. Then, after meat from the livestock subject is obtained, possibly months or years after the initial nucleotide occurrence determination, and before and/or after the meat is shipped to a customer such as, for example, a wholesale distributor, a sample can be obtained from the product, meat, and nucleotide occurrence information determined using methods discussed herein. The database can then be queried using a user interface as discussed herein, with the nucleotide occurrence data from the meat sample to identify the livestock subject.

The invention in another aspect provides a method for inferring a trait of a subject from a nucleic acid sample of the subject, which includes identifying, in the nucleic acid sample, at least one nucleotide occurrence of a SNP. The nucleotide occurrence is associated with the trait, thereby allowing an inference of the trait.

In another aspect, the invention provides a method for identifying a livestock genetic marker which influences a trait. The method includes analyzing genetic markers for association with the trait. The genetic marker can be a SNP or can be at least two SNPs which influence the trait. Because the method can identify at least two SNPs, and in some embodiments, many SNPs, the method can identify not only additive genetic components, but non-additive genetic components such as dominance (i.e. dominating trait of an allele of one genomic over an allele of another gene) and epistasis (i.e. interaction between genes at different loci). Furthermore, the method can uncover pleiotropic effects of SNP alleles (i.e. SNP alleles or haplotypes effects on many different traits), because many traits can be analyzed for their association with many SNPs using methods disclosed herein.

Performance Animals

In certain embodiments, the subject is a horse. Horses of various breeds are used in racing, and management and breeding of horses for this purpose are very substantial industries. In addition to thoroughbreds, which are used in horse racing in many countries, standardbreds are used in trotting and pacing races, and quarterhorses and Arab horse are also used in racing. Horse bloodstock breeders currently rely on biomechanical, geometric, and physiological criteria to evaluate young adult horses (14 months and older) for their inherited racing and breeding potential. The size and relative positions of major muscles in the fore and hind limbs are measured to estimate stride power. Slow-motion videography is utilized to evaluate the efficiency of a horse's gait. Blood pressure and ultrasound are used to determine heart size, thickness, and stroke volume.

However, because the phenotype of an adult horse depends on the interaction of its genotype and environment, an adult phenotype does not provide an accurate prediction of the horse's genetic potential. In addition, parental phenotype is a poor predictor of offspring genotype. Phenotypically superior horses often produce below average foals, demonstrating the limitations of phenotypic analysis and performance or pedigree records such as stud books or race results in predicting breeding potential. Thoroughbreds for racing are normally selected and sold as yearlings, i.e. approximately 12-16 months old. In the absence of performance records, prospective purchasers rely largely on pedigree and physical conformation to select animals which they consider to have potential for racing success. However, because at this age a horse is still growing and developing, its physical conformation may not accurately predict its adult physical capacity and its performance.

A variety of phenotypes may be measured, especially those related to traits of interest, including those related or thought to relate to performance characteristics, physical structure or disease susceptibility. These measurements may include, but are not limited to, physiological parameters such as limb length, limb angle, muscle volume, resting heart rate, time to resting heart rate after physical exertion, blood pressure, maximum oxygen uptake (VO₂ₘₐₓ), maximum carbon dioxide production (VCO₂ₘₐₓ), blood volume at rest and exercise, rebreathing measurements of lung volumes, maximum sprint speed, heart size, and health parameters such as history of joint, skin, and diseases or conditions such as cardiovascular disease, orthopedic diseases, chronic obstructive pulmonary disease, pulmonary “bleeding” during extreme exertion, muscle diseases like exertional rhabdomyolysis, immune system disorders causing sarcoid tumours, and insect bite hypersensitivity. The condition may comprise normal, apparently normal, pre-clinical disease, overt disease, progress and/or stage of disease, undiagnosed or unclassified conditions, presence of drugs, response to exercise, response to vaccines, therapies, nutritional states and response to environmental conditions. The disease may comprise inflammation or involvement of the immune system, and conditions affecting respiratory, musculoskeletal, urinary, gastrointestinal and adnexal, cardiovascular, reticuloendothelial, nervous, special senses, reproductive, and integument systems. Such conditions in the horse include laminitis, lameness, viral or bacterial disease, colic, gastritis, gastric ulcers, respiratory ailments, epistaxis, fractures, musculoskeletal damage or disorders and joint disease.

Variables chosen for phenotypic determination may have a numerical format or can be grouped into ranges to form categorical variables. For example, a continuous variable such as a horse's maximum sprint speed can be grouped into several categories, such as fastest horses, having a sprint speed of over 17.5 metres/second; fast horses, having a sprint speed of between about 16 and 17.5 metres/second, and average horses having a sprint speed of between 15 and 16 metres/second. As will be apparent to one of skill in the art of statistical analysis, the segmentation of such variables can be chosen through groups of categorical variables according to the distribution of the continuous variable.

Horses can be screened for two genetic disorders, hyperkalaemic periodic paralysis (HYPP) and severe combined immunodeficiency disease (SCID). HYPP is a genetic disorder effecting quarterhorses which results in muscle spasms and paralysis (Rudolph, J., Spier, S. et al. (1992), “Periodic paralysis in quarter horses—a sodium-channel mutation disseminated by selective breeding,” Nature Genetics 2(2): 144-147). A PCR-based genetic test is available to identify horses with the HYPP disease allele. Breeders use this information to minimize the prevalence of HYPP in their stock or to identify animals needing treatment. SCID is a genetic disease of the immune system effecting Arabian horses (Don-van't Slot, H. and J. van der Kolk (2000), “Severe-Combined-Immunodeficiency-Disease (SCID) in the Arabian horse: a review.” Tijdschrift Voor Diergeneeskunde 125(19): 577-581; Shin, E., L. Perryman, et al. (1997), “Evaluation of a test for identification of Arabian horses heterozygous for the severe combined immunodeficiency trait,” J. American Veterinary Medical Association 211(10): 1268).). Horses carrying the SCID disease allele have dysfunctional immune systems. As with HYPP, a genetic test is available to identify carriers of the defective SCID gene.

It will be appreciated that similar performance and physical parameters and criteria to those used in the evaluation and selection of horses are also applicable to other animals used in racing, such as mules, camels and dogs. While mules are sterile, the methods and systems of the invention other than those relating to breeding can be applied to these animals. Similar performance and physical parameters and criteria may also be used in prediction of human athletic performance, particularly for sports which involve running and/or endurance, including but not limited to athletics events, swimming, rowing, kayaking, football codes (Australian Rules Football, rugby, American football, soccer), baseball, basketball and ice hockey.

In one embodiment the animal is a dog. The methods of the invention can be used to predict performance for racing dogs such as greyhounds, for dogs to be used in dog shows and breed club shows, or for working dogs such as guide dogs or other dogs used for assisting disabled people, sheep dogs, police dogs, and drug or quarantine detection dogs. The methods of the invention can also be used to predict performance for other companion animals, including those to be used for show. For example, the inference can be drawn regarding a coat or conformational characteristic or a health characteristic, for example, susceptibility to hip dysplasia, arthritis, diabetes, hypertension, atherosclerosis, autoimmune disorders, kidney disease and neurological disease. The invention is also useful for assessing complex traits such as energy metabolism, aging and breed-specific traits.

Methods according to the invention may be used in companion animal management, for example management in breeding, typically include managing at least one of food intake, diet composition, administration of feed additives or pharmacological treatments such as vaccines, antibiotics, age and weight at which diet changes or pharmacological treatments are imposed, days fed specific diets, castration, feeding methods and management, imposition of internal or external measurements and environment of the companion animal subject based on the inferred trait.

Methods according to the invention may be used to improve profits related to selling a companion animal subject; to manage companion animal subjects; to sort companion animal subjects; to improve the genetics of a companion animal population by selecting and breeding of companion animal subjects; to clone a companion animal subject with a specific genetic trait, a combination of genetic traits, or a combination of SNP markers which predict a genetic trait; to track a companion animal subject or offspring; and to diagnose or determine susceptibility to a health condition of a companion animal subject.

In another aspect, the invention provides a method for identifying a companion animal genetic marker which influences a phenotype of a genetic trait. The method includes analyzing companion animal genetic markers for association with the genetic trait. Preferably, the method involves determining nucleotide occurrences of single nucleotide polymorphisms (SNPs). Preferably, nucleotide occurrences of at least two SNPs are identified which influence the genetic trait or a group of traits.

The following table gives references for sets of markers in a variety of animal species, which may be used in the methods of the invention (refer to Table 12 for examples of marker and genome data sets within a variety of families and genus' which may be directly utilised by the methods and systems disclosed herein). In most cases the reference is to sets of markers which have been used to create linkage maps for that species.
Sheep: Crawford et al. (1995) Genetics 140: 703-724.
Beef cattle: Barendse et al. (1997) Mammalian Genome 8: 21-28.
Pig: Archibald et al. (1995) Mammalian Genome 6: 157-175.
Goat: Vaiman et al. (1996) Genetics 144: 279-305.
Deer: Slate et al. (2002) Genetics 160: 1587-97.
Horse: Guérin et al. (1999) Animal Genetics 30: 341-54.
Chicken: Levin et al. (1994) Journal of Heredity 85: 79-85.
Turkey: Burt et al. (2003) Animal Genetics 34: 399-409.
Mouse: Dietrich et al. (1994) Nature Genetics 7: 220-245.
Rat: Yamada et al. (1994) Mammalian Genome 5: 63-83.
Cat: Menotti-Raymond et al. (1999) Genomics 57: 9-23.
Dog: Werner et al. (1999) Mammalian Genome 10: 814-823
Baboon: Rogers et al (2000) Genomics 67: 237-247.
Salmon: Naish and Park (2002) Animal Genetics 33: 316-318; Beacham et al. (2003, Fishery Bulletin 101: 243-259
Rainbow trout: Sakamoto et al (2000) Genetics 155: 1331-1345.
Catfish: Waldbieser et al. (2001) Genetics 158: 727-734.

Nucleotide occurrences can be determined for essentially all, or all of the SNPs of a high-density, whole genome SNP map. This approach has the advantage over traditional approaches in that since it encompasses the whole genome, it identifies potential interactions of genomic products expressed from genes located anywhere on the genome, without requiring preexisting knowledge regarding a possible interaction between the genomic products. An example of a high-density, whole genome SNP map is a map of at least about 1 SNP per 10,000 kb, at least 1 SNP per 500 kb or about 10 SNPs per 500 kb, or at least about 25 SNPs or more per 500 kb. Definitions of densities of markers may change across the genome and are determined by the degree of linkage disequilibrium within a genome region.

Thus in embodiments where SNPs which affect the same trait and which are located in different genes are identified, the method can further include analyzing expression products of genes near the identified SNPs, to determine whether the expression products interact. Thus the present invention provides methods to detect epistatic genetic interactions. Laboratory methods for determining whether genomic products interact are well known in the art.

Where the trait is overall quality, the method can infer an overall average quality grade for a product obtained from subject. Alternatively, the method can infer the best or the worst quality grade expected for a product obtained from the subject. Additionally, as indicated above, the trait can be a characteristic used to classify the product.

The methods of the present invention which infer a trait can be used instead of present methods used to determine the trait, or can be used to provide further substantiation of a classification of milk, meat or another product using present methods.

It will also be appreciated that the methods of the invention are useful in the identification of markers useful in determination of physiological parameters, diagnosis of disease, estimation of risk of multifactorial genetic disorders; and identification of pharmacogenomic markers, in both humans and non-human animals such as livestock and performance animals. Prior art methods for analysis of genome-wide associations have been used to identify markers for conditions such as Crohn's disease (see for example WO/2007/025085) and diabetes (Sladek et al, Nature doi:1038/nature05616; 2007), and markers for longevity (WO/2006/138696). However, these studies have tended to search for markers for just one condition or disease at a time, using known disease-affected kindreds.

The invention is further described in detail by way of reference only to the following examples and drawings. These are provided by way of reference only, and are not intended to be limiting. Thus the invention encompasses any and all variations which become evident from the teaching provided herein.

The methods disclosed herein have been developed primarily for use as a computational method for prediction of the genetic and phenotypic merit of individuals based on the use of molecular breeding values (MBVs), and will be described hereinafter particularly with reference to this application. However, it will be appreciated that the methods are not limited to this particular field of use.

True breeding worth or true genetic merit of an individual cannot be measured, but is usually estimated statistically as Estimated Breeding Value (EBV), which is generally based on a statistical analysis of the performance of the individual itself and of progeny or relatives of the individual, using statistically-based analytical systems such as BLUP. However, there is a need in the art for selection methods which enable accurate selection of individuals for breeding prior to the availability of data which can only be obtained once the individual, or its relatives, have entered their productive phase. For example, this may be used to enable accurate selection of young sires for progeny testing.

A variety of potential methods for such selection, for example PCA and regression using a genetic algorithm, involve the use of both DNA-based genotypic information and indirect predictors of genotype and therefore phenotype, directly based on DNA markers as a source of biomarkers. These can be used either separately or together, and with or without statistical information, to assess individuals for their genetic merit. For example biomarkers such as hormone levels can be used with together with DNA markers to predict phenotypes. In this context the nature of genetic merit can be assessed on the basis of single or multiple genetic markers, which rank the individual for breeding worth on the basis of Molecular Breeding Values (MBV). The MBV can be obtained in addition to the pedigree information and BLUP-based information discussed above.

In accordance with at least some of the methods disclosed herein, the MBV may be derived without the need for direct pedigree or relationship information, i.e. as a function of relationships between markers, genotypes and EBV.

As will be appreciated, such genetic assay-assisted selection for individual breeding may allow selections to be made without the need for generation and phenotypic testing of progeny/descendants. In particular, such tests allow selections to be made among related individuals which do not necessarily exhibit the trait in question, and which can be used in introgression strategies to select both for the trait to be introgressed and against undesirable background traits.

In this context, the present methods relate to the use of the relationship between BLUP genetic merit and MBV genetic merit to predict the underlying true genetic merit.

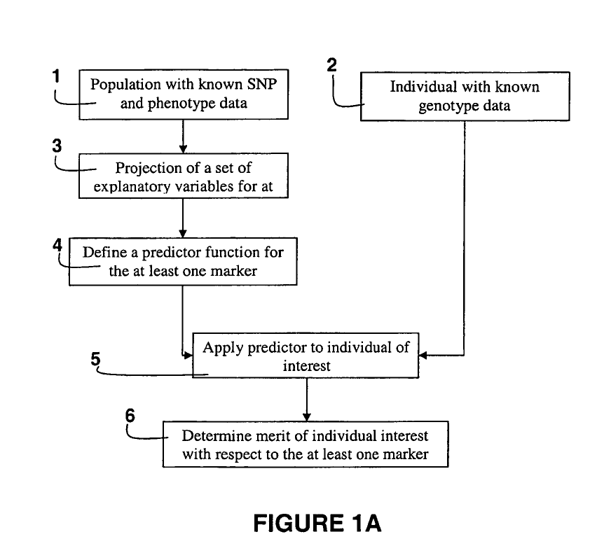

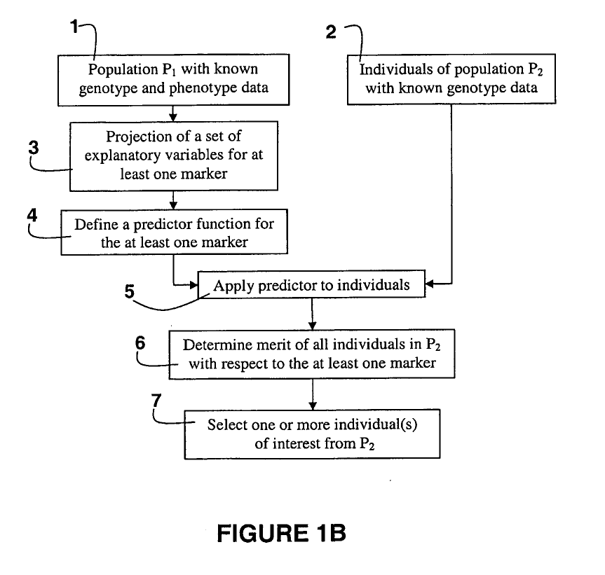

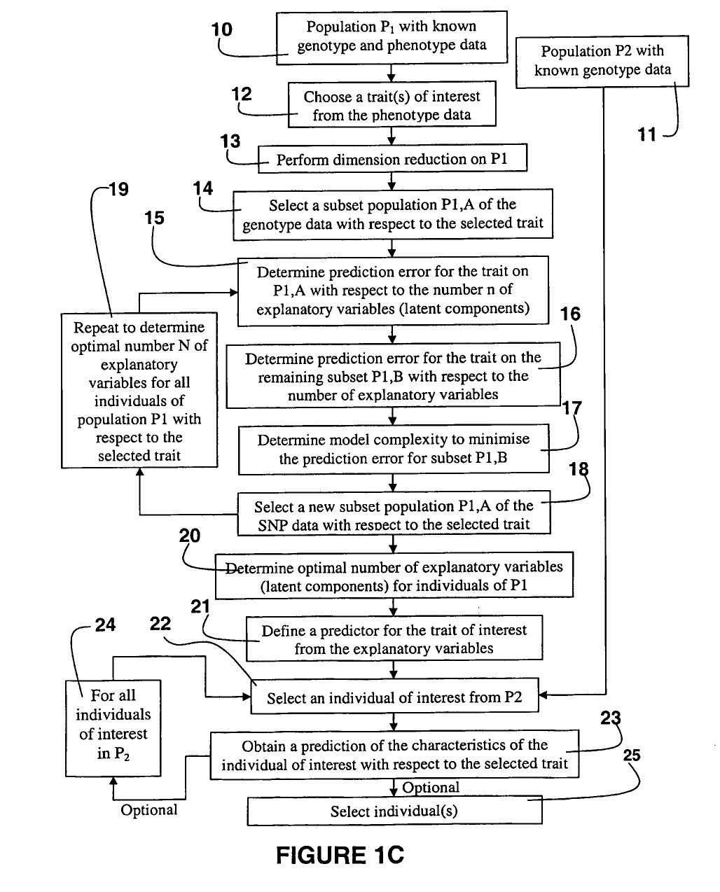

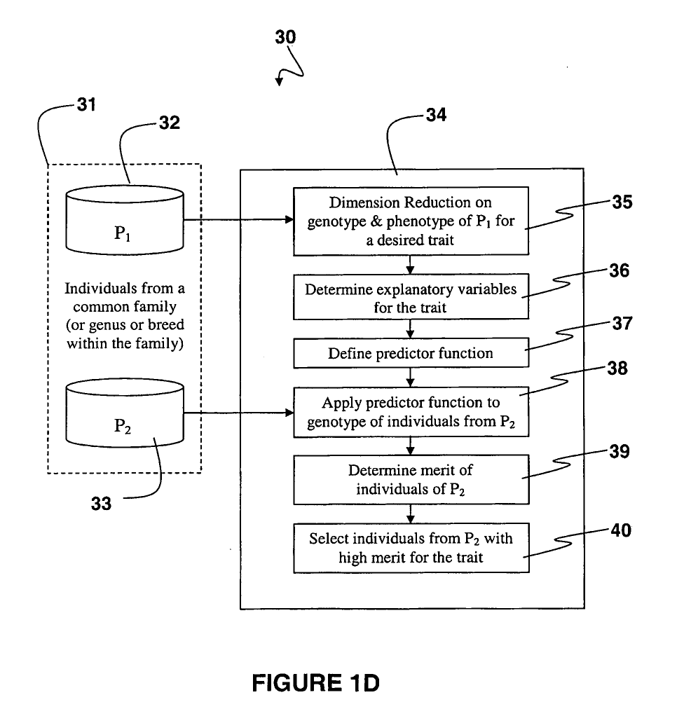

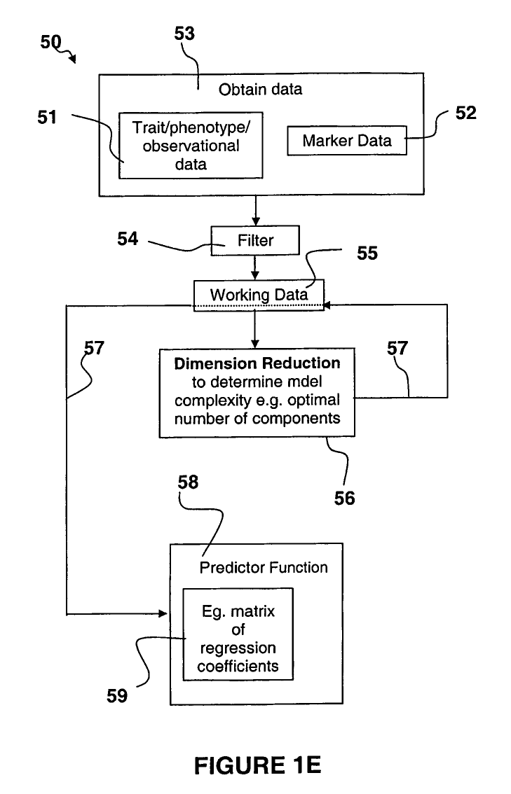

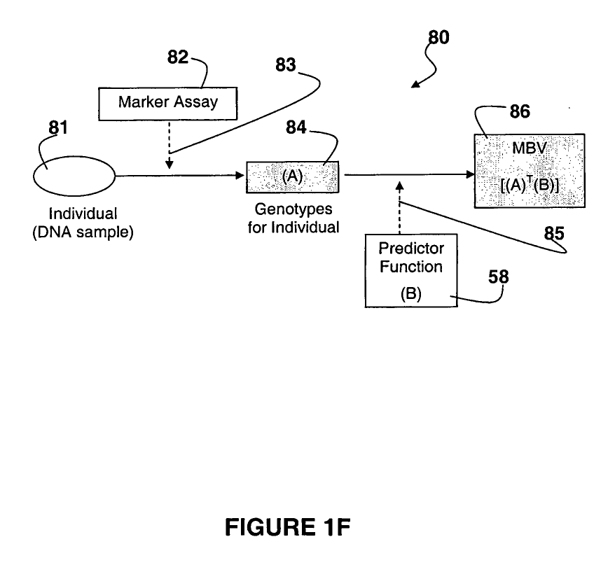

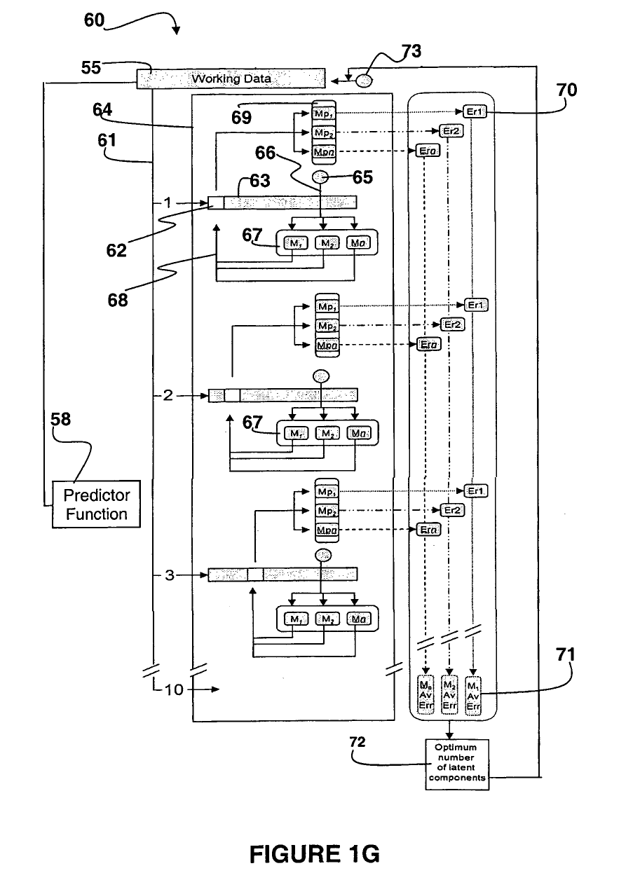

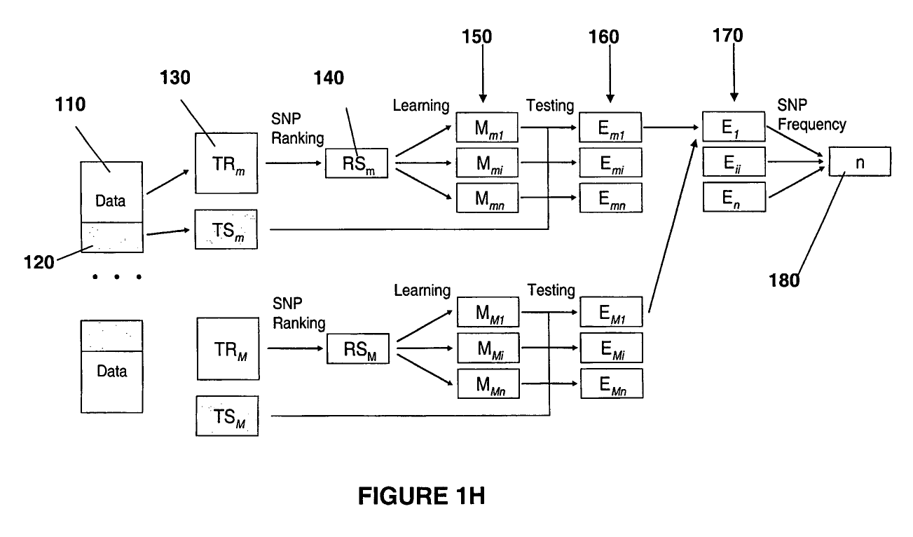

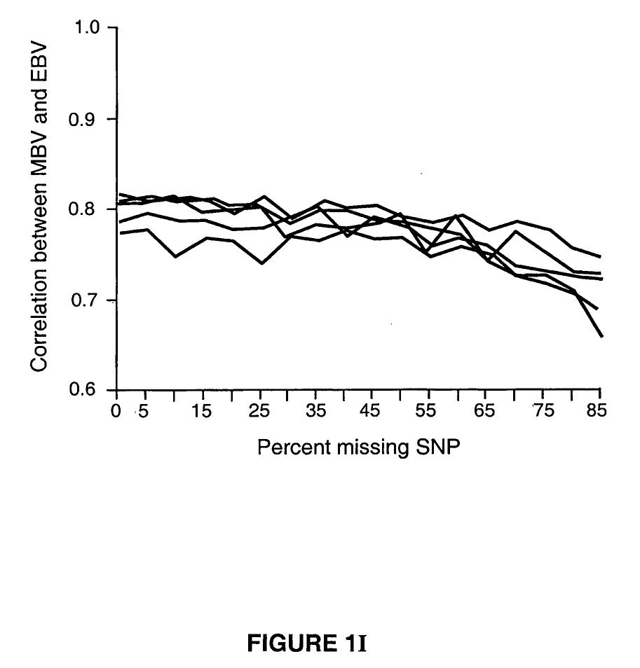

Prediction of Genetic Merit The present invention relates to methods and systems for the prediction of genetic and phenotypic merit on the basis of genome-wide marker information and example methods are exemplified in FIG. 1A to 1F. FIGS. 1A to 1F merely provide examples, which should not unduly limit the scope of the claims. One of ordinary skill in the art would recognize many variations, alternatives, and modifications Performance records of individuals and marker genotype data from which to derive prediction equations are combined with dimension reduction techniques to make predictions of merit on the basis of marker information alone, or in combination with information from other sources.

FIG. 1A shows an example arrangement of a method to predict the merit of an individual comprising the steps of: creating 1 a first population P₁, where genotypic and phenotypic information on the individuals in the first population are known; selecting an individual 2 or set of individuals forming a second population P₂, where only genotypic information on the individual(s) in P₂ are known; determining 3 a set of explanatory variables for at least one marker for individuals in the first population; defining 4 a predictor function for the at least one marker; applying 5 the predictor function to an individual of interest from P₂; and determining 6 the merit (e.g. genetic merit) of the individual of interest with respect to the marker. In an alternative arrangement, as shown in FIG. 1B, the predictor function may be applied to all individuals in the second population P₂ and determining the merit of all individuals in P₂, and then depending on the merit of each of the individuals, selecting 7 a particular individual of interest from P₂ for a purpose.

FIG. 1C shows a further arrangement of the methods disclosed herein for determining the merit and/or selecting an individual of interest from a second population having known genotype information, based upon genotype and phenotype information of individuals in a first population. Again, first and second populations are created (10 and 11 respectively) wherein the first population has known genotype and phenotype information and the second population has known genotype information only. A trait of interest is selected 12 on which a particular individual of interest from the second population will be assessed and/or selected, and a dimension reduction process as described hereunder is performed 13 on the genotype and phenotype information of individuals in the first population. As a part of the dimension reduction procedure, a subset P_{1,A} is selected 14 with respect to the selected trait and the prediction error is determined 15 for the subset P_{1,A} with respect to the number of explanatory variables used to describe the genetic date (e.g., the number of principle components for PCA or the number of latent components for PLS etc), and the prediction error is then determined for the remaining subset P_{1,b} of individuals in P₁ with respect to the number of variables, from which the model complexity is determined which minimises the prediction error for individuals in P_{1,B}. Next a new subset P_{1,A} of the first population is selected and steps 14 through 18 are repeated 19 to determine the optimal number of explanatory variables for all individuals of the first population P₁ with respect to the selected trait. Once the optimal number of explanatory variables is determined 20, a predictor (e.g. a predictor function) is defined 21 for the trait of interest from the explanatory variables. Once the predictor has been determined, then an individual of interest is selected 22 from the second population P₂ an the predictor applied 23 to the genotype data on the selected individual to obtain a prediction of the characteristics of the individual of interest with respect to the selected trait. Optionally, the steps of selection and prediction (22 and 23 respectively) may be repeated 24 for all individuals in P₂ to obtain a prediction of the characteristics of all individuals in P₂ with respect to the selected trait, from which a particular individual may be selected 25 on the basis of their predicted merit with respect to the selected trait.

FIG. 1D is a further arrangement of the prediction and selection process described herein, where for two populations P₁ and P₂ (32 and 33 respectively) selected from individuals of a common family 31 (for example any one of the bovine, ovine, porcine, avian, human or any other family as would be appreciated by the skilled addressee, or even to a particular genus of breed within the family for example the Holstien-Fresian breed of the bovine family, or human genus for individuals of a common race, geographic location etc) the following steps are taken to select a particular individual: a dimension reduction procedure such as those described herein is performed 35 on known genotypic and phenotypic information of the individuals of P₁ with respect to a selected trait and a set of explanatory variables is determined 36 with respect to that trait. A predictor function is then defines 37, and the predictor function applied 38 to known genotype information on the individuals of P₂. From the application of the predictor function, the merit of the individuals of P₂ is determined with respect to the selected trait, and one or more individuals with a high predicted merit for the selected trait may then be selected 40 for a particular purpose.

An arrangement 50 of the process of determining the predictor function of the arrangements of FIGS. 1A to 1B is exemplified in FIG. 1E wherein trait, phenotype or observational data 51 and marker data 52 is obtained 53 for a plurality of individuals of a common family/genus/breed. It will be appreciated that, due to the nature of such information, a filtering or preprocessing 54 of the data obtained in 53 may be required i.e. quality control of the data for example exclusion of DNA or SNP data according to a particular criteria which may be data duplication or low frequency (i.e. <1%) etc, (see for example Zenger et. al (2007)), and examples of such filtering are described below, although other methods of filtering the data as would be appreciated by the skilled addressee may also be employed, to obtain a working data set 55 on which the predictor function is determined. A cross-validation procedure 56 is determined to obtain the optimal model complexity of the working data for a particular reduction method (for example the optimum number of principle components for PCA or the optimal number of latent component for PLS, or other alternate methods) and the working data 55 is then analysed 57 using the optimal model complexity to obtain a predictor function 58 which may for example (i.e. depending on the chosen method) may comprise a matrix or regression components 59. In FIG. 1F an example arrangement 80 of the application of the predictor function 58 is described for a selected individual 81. In this example the predictor function is applied to predict the MBV of the selected individual 81. A marker assay 82 is obtained 83 to determine the genotype information 84 for the individual 81 and the predictor function 58 is then applied 85 to the genotype information 84, thereby to obtain a prediction of the individual's MBV 86 (or other assessment of merit of the individual as required).

FIG. 1G shows an example arrangement of the dimension reduction process 56 of FIG. 1E incorporating a PLS methodology with cross-validation 64 as described in more detail below. The working data 55 is iterated or a suitable number of times (e.g. 10). On each iteration different groups of data sets 61 are selected. Each data set 61 is divided into a randomly chosen ‘test set’ 62 (e.g. 10%) and a residual set 63 (e.g. 90%). A dimension reduction methodology 65 is applied using PLS 66 across the residual set 63 to obtain a set of 1 to n latent component models 67 (e.g. Models [M₁ to Mₙ] as described in more detail below). The prediction capability of latent component models 67 is then performance assessed 68 on the test set 62 and the performance of each Model 1 to n is recorded to obtain a plurality of Model performance variables/function Mp₁ to Mpₙ 69, from which the prediction error 70 is calculated for each of the Model performance variables/function Mp₁ to Mpₙ and each of the data sets 61. The average prediction error 71 is then calculated for each of the models with corresponding (i.e. the same) latent variables and the optimal number of latent components 72 is chosen on the basis of the minimal (i.e. the smallest) prediction error observed. A PLS regression model comprising the latent components of the minimal prediction error 72 is then fitted to the working data 55 from which the predictor function 57 is derived.

It will be appreciated by the skilled addressee that, for the arrangements as exemplified in FIGS. 1A to 1G, where the merit of an individual is determined for a particular trait and/or marker, that the process may be repeated for any number of traits and/or markers, or potentially a particular combination of at least two to any number (for example 2 to 100 or 2 to 10,000 traits/markers).

The method relates to the use of genetic markers, including genetic markers distributed across the genome in a process capable of efficiently combining marker and phenotypic information in order to produce more accurate breeding values for quantitative or qualitative traits, particularly those traits which are difficult to estimate conventionally. This process is interchangeably referred to as Genome Wide Scanning or Genome Wide Selection or by the collective abbreviation “GWS”.

The method provides a screening tool to capture as much of the additive genetic variation in production traits as possible in order to develop molecular breeding values (MBV) as a foundation for EBVs, and may also be used to capture epistatic variations in performance or to rank individuals for specific environments. This will then provide the basis to consider new advanced breeding opportunities by the creation of individuals with elite genetic profiles in combination with advanced reproductive technologies to reduce generation interval and increase selection intensity.

The method enables selection of individuals from within a population on the basis of an assessment or estimation of their merit or appropriateness for a particular end-use. The method may involve the application of a combination of a group of techniques or part thereof to the selection of individuals, e.g. animals, cells, embryos, gametes, or plants and the subsequent individuals, e.g. animals, cells, gametes, or plants, thereby selected or bred as a result, on the basis of their value or merit or fitness for purpose for a particular end-use.

Such end-uses include breeding, in which case the assessment of merit is one of genetic merit, or allocation to a desired end-use, such as the production of a specific component of milk, in which case the assessment of merit is one of a phenotypic merit with or without an assessment of genetic merit. The output may be Advanced Phenotypic and Genotypic Value (APGV).

The method may incorporate one or more of the following sources of data or information for the individuals under study or evaluation within the population, in the form of information on the individuals which may be utilised by the methods of the invention to generate a set of explanatory variables and define a predictor function. The information may include, for example, one or more of:

a) pedigree of the individual, which may include data ranging from knowledge of the sire only through to a multi-generation pedigree, where a number of maternal and/or paternal ancestors are defined; this includes pedigrees defined by reference to the inheritance by offspring of marker variants from their parents;

b) indices of genetic merit for one or more traits of interest, such as an EBV for a trait for an individual, where the EBV may be derived using statistical analysis such as BLUP, and/or derived by evaluation of progeny/descendants of the individual;

c) data on genotypes or marker variants at markers within the genome for the individual, or markers for/of the individual;

d) data on genotypes or marker variants at markers within the genome for relatives of the individual, or markers for/of the individual;

e) indices of phenotype for the individual, for relatives of the individual and for the phenotypic variation of the population, for the trait or traits of interest;

f) indices of phenotype, including bio-markers, which may in themselves be predictive of other indices of phenotype for the individual, and for relatives of the individual, and/or of underlying genetic or phenotypic variation for individuals within the population;

g) indices of epigenetic modification or status for an individual;

h) other sources of data indicative of, or potentially indicative of, genetic differences between animals.

Examples of factors which enable the process to generate useful information in a timely and cost-effective manner include:

a) access to a system to define the genotypes at a large number of markers across the whole genome or within a defined part thereof for a population of individuals;

b) access to accurate genotypic and phenotypic data for a population of individuals; the quanta of data for the individuals within the population, and the population itself, must both be of sufficient size to provide robust estimates of the genotypes or marker variant-trait relationships;

c) ready access to a database or databases wherein the data referred to above are stored;

d) a set of computational methods for the statistical analysis of data for the generation of genetic information (such as BLUP, principal component analysis, or genetic algorithms) and for the derivation of the genotypes or marker variant-trait relationships;

e) access to scientific literature and/or public databases of genomic information which enable the identification of genes which are potential candidates as contributors to variation in the trait of interest.

The above lists are respectively not exhaustive and no preference for the preferred types of information or process factors should be implied for their inclusion or placement with these lists. For example the present methods disclosed herein do not require the pedigree information for the individual to enable the prediction of merit of that individual.

Amplification of Nucleic Acids in the Analysis of Genetic Markers

Nucleic acids used as a template for amplification may be isolated from cells, tissues or other samples according to standard methodologies. For example these may find particular use in the detection of repeat length polymorphisms, such as microsatellite markers. Amplification analysis may be performed on whole cell or tissue homogenates or biological fluid samples without substantial purification of the template nucleic acid.

Pairs of primers designed to selectively hybridize to nucleic acids are contacted with the template nucleic acid under conditions that permit selective hybridization. Depending upon the desired application, high stringency hybridization conditions may be selected so as to allow hybridization only to sequences that are completely complementary to the primers. Alternatively hybridization may occur at reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles”, are conducted until a sufficient amount of amplification product is produced.

The amplified product may be detected or quantified by visual means; alternatively, the detection may involve indirect identification of the product via chemiluminescence, radioactive scintigraphy of incorporated radiolabel or fluorescent label or even via a system using electrical and/or thermal impulse signals. Typically, scoring of repeat length polymorphisms is performed on the basis of the size of the resulting amplification product.

A number of template-dependent processes may be used to amplify the oligonucleotide sequences present in a given template sample. One of the best known amplification methods is the polymerase chain reaction (PCR), which is described in detail in U.S. Pat. Nos. 4,683,195, 4,683,202 and 4,800,159, each of which is incorporated herein by reference in its entirety.

Detection of Genetic Markers for Use in the Prediction of Genetic Merit

Non-limiting examples of methods for identifying the presence or absence of a polymorphism include detection of single nucleotide polymorphisms (SNPs), haplotypes, microsatellites (simple tandem repeat STR, simple sequence repeat SSR), restriction fragment length polymorphisms (RFLP), amplified fragment length polymorphisms (AFLP), insertion-deletion polymorphism (INDEL), random amplified polymorphic DNA (RAPD), ligase chain reaction, insertion/deletions, simple sequence conformation polymorphisms (SSCP) and direct sequencing of the gene. These techniques are well known in the art; see for example Sambrook, Fritsch and Maniatis: “Molecular Cloning: A Laboratory Manual” 2^{nd} ed. Cold Spring Harbor Laboratory Press (2001).

In particular, techniques employing PCR detection are advantageous in that detection is more rapid, less labour-intensive and requires smaller sample sizes. Once an assay format has been selected, selections may be unambiguously made on the basis of genotypes assayed at any time after a nucleic acid sample can be collected from an individual, such as an infant animal, or even earlier in the case of testing of embryos in vitro, or testing of foetal offspring. Any source of DNA may be analyzed for scoring of genotype. For example, the DNA may be nuclear or mitochondrial DNA, or any other form of DNA.

The nucleic acids to be screened may be isolated from any convenient tissue, such as blood, milk, tissue, hair follicles or semen of the animal. Single cells from early-stage embryos may also be used. Peripheral blood cells are conveniently used as the source of DNA from young or adult animals. A sufficient number of cells is obtained to provide a sufficient amount of DNA for analysis, although only a minimal sample size will be needed where scoring is by amplification of nucleic acids. The DNA can be isolated from the cell sample by standard nucleic acid isolation techniques known to those skilled in the art.

Bio-Markers

In addition to genetic markers, bio-markers can also be used. The bio-marker may comprise a component which may be a RNA sequence, a peptide, including a hormone such as insulin-like growth factor-1, a steroid such as progesterone, a metabolite such as glucose, urea or an amino acid, or an immune-mediator molecule such as γ-interferon. Such molecules have potential as diagnostic aids and/or as advanced phenotypes. For example they may be used as indirect selection criteria for variation in complex traits; in many cases the bio-markers can be used in combination to define the Advanced Phenotypic Value (APV).

Bio-markers offer potential as diagnostics and/or predictors of performance, health or production traits in animals such as dairy cattle. Generally such bio-markers are measured or detected in samples such as blood or milk including somatic cells or from other easily-accessible tissues or sources, including urine, tissue biopsies, placenta post-birth, etc.

Genetic Marker Screening Platform

A number of genetic marker screening platforms are now commercially available, and can be used to obtain the genetic marker data required for the process of the present methods. In many instances, these can take the form of genetic marker testing arrays (microarrays), which allow the simultaneous testing of many thousands of genetic markers. For example, these arrays can test genetic markers in numbers of greater than 1,000, greater than 1,500, greater than 2,500, greater than 5,000, greater than 10,000, greater than 15,000, greater than 20,000, greater than 25,000, greater than 30,000, greater than 35,000, greater than 40,000, greater than 45,000, greater than 50,000 or greater than 100,000, greater than 250,000, greater than 500,000, greater than 1,000,000, greater than 5,000,000, greater than 10,000,000 or greater than 15,000,000. The nucleotide occurrence of at least 2 SNPs can be determined. At least 2 SNPs can form a haplotype, wherein the method identifies a haplotype allele which is associated with the trait. The method can include identifying a diploid pair of haplotype alleles for one or more haplotypes.

Examples of such a commercially available product for bovine genomes are those marketed by Affymetrix Inc ((http://www.affymetrix.com)) or Illumina (http://www.illumina.com). The Affymetrix Inc product was the first 10 k bovine SNP array to be commercially released. Illumina and Affymetrix also have larger SNP panels available for humans.

The 10 k SNP array has been developed from the public domain bovine sequencing consortium (http://www.affymetrix.com/products/arrays/specific/bovine.affx) using largely intronic SNPs discovered by the 6× whole genome shotgun sequencing project across 6 breeds, 1000 SNPs all coding SNPs derived from the Interactive Bovine in silico SNP database Expressed Sequence Tag (IBISS EST) comparison/alignment (CSIRO Livestock Industries: www.livestockgenomics.csiro.au). Only SNPs with a high probability of being genuine (i.e. not sequencing artifacts) have been submitted on the 10 k SNP array. The SNPs are being developed by massive multiplex padlock probe streamlining, by which 10,000 SNP genotypes can be performed in a single reaction and visualized on an Affymetrix universal genotyping array. The core elements for this system have been proven in other mammalian systems, and are available as routine services or commercially-available testing kits. Similar products for human genotyping are available, for example from Affymetrix, Illumina and Sequenom.

Statistical Analysis

Statistical and computing strategies have been developed to integrate information on individual animals and their relatives to produce estimated breeding values (EBVs) which are not biased by non-random use of sires in different regions, seasons, herds and years. The Australian Breeding Value (ABV) is a representative product from such an evaluation system for dairy cattle. Other databases in Australia include BREEDPLAN (Beef), OVIS (sheep), PIGBLUP (swine) & TREEPLAN (Forest trees).

The developments in genetic technology described above now allow large numbers of SNP genotypes to be generated for a single organism. For animal breeding, these SNPs can be used to predict the genetic merit of animals at an early stage so that a group of superior animals can be identified for further testing or breeding. The large number of SNPs that can be evaluated means that the predictor functions are contained in a high dimensional space with large empty spaces between them. This is referred to as the “Curse of Dimensionality’ (Bellman, R., 1961), which is a phenomenon which can be overcome either by adding more animals to the experiment or by reducing the dimension of the predictor space. In many cases it may not be practicable to increase the number of animals in many cases because the required increase is of order 3nₛ, where nₛ is the number of SNPs, which for GWS can typically be in the tens of thousands. Thus the present methods relate to a reduction in the dimension of the predictor space. This is usually used to reduce the dimensions of the variables to be predicted. The present method discloses the application of a number of statistical methods, such as PCA, PLS and SVM among others, to the explanatory variables, but it will be appreciated that the application of these particular dimension reduction techniques is not restricted to these methods alone.

Principal Component Analysis

A widely-used method of dimension reduction is Principal Component Analysis (PCA), which finds linear combinations of the data such that the variance is maximised. Principal component analysis (PCA) is a statistical protocol for extracting the main relations in data of high dimensionality. A common way of finding the Principal Components of a data set is by calculating the eigenvectors of the data correlation matrix. These vectors give the directions in which the data cloud is stretched most. The projections of the data on the eigenvectors are the Principal Components. The corresponding eigenvalues give an indication of the amount of information the respective Principal Components represent. Principal Components corresponding to large eigenvalues represent much information in the data set, and thus tell us much about the relations between the data points. Principal component analysis is described in, e.g., Jolliffe, Principal Component Analysis, Springer Verlag, 1986, ISBN 0-387-96269-7. This method has been widely exploited for the analysis of very large volumes of data.

In the process described herein, a SNP array, such as the Affymetrix SNP array, with SNP markers known to be located at strategic positions in the genome, either from prior QTL information and or genome gaps, is used as a basis for genome-wide selection and genotyping.

For the construction of an index relating any of the SNP markers to molecular breeding values (MBVs), several information reduction procedures were used. The primary method is a genetic algorithm (GA), described further herein. An alternative information reduction method based on principal component analysis (PCA) is also described. Both methods rely on analysis of a training data set, in which data on explanatory variables (e.g. SNP genotypes) and traits (e.g. EBVs) is available for each animal

The training dataset comprises a set of genotyped animals with multiple genome-wide markers and some performance measure, such as EBV or trait phenotype. The information reduction algorithms (GA and PCA) search for the optimal relationship of subsets of markers which maximises the prediction of the EBV in the training population. Once established via this “training set”, predictions can be made with respect to untested individuals, for which no EBV or trait measurement is available, but which have been genotyped either for all markers or for the appropriate subset of markers identified from the training set. In so doing, predictions for the EBV of an individual can be made with a very high degree of accuracy, which may be up to 0.9 or even greater. The accuracy depends on the nature of the marker and its degree of heritability. Accuracy is very high for simulated data, whereas experimental or field data are more complex, and tend to be less accurate. Regression coefficients for traits related to fitness tend to be of low heritability.

Partial Least Squares Analysis

Another widely used statistical methodology, Partial Least Squares (PLS), is a highly efficient statistical regression technique that is well suited for the analysis of whole genome scan data. This method searches for a set of components (also called factor, latent variables or latent components) that performs a simultaneous decomposition of the predictor and response variables with the constraint that these components explain as much as possible of the covariance between predictor and response.

PLS analysis methods are superior to alternatives such as principal components regression, which extracts factors to explain as much predictor sample variation without reference to the response variables. PLS has the advantage that is balances the two objectives, seeking for factors that explain both response and predictor variation.

The number of latent components to extract using PLS analysis depends on the data. Basing the model on more extracted factors improves the model fit to the observed data, but extracting too many components can cause over-fitting, that is, tailoring the model too much to the current data, to the detriment of future predictions. Procedures to choose the number of latent components are cross validation or bootstrapping.

Described hereunder is a cross-validation method to determine the number of latent components to be used in the regression.

In order to estimate the number of latent components, observation from the data were removed in a stepwise procedure, computing a prediction model based on the remaining samples and finally testing the calculated model by comparing the estimated value with the true value for the excluded observations. This process is then repeated by excluding a new selection of observations, until all observations have been excluded once. In the following discussion, the complete data set (learning set, L) consist of N objects. The learning set was partitioned in k segments (k=10) of length l (l=N/k). If k*l≠N, the k*l−N last segments contained only l−1 objects. The N-l objects form the construction data which is used to derive the predictive model using PLS, which then in turn was used to predict the removed l objects (the validation data).

The Mean Squared Error of Prediction (MSEP) was used as the objective function in model complexity selection. The k-fold cross-validation estimate is

$${MSEP}_{CV , θ} = \frac{1}{k} {\sum }_{1}^{k} \frac{1}{l} {‖ {y}_{l} - {X}_{1} {B}_{N - l , θ} ‖}^{2}$$

where θ is the number of latent components used the estimate and B_{N-1,θ} is an estimate of the regression coefficient using θ latent components based on the construction data y_{N-1} and X_{N-1}. The value of θ which minimizes the mean error rate then determines the number of latent components in the final model as described above.

In the processes described herein, a SNP array, such as for example the Affymetrix SNP array, with SNP markers known to be located at strategic positions in the genome—either prior QTL information and or genome gaps—is used as a basis for GWS and genotyping.

For the construction of a matrix of coefficients capable of relating any marker variants to variation in the trait information of the training population, several information reduction procedures were used. The primary one is a genetic algorithm (GA) described further herein. An alternative information reduction method is also described based on partial least squares analysis (PLS). Both methods rely on analysis of a training data set in which animals have data on explanatory variables (e.g. SNP genotypes) and traits (e.g. EBVs).

The training dataset of the present method comprises a set of genotyped animals with multiple genome wide markers and some performance measure such as EBV or trait phenotype. The information reduction algorithms search for the optimal relationship of subsets of markers which maximises the prediction of the EBV in the training population. Once established via this “training set”, forward predictions can be made with respect to untested individuals for which no EBV or trait measurement is available, but which have been genotyped either for all markers or for the appropriate subset of markers identified from the training set.

Principal Component Analysis

Principal Component Analysis (PCA) is a multivariate analysis technique in which the aim is to reduce the dimension of a dataset comprised of many correlated variables, while still accounting for a large proportion of the variance. Given a vector X of random variables, the first Principal Component (PC) is the linear function, w₁^{T}X such that var(w₁^{T}X) is maximised and w₁^{T}w₁=1. The j^{th} PC is the linear function, wj, which is orthogonal to all other PCs which maximises var(wⱼ^{T}X). The problem of finding PCs is equivalent to finding the eigenvalues, λ and eigenvectors, w, of the covariance matrix of X, Σ.

PCA can be used to identify redundancy or correlation among a set of measurements or variables for the purpose of data reduction. This powerful exploratory tool provides insightful graphical summaries with ability to include additional information. PCA can also be used to summarize large sets of data; identify structure and/or trends in the data; identify redundancy, correlation in the data; and produce insightful graphical displays of the results.

Described herein is a method of predicting genotypic merit using PCA regression methods applied to SNP data from the entire genome. A cross-validation method is used to select the optimal number of principal components (PCs) to use in the regression, and methods to decide which PCs to include in the model are utilized to improve the model. The methods have been applied to simulated and real data for evaluation.

Algorithm for Principal Component Analysis

The individuals of interest can be partitioned into those with estimated BVs (K) and those to have their BVs estimated (U). The animals in the set K form the training set from which to estimate parameters which are to be used to predict the BVs of the animals in the set U. The SNPs which do not show any variation are removed from the study. The remaining SNPs are arranged into a matrix x^{o}={xᵢⱼ^{o}}, where xᵢⱼ^{o} is the number of copies of one allele (0, 1 or 2) in the i^{th} SNP position for the j^{th} individual. PCA is performed

(i) for all individuals j∈K∪U and

(ii) only animals in the training set j∉K

separately to examine the effectiveness of the method when the SNP values for the training set are known, and when the SNP values of the training set are not available, but the rotation matrix is known.

The vector of SNP means, {xio.}, is computed, saved and subtracted from X^{o} to form the matrix of ns SNPs for na individuals, Xₙ_{ₛ}ₓₙ_{ₐ}={xᵢⱼ^{o}−xᵢ^{o}}. Principal component analysis is performed on the matrix X via the Expectation Maximisation (EM) algorithm as described by Roweis (1998), which has an advantage in high dimensional data because it does not require computation of the sample covariance matrix. The algorithm to find the first npc is: for i=1, 2, . . . , n_{pc} do

Choose a vector ⁱw=(ⁱw₁, ⁱw₂, . . . , ⁱwₙₛ)^{T} so that (ⁱw^{T})ⁱw=1

loop
(E step) Compute Y=((ⁱw)^{T}(ⁱw))⁻¹(ⁱw)^{T}X
(M step) Compute ⁱw^{new}=XY^{T}(YY^{T})⁻¹
Scale ⁱw^{new} such that (ⁱw^{new})^{T}(^{j}w^{new})=1

end loop

Subtract the projection of each point onto the principal component from X to obtain X^{new}.

end for

The i^{th} principal component is given by pcᵢ=(iw)^{T}X and all principal components (pc₁, pc₂ . . . pcₙ_{_{pc}}) are now ordered such that pc1 accounts for the most variation in X and pcₙ_{_{pc}} accounts for the least variation. The principal components and rotation matrix Wₙ_{ₛ}ₓₙ_{_{pc}}=(¹w, ²w, . . . , ⁿ^{_{pc}}w) are stored. A linear model of the form is fitted to the principal components:

T_{j∈K}=β₁pc_{j,1}+β₂pc_{j,2}+ . . . βₙ_{_{pc}}pc_{j,n}_{_{pc}}+ε, (1)

where ε˜N(0,σ²), Tⱼ∈K is the measurement of a particular trait or BV of individual j∈K, pc_{j,i} is the i^{th} principal component for the j^{th} individual and (β₁, β₂, . . . , βₙ_{_{pc}}) are the regression coefficients. This is referred to as Principle Component Regression (PCR).

To predict the genotypic value of the desired individuals, the estimated regression coefficients from Equation 1 are used:

T_{j∈U}^{Pred}=\hat{β}₁pc_{j,1}+\hat{β}₂pc_{j,2}+ . . . +\hat{β}ₙ_{_{pc}}pc_{j,n}_{_{pc}}. (2)

To examine the case where the SNP values of the training set are unavailable, but the rotation matrix is available, PCA is performed on the set K. It is anticipated that the use of animals in the set U may add noise to the PCs to be used in the PCR. In order to compare the accuracy of the PCR when PCA is performed on animals in the set K⊂U to when PCA is performed on animals in the set K, PCA is performed on the set K. The regression coefficients are estimated as before (Equation 1). The individuals whose breeding values are to be predicted are arranged into a matrix z^{o}={zᵢⱼ^{o}} where zᵢⱼ^{o} is the number of alleles of one type in the i^{th} SNP position for the j^{th} individual as before. The vector of mean SNP values from the training set, {xᵢ^{o}}, is subtracted from each row of z^{o} to form the matrix Z. The principal components are computed for these individuals by the equation:

{pc₁, pc₂ . . . pcₙ_{_{pc}}}=Z^{T}W (3)

These PCs are used to predict the genotypic merit through Equation 2.

Supervised Principal Component Analysis

Many SNPs may have no effect on genetic merit. The inclusion of such SNPs may add noise to procedures used to predict BVs. Supervised Principal Components Analysis (SPCA) is a method whereby a univariate regression is performed to measure the univariate effect of each gene on the BV. Only SNPs whose t-test on the regression coefficient exceeds a threshold, θ, are taken and PCA is performed on this subset of SNPs. This method is used for θ=2 (corresponding p-value≈0:05) and θ=3 (p-value≈0:003). The case of θ=0 is equivalent to PCA.

Choosing the Number of Principal Components

Classically, methods utilising the Eigenvalues corresponding to the rows of the rotation matrix have been used in order to choose the number of principal components to keep. This includes methods such as keeping principal components with eigenvalue greater than unity, Scree plot, Horn's procedure, regression methods, Bartlett's test and the broken-stick test (see, for example Johnson and Wichern (1988) and Sharma (1996)). However, we have found that such methods greatly underestimate the number of principal components needed to accurately predict genotypic merit, since not all of the important information in the SNP data is necessarily captured in the leading principal components. This is because the quantitative trait loci do not necessarily occur in areas of the chromosome where there is a large amount of variability and may be captured in PCs that account for a relatively small proportion of the overall variance.

Described hereunder is a cross-validation method to determine the number of principal components to be used in the regression. In order to estimate the number of principal components required, the breeding values of nuk=150 individuals are randomly dropped from the sample and saved. These individuals form the group of unknowns, U and the remaining individuals form the group of knowns, K. Principal component regression is performed, and the regression coefficients are estimated, with varying numbers of PCs being used in the regression. The genotypic values of the nuk individuals in U are estimated, and the correlation with their saved breeding values is examined. This process is repeated.

Selection of Principal Components

Although the PCs are ordered from the PC which accounts for the most information to the PC which accounts for the least variation, this does not necessarily imply that the first PC contains the most relevant information for predicting genetic value. Thus, the association of some of the PCs with the response variables, which accounts for a significant part of the variation of the original data, may be spurious and therefore make the linear model unsound for prediction.

Three methods are used to select the PCs. In the first method, PCs are ranked according to the proportion of variance accounted for by each PC. Secondly, the correlations are computed between each PC and the response variable. The PCs are ordered according to their absolute correlation with the response variable, so that the first PC fitted in the model is the most highly correlated with the response variable. Forward stepwise regression may also be used to build the model. Under forward stepwise regression, the k^{th} PC added is the PC which adds the most information, given that the previous (k−1) PCs have already been fitted.

The third method of ordering the PCs is a combination of the first two methods. The PCs which are most highly correlated with the BV may account for a very small proportion of the variation in the SNPs, making the PCR less robust. Similarly, the PCs which account for a large proportion of variance in the SNPs may not influence BV at all. The PCs are ranked according to |sᵢ|,

$${s}_{i} = \frac{{λ}_{i} ρ ( {pc}_{i} , BV )}{{\sum }_{j = 1}^{{n}_{pc}} {λ}_{j}}$$

where λi is the i^{th} Eigenvalue and ρ(pci; BV) is the correlation between the i^{th} PC and the BV.

A fourth possible approach, not set out here in detail, would be to use the GA described below to select the best subset of principal components for use. The principal components would form the explanatory variable inputs to the GA, for example instead of SNP genotypes.

Genetic Algorithm Process

We have developed a program for finding the molecular breeding value (MBV) or quantitative trait loci (QTL) using a genetic algorithm when there are very large numbers of explanatory variables (SNPs, genotypes, haplotypes) and relatively few observations.

A simple linear model was fitted. This contained an overall mean, a fixed (predetermined and parameterised) number of explanatory (genetic) effects and a residual. If the available data were less reliable, the inclusion of a polygenic effect would require the use of Restricted (or Residual) Maximum Likelihood (ReML). SNP effects were calculated by regression, and MBVs calculated for all individuals as the sum of the effects for each individual. These MBVs can later be compared with the EBVs of individuals, such as young bulls once their test results are analysed.

The model employed is a hierarchical model based on the Gauss-Markov theorem, including random effects, and is of the general form:

y=u+Σf(g)+e

where the observations (y) are the sum of the general mean (u), the sum of the genotype effects (the molecular breeding value=m) for the individual (Σf(g)) and a residual (e). In matrix form this is expressed (where bold type represents a matrix) as

y=Xβ+e

The normal equations are XTXβ=XTy, which may be solved by direct inversion if β is short enough, viz. \hat{β}=(X^{T}X)⁻¹X^{T}y, or by iterative means otherwise.

The errors are calculated from the general equation:

e=y−X^{\hat{β}}.

A genetic algorithm is used to find the optimum model. All models found will contribute to weighted averages of the SNP effects and MBVs.

Evaluation of Genetic Algorithm

The ratio of the sum of squares of the model to the sum of squares of the best model is the same as the ratio of the likelihoods, so weights (w) can be calculated as

(e*)^{T}e*/e^{T}e

where e* is the vector of residuals from the best model. The weights, the product of the weights by the effects (β) and MBVs (and possibly the sums of squares) are summed. When a new best model is found, the weights and the sums of variables (explanatory or MBVs) are reduced in value by 1/w (multiplication) and e* is replaced by e.

The end results are the weighted averages of the β effects for all explanatory variables, and the weighted MBVs. Different numbers of explanatory variables are fitted and in different ways. With SNPs it is possible to fit the genotypes (3) or simply the number (0, 1 or 2) of one allele (as a covariate). When more complex explanatory variables, such as haplotypes, are fitted they must be fitted as cross classified variables.

The analysis program is written in such a way that other models for evaluation can be easily substituted for the initial one. This may even include other random effects, such as a polygenic breeding value.

Using the Genetic Algorithm to Find an Optimal Model

In order to describe the GA in the terms commonly used by computer scientists working with GAs while avoiding confusion with the terms used by geneticists, it is necessary to define these terms at the outset. Thus a genetic algorithm chromosome (GAC) defines a model.

Each GAC derived for the genetic algorithm contains the explanatory variables in a model. This consists of the section of real chromosome, comprising either the loci or the haplotypes. With some models such as haplotypes there may be a variable number of categories per chromosomal segment; some could have 2, 3, 4 or more. Ideally, segments at low frequency may be amalgamated into a single group.

Prior to running the GA, XTX and XTy are created for all effects, allowing subsets to be retrieved during the GA rather than being re-calculated.

An initial population of GAC is generated by random selection of explanatory variables. All members of this population of GACs are evaluated as subsequently described.

In each round of the GA two parent GACs are chosen at random from the population. These are “mated” together to form an offspring GAC, selecting sections from each parent GAC and ensuring that the same explanatory variables do not appear twice. If they do, then others can be chosen randomly from the complete set, or from the set contained in the two parents which were not chosen. If after evaluation the offspring GAC outperforms either parent GAC, the worst parent GAC is replaced in the population by the offspring GAC. The GAC performance criterion is currently eTe, but is not restricted to this, for example, if a subset of individuals only to be predicted is included the sum of their squared prediction errors could be used.

One example of use of the GA to evaluate MBVs comprises the steps of:

A. Parameter definition
1. Total number of potential explanatory variables
2. Number of explanatory variables in the models
3. Number of observations
4. Number of individuals (includes individuals without observations
5. Number of models in the GA

B. Memory allocation and initialisation
1. declare variables
2. zero variables
3. read data
4. build complete X′X matrix (half stored)
5. build complete X′y

C. Populate the initial set of models
1. Randomly choose explanatory variables
2. Evaluate (see above)
   a. Compute MBVs and residuals
   b. Compute weights
   c. Accumulate weighted sums of MBVs and effects (\hat{β}).

D. Search with the GA until improvement ceases
1. Breed (see above)
2. Evaluate (as per step C.2.)
3. Replace parents

E. Reportage
1. Report best solution
2. Report weighted averages (and standard errors) of the MBVs and effects (\hat{β}).

F. End

The algorithm may be repeated a number of times with different numbers of explanatory variables.

Evaluation of GAC

Each GAC is evaluated by first loading the addresses of represented effects into a vector. The vector is then used to extract the subset of elements of XTX and XTy from storage. Solutions for β can be obtained by direct inversion of XTX if the number of effects is sufficiently small or by iterative means otherwise. Weighted effects (β) and MBVs (m) are accumulated, and eTe is calculated.

Partial Least Squares Analysis

Described hereunder is a process for predicting genotypic merit using PLS methods applied to SNP data from the entire genome. A cross-validation method is used for internal validation of data using cross-validation to determine a model's predictive capacity and to determine the optimal model complexity. The methods have been applied to real data for evaluation.

The PLS prediction method aims to predict q continuous response variables Y₁, . . . , Yq using p continuous explanatory variables X₁, . . . , Xp. The available data sample consisting of n observations is denoted as (\dot{x}ᵢ,\dot{y}ᵢ)_{i=1, . . . , n}, where \dot{x}ᵢ∈□^{p} and \dot{y}ᵢ∈□^{q} denote the i-th observation of the predictor and response variables, respectively. The dots denote uncentered basic data. Their removal indicates the subtraction of the sample average, i.e.:

$${x}_{i} = {\dot{x}}_{i} - \frac{1}{n} {\sum }_{j = 1}^{n} {\dot{x}}_{i}$$

$${y}_{i} = {\dot{y}}_{i} - \frac{1}{n} {\sum }_{j = 1}^{n} {\dot{y}}_{i}$$

The xi=(xi₁, . . . xip)T are collected in the n×p matrix X. Similarly, Y is the n×q matrix containing the yi=(yi₁ . . . yip)T.

$$X = ( \begin{matrix}{x}_{1}^{T} \\ … \\ {x}_{n}^{T}\end{matrix} ) , Y = ( \begin{matrix}{y}_{1}^{T} \\ … \\ {y}_{n}^{T}\end{matrix} ) .$$

PLS is based on the latent basic component decomposition:

X=TP^{T}+E

Y=TQ^{T}+F (2)

where T∈□^{n×c} is a matrix giving the latent components for the n observations. P∈□^{p×c} and Q∈□^{q×c} are matrixes of coefficients and E∈□^{n×p} and F∈□^{n×q} are matrixes of random errors.

PLS constructs a matrix of latent components T as a linear transformation of X:

T=XW (3)

where W∈□^{p×c} is a matrix of weights. The columns of W and T are denoted as wi=(w₁ᵢ, . . . wpi)T and ti=(t₁ᵢ, . . . tni)T, respectively, for i=1, . . . c. For a fixed matrix W, the random variables obtained by forming the corresponding linear transformations of X₁, Xₚ are denoted as T₁, . . . , Tc:

T₁=w₁₁X₁+ . . . +wₚ₁Xₚ,

. . . = . . .

T_{c}=w_{1c}X₁+ . . . +w_{pc}Xₚ.

The latent components are then used for prediction in place of the original variables: once T is constructed. Q is obtained as the least squares solution of Equation (2):

Q^{T}=(T^{T}T)⁻¹T^{T}Y

Finally, the matrix B of regression coefficients for the model Y=XB+F is given as:

B=WQ^{T}=W(T^{T}T)⁻¹T^{T}Y.

For a new raw observation \dot{x}₀, the prediction {circumflex over (\dot{y}₀ of the response is given by

$${{y}^{\dot{\hat{}}}}_{0} = \frac{1}{n} {\sum }_{j = 1}^{n} {\dot{y}}_{j} + {B}^{T} ( {\dot{x}}_{0} - \frac{1}{n} {\sum }_{j = 1}^{n} {\dot{x}}_{j} )$$

In PLS, dimension reduction and regression are performed simultaneously, i.e. they output the matrix of regression coefficients B as well as the matrices W, T, P and Q. In the PLS literature, the columns of T are often denoted as ‘latent variables’ or ‘scores’. P and Q are denoted as ‘X-loadings’ and ‘Y-loadings’, respectively. Latent variables and scores can be used for diagnostic purposes and for visualization.

### Algorithm for Partial Least Squares Analysis

The individuals of interest may be partitioned into those with estimated BVs (L) and those to have their BVs estimated (K). The animals in the set L form the training set from which parameters are estimated that are to be used to predict the BVs of the animals in the set K. The SNPs that do not show any variation are removed from the study. The remaining SNPs are arranged into a matrix x^{o}={xᵢⱼ^{o}}, where xᵢⱼ^{o} is the number of copies of one allele (0, 1 or 2) in the i^{th} SNP position for the j^{th} individual. PLS is performed (i) for all individuals j∈L∪K and (ii) only animals in the training set j∉L separately to examine the effectiveness of the method when the SNP values for the training set are known and when the SNP values of the training set are not available, but the rotation matrix is known.

PLS analysis was performed using a KERNEL PLS algorithm (see Dayal B. S, and J. F. Macgregor: Improved PLS Algorithms, Journal Of Chemometrics, vol. 11, 73.85 (1997)). This method is particularly efficient when the number of SNP markers is much larger than the number of responses, as it does not require the calculation of the sample covariance matrix of X. The algorithm has the following form:
1. Compute weights of the sample covariance matrix X.
2. Compute score weights.
3. Compute the loading vectors pₐ and qₐ.
4. Update the covariance matrix.
5. # store w, p, q and r in W, P, Q and R
6. Repeat steps 2 to 5 for computation of each latent vector.
7. When done computing latent vectors, the regression coefficients are given by B_{PLS}=RQ^{T}.

More rigorously, the steps of the algorithm are described as follows:

For each a=1, . . . , A, where m is the number of response variables and A are the number of PLS components to be computed:
1. If m=1
   wₐ=X^{T}Yₐ
   else
   compute qₐ, the dominant eigenvector of (Y^{T}XX^{T}Y)ₐ
   wₐ^{T}=(X^{T}Y)ₐqₐ
   wₐ=wₐ/|wₐ|
2. r₁=w₁
   rₐ=wₐ−p₁^{T}wₐr₁−p₂^{T}wₐr₂− . . . −pₐ₋₁^{T}wₐrₐ₋₁, a>1
3. tₐ=Xrₐ
   pₐ=tₐ^{T}X/tₐ^{T}tₐ
   qₐ^{T}=rₐ^{T}(X^{T}Y)ₐ/tₐ^{T}tₐ
4. (X^{T}Y)ₐ₊₁=(X^{T}Y)ₐ−pₐqₐ^{T}(tₐ^{T}tₐ)
5. W=[w₁ w₂ . . . w_{A}]
   P=[p₁ p₂ . . . p_{A}]
   Q=[q₁ q₂ . . . q_{A}]
   R=[r₁ r₂ . . . r_{A}]
6. Go to step 2 for next latent vector computation
7. Retrieve regression coefficients B_{PLS}=RQ^{T}.

### Model Validation Procedure

The critical issue in developing a “good model” is generalization. How well will the model make predictions for cases that are not in the training set? A model that is too complex may fit the noise, not just the signal, leading to overfitting

A over fit model may well describe the relationship between SNPs and EBVs of the sires used to develop the model, but may subsequently fail to provide valid predictions (molecular breeding values, MBV) in new bulls. As will be shown in the following examples, the derived PLS models show adequate fit of the data and provide valid predictions of MBV in new bulls.

Internal validation of data using cross-validation is performed to determine a model's predictive capacity and to determine the optimal model complexity (i.e. number of latent components). The number of latent components is estimated by cross-validation techniques with is the process of removing observations from the data in a stepwise procedure, computing a prediction model based on the remaining samples and finally testing the calculated model by comparing the estimated value with the true value for the excluded observations. This process is then repeated by excluding a new selection of observations, until all observations have been excluded once.

In the following discussion, the complete data set (learning set, L) consist of N objects. The learning set was partitioned in k segments (k=10) of length l(l=N/k). If k*l≠N, the k*l−N last segments contained only l−1 objects. The N−l objects form the construction data which is used to derive the predictive model using PLS, which then in turn is used to predict the removed l objects (the validation data). The mean squared error of prediction (MSEP) of Equation (1) above is used as the objective function to obtain a k-fold cross-validation estimate.

To further validate the models a different approach was applied, in which the indices of the response variable were randomly permutated so that responses do not agree with those of the SNP data. High predictive scores for randomized models indicate that the model suffers from overfitting and that fewer predictors must be used.

Feature Selection

The goal of feature selection is to identify a reduced set of non-redundant SNPs that are useful in predicting breeding values. The SNP marker set is pruned by eliminating insignificant SNP (as will be described with reference to the methods described below, in particular with reference to the VIP method). Removal of uninformative SNP decreases the noise and complexity and therefore can improve the prediction performance of the model. An issue which is tightly connected with the prediction of breeding values is gene detection, the identification of SNP whose genotypes are associated with the considered outcome. Furthermore, a reduced SNP set provides faster and more cost-effective genotyping of animals and allows to apply statistical methods (ordinary regression etc.) which can not handle the case where n<<p.

Five methods are used for feature selection. In the first, the loading vector of the first latent component of a single response PLS model, w₁ is used, where w₁ is the weight of the first latent component t₁ in the transformation matrix of Equation (3) above. This method, however, only provides limited information.

A second selection approach is based on several latent components of the PLS model and uses the weight vectors w₁, . . . , w_{c}, and has the advantage that it is capable of capturing information on a single SNP from all PLS components included in the PLS analysis. Thus it can discover non-linear patterns which the previous measure would fail to detect. The variable influence of SNP k for the a-th PLS component is defined as a function of w²ka. VIP (variable importance in projection) is the accumulated sum over all PLS dimensions of the variable influence:

$${VIP}_{Ak} = \sqrt{( {\sum }_{a = 1}^{A} ( {w}_{ak}^{2} * ( {SSY}_{a - 1} - {SSY}_{a} ) ) * \frac{K}{{SSY}_{0} - {SSY}_{A}} )}$$

where (SSYₐ₋₁−SSYₐ) is the sum of squares explained by PLS dimension a. The sum of squares of all VIP's is equal to the number of SNP (K) in the model and therefore the average VIP would be equal to 1. SNP with large VIP, larger than 1, are the most relevant for explaining Y. The VIP values reflect the importance of terms in the model both with respect to Y, i.e. its correlation to all the responses and with respect to X.

The third approach is based on finding a threshold value of w₁ and only SNP with values over the derived threshold are used for modelling. A new X-matrix is created by column-wise permutation of the elements in X. For example, this may be repeated n times, which may be 10 times or more. The new randomised X-matrix will then consist of n times the number of variables in the original X-matrix (for example, with 10715 initial SNPs and 10 iterations, the new randomized X-matrix will have 107150 variables). Using this new permuted X-matrix a new PLS model is then calculated. The SNP are then ranked according to their w₁-values. For a given rate of false positives (e.g. 1% false positives) the cutoff point will be at the 1701 (107015*0.01) largest w₁ value, for w₁ the weight of the first latent component.

After ranking the SNP according to one of the three methods above, the final predictive model is build in a serious of selection steps. At the start of the selection process, a PLS analysis is performed including only the highest ranked marker. In subsequent steps, SNP are added to the model according to their rank. A marker is retained in the final list of selected SNP if its inclusion to the model resulted in a decrease in the cross-validated prediction error.

The fourth method of feature selection is a multivariate variable selection strategy utilising a genetic algorithm (GA) search procedure (similar to that described above) coupled to the unsupervised learning algorithm of the PLS methods described above.

Genetic algorithms are variable search procedures that are based on the principle of evolution by natural selection. In the GA terminology variables are defined as genes whereas a subset of n variables that is assessed for its ability to fit a statistical model is called a chromosome. The procedure works by evolving sets of variables (GA chromosomes) that fit certain criteria from an initial random population via cycles of differential replication, recombination and mutation of the fittest chromosomes.

The GA algorithm for the present feature selection method may be implemented as follows:
1. Start with a randomly generated population of n chromosomes.
   The chromosomes have fixed length (e.g. 100 SNP markers).
2. Calculate the fitness f(x) of each chromosome x in the population.
   (e.g. f(x)=R2)
3. Repeat the following steps until n offspring have been created
   a. Select a pair of parent chromosomes from the current population, the probability of selection being an increasing function of fitness. Selection is done “with replacement,” meaning that the same chromosome can be selected more than once to become a parent.
   b. With probability pc (the “crossover probability” or “crossover rate”), cross over the pair at a randomly chosen point (chosen with uniform probability) to form two offspring. If no crossover takes place, form two offspring that are exact copies of their respective parents.
   c. Mutate the two offspring at each locus with probability pm (the mutation probability or mutation rate), and place the resulting chromosomes in the new population. If n is odd, one new population member can be discarded at random.
4. Replace the current population with the new population.
5. Repeat from step 2.

The chromosome size is fixed by an initial parameter and the GA procedure provides a large collection of chromosomes. Although these are all good solutions of the problem, it is not clear which one should be chosen for developing a final model. The fixed chromosome size implies that some of the SNP selected in the chromosome could not be contributing to the prediction accuracy of the correspondent model. For this reason there is a need to develop a single model that is, to some extent, representative of the population.

A simple strategy to follow is to use the frequency of SNP in the population of chromosomes as criteria for inclusion in a forward selection strategy. The model of choice will be the one with the highest prediction accuracy and the lower number of SNP. However alternative models with similar accuracy but larger number of SNP can also be developed. This strategy ensures that the most represented SNP in the population of chromosomes are included in a single summary model.

A fifth method for variable selection is based on uncertainty measurements (standard errors and confidence intervals) of the PLS regression coefficients. The method is based on the so-called “Jack-knife” resampling (Efron, B., & Tibshirani, R. J. (1993)) comparing perturbed model parameter estimates from cross-validation with estimates from the full model. The formula of the jack-knife estimation of the standard error for \hat{β}_{PLS} is as follows:

$${\hat{σ}}_{{β}_{PLS}}^{[ jack ]} = {[ \frac{n - 1}{n} {\sum }_{i = 1}^{n} {( {\hat{β}}_{PLS}^{( . )} - {\hat{β}}_{PLS}^{( - i )} )}^{2} ]}^{1 / 2} ,$$

where \hat{β}_{PLS}⁽⁻ⁱ⁾ is the PLS regression coefficient, the ith observation having been removed from the data set before the determination of the PLS model, and \hat{β}_{PLS}⁽⁻⁾ is the average of the n values \hat{β}_{PLS}⁽⁻ⁱ⁾.

The limits of an approximate (1−a) confidence interval for \hat{β}_{PLS} are defined as:

$${\hat{β}}_{PLS} \pm {t}_{n - 1 , α / 2} {\hat{σ}}_{{β}_{PLS}}^{[ jack ]} ,$$

where t_{n-1,a/2} is the Student (a/2)th percentile. For a chosen a, all of the variables whose PLS regression coefficients have jack-knife confidence intervals that contain zero are eliminated at the same time.

Variable selection based on the jack-knife as it is described above for the PLS regression coefficients can be applied in the same way to VIP.

The jack-knife technique is also useful for detecting outliers. Uncertainty measurements (standard errors and confidence intervals) can be computed for scores, loadings and predicted Y-values of a PLS model.

### Validation of Feature Selection

The main goal of feature selection methods described above is to select a subset of the original SNP such that the resulting model can perform well on unseen future data points. The commonly used validation strategy for the feature selection consists of:
Step 1) Selection of features by using all the data points.
Step 2) The obtained model with the selected features is validated under a validation scheme (cross-validation, bootstrapping, etc.).

In the examples below of the present case, the cross-validated prediction error is calculated within the feature-selection process. Therefore, the estimated error is optimistically biased, due to testing on samples already considered in the feature selection process.

To correct for this selection bias, cross-validation or the bootstrap validation is used external to the gene-selection process. This requires that samples in the test set must not be used in the training set.

In general the sample will be relatively small, and one would like to make full use of all available samples in SNP selection and training of the prediction rule.

The use of different training subsets results in different list of SNP, however many or most will overlap. The most frequent SNP are selected to form the final list of selected SNP.

The procedure outline is as follows:
1. Divide the data into M parts of equal size.
2. For each M-1 part DO:
   2.1. Define a series of ranked SNP d0>d1> . . . >dk using one of the selection approaches described above.
   2.2. At step i perform a forward selection starting with the current di SNP.
   2.3. Estimate the prediction error using the remaining m subset, retain the SNP if it improves the prediction error.
   2.4. Set i=i+1, repeat from step 2.2.
3. Calculate error rate at each d0˜dk level.
4. Select the top SNP with the highest frequency.

FIG. 1E shows a schematic outline of an arrangement of a validation technique for feature (e.g. SNP) selection and assessment. The data is first split into M parts of equal size. The M-1 sets 110 form the training set (TRm) and the remaining subset 120 is used as testing set (TSm) For a given training set TRₘ 130, a SNP ranking method produces a list of ranked SNP (RSm) 140. Models Mmi 150 are developed for increasing SNP subsets. The Mmi models 150 are evaluated on the TSm test data, computing the prediction error Emᵢ 160. The average error Ei 170 is obtained as

$${E}_{i} = \frac{1}{M} {\sum }_{1}^{M} {E}_{m i}$$

By then selecting the most frequent SNP, an optimal feature set n (180 of FIG. 1E) is derived.

Handling of Missing Data

Missing data is a common feature in large genomic data sets. Dealing with missing genotypes can follow different strategies. Eliminating SNP markers with incomplete observations will result in considerable information loss if many SNP have missing genotypes for various animals.

For example the percent of missing SNP genotypes was 0.8% for 16565390 data points (1546 bulls×10715 SNP). Despite this very low rate, after eliminating SNP marker with one or more missing genotypes only 68 SNP remained. In order to be able to apply dimension reduction methods to the complete SNP data we used an imputation approach, i.e. replacing each missing genotype with a predicted value. We applied imputation with the NIPALS (nonlinear iterative partial least squares) algorithm. The aim of the NIPALS algorithm is to perform principal component analysis in the presence of missing data.

A demonstration of the performance of dimension reduction by means of PLS in combination with missing SNP genotype prediction using NIPALS is shown in FIG. 1F. Missing values of SNP genotypes were randomly generated in the range of 5% up to 85% and subsequently predicted from the 1st and 2nd principal component and factor using the NIPALS algorithm. The analysis was replicated 5 times and is shown in each of the lines of FIG. 1F. For each replicate 200 animals were randomly selected as test data i.e. group of animals for which breeding value was predicted based on SNP, molecular breeding value (MBV). Animals in the test data sets did not overlap between replicates. Analyses were performed for the trait APR. The results show that even in the case of a large proportion of missing marker genotypes most of the SNPs can be reconstructed with a minimal loss of information. For example, increasing the proportion of missing genotypes from 5% to 50% results in a slight decrease of the average correlation between MBV and known breeding value (EBV) from 0.80 to 0.78.

Application to Individual Breeding Programme

The MBV estimation procedure is applicable to all traits commonly recorded by, for example, the dairy industry including individual phenotype traits such as either bull or cow fertility and semen quality etc. For example, the MBV estimation technique could be used for, but is not restricted to, phenotype traits such as APR, ASI, Protein kg, Protein Percent, Milk yield, Fat kg, Fat Percent, Overall Type, Mammary System, Stature, Udder Texture, Bone Quality, Angularity, Muzzle Width, Body Depth, Chest Width, Pin Set, Pin Sign, Foot Angle, Set Sign, Rear Leg View, Udder Depth, Fore Attachment, Rear Attachment Height, Rear Attachment Width, Centre Ligament, Teat Placement, Teat Length, Loin Strength, Milking Speed, Temperament, Like-ability, Survival, Calving Ease, Somatic Cell Count, Cow Fertility, Gestation Length, or a combination thereof.

The system described herein may be readily adapted for prediction of the ABV of an animal external to the local population of animals—such as an animal that has been imported into Australia from overseas—and the likely impact the imported animal will have on the breeding within the local population. At present, external animals—such as imported bulls in relation to the dairy industry—are usually re-ranked when used in Australia due to genotype by environment interaction (G×E), however, the addition of the environmental factors creates a large degree of uncertainty with respect to the local population. It is anticipated that the methods described herein significantly reduce the degree of uncertainty for animals which have been progeny tested overseas, which has a large impact on the generation interval and associated costs.

The methods described above will now be further described in greater detail by reference to the following specific examples, which should not be construed as in any way limiting the scope of the arrangements of the methods.

### EXAMPLES

Development of high-density large-scale single nucleotide polymorphism (SNP) genotyping platforms has opened the possibility of GWS in any species. The following examples illustrate the techniques described above when applied to a base set of dairy cattle comprising 1546 Australian progeny-tested dairy bulls which were tested for 15,036 SNP markers, leading to the following GWS platform for use in dairy cattle.

SNP Discovery

The platform is built on a commercial SNP genotyping platform (Parallele-Affymetrix) incorporating 10,410 public domain SNP markers and around 4,626 proprietary SNP markers. The proprietary markers were selected to cover regions in the genome predicted to be marker-sparse, known QTL regions, and candidate genes from the CRC-IDP candidate gene data base, using both in-silico discovery and re-sequencing strategies which included exploitation of a comparative species approach to identify candidate genes.

SNP Performance

The 22.5 million data points resulted in the following summary performance statistics;
99.4% conversion rate to genotype assays;
88.1% informative SNP markers;
91.1% placed with predicted position based on Btau3;
97.1% on an integrated bovine map,
74.6% with minor allele frequency>0.05; and
a reproducibility of 99.2% for repeat informative assayable SNPs.

After editing and correction for discordant SNPs, 10,715 high utility SNPs were used in GWS.

SNP Complexity Reduction

The challenge of dealing with over parameterized data sets where the number of SNP variables greatly exceed the number of observations is dealt with via a variety of powerful approaches for analyzing high-dimensional whole-genome SNP data such as supervised dimension reduction through partial least squares regression (PLS), and use of optimal search algorithms for exploring the parameter space were used for prediction of genetic merit based on Molecular Breeding values (MBV).

Additional non statistical SNP reduction methods will exploit use of tag SNPs in defined haplotypes. Furthermore no loss of efficiency is observed when 6000 of the available SNPs were used in GWS development.

Prediction and Validation of MBV

A remarkable feature of model selection and cross validation methods has been the accurate prediction of true breeding value (TBV) via EBV. Accuracies of prediction within the range of 0.7-0.85 in the absence of pedigree, and QTL/gene information have been obtained.

Typically only a fraction of the available SNP (<1%) are used to predict MBV for all major traits used in dairy cattle selection. Realization of GWS may therefore well represent the first true promise of DNA based technologies for livestock improvement.

Utility and Application of GWS

Deriving MBV from a population in which future predictions have to be made offers immediate use in young sire and elite dam selection. Features of GWS can be readily incorporated with advanced reproductive technologies, leading to greatly increased rates of genetic gain and potential significant cost reduction as breeding programmes move from progeny testing in sire selection to progeny validation. Use of MBV allows for screening of suitable germplasm from global sources, and may possibly extend to incorporate gene-by-environment (G×E) and gene-by-gene (G×G) and an NRM based on shared genome content in genetic evaluation. Molecular keys (coefficients) for GWS can be readily updated as new sires enter the industry.

Additional Applications

In addition to GWS, the SNP information can be used in, among other applications, the assessment of genome wide and population diversity, mate selection, management of inbreeding, study of inherited disorders, pedigree validation, assembly of the bovine Hapmap, and high-density integrated maps.

### Example 1

### Demonstration of the Genetic Algorithm

Data from two sources were analysed separately. Genotypic data were taken from either the Affymetrix 15380 SNP chip or an independent genotyping of 1282 SNPs using the Illumina platform. The Affymetrix data corresponded to 1545 bulls with EBVs in the 2006 ADHIS genetic evaluations. The Illumina data corresponded to a subset of 412 of the 1545 bulls. In relation to this, reference is made to International Patent Application No. PCT/US2006/041745 dated 25 Oct. 2006, corresponding to Australian Provisional Patent Application Nos. 2005905899 and 2005905960, the entire disclosures of each of which are incorporated herein by reference.

The SNP markers are derived from a comprehensive bank of 1545 DNA samples from all available sires which have ABVs based on progeny tests. Location knowledge was determined to choose 5000 additional markers in regions of most interest. All 1545 bulls were genotyped with the 15,000 SNP marker panel.

This provides the ability to link the discovery phase to the application phase in a single step, and to make predictions of genetic merit in young prospective bulls to be used in the Australian national dairy herd under Australian conditions. Some of the semen samples are from bulls born more than 50 years ago; thus deep pedigree structures which are essential for certain powerful statistical analyses can be structured. Of the collection of 1650 DNA samples available, some are from the sire or grandsire of a bull which has been thoroughly progeny-tested by well-accepted methods.

Editing of the Affymetrix SNP genotypes was performed to remove SNP with
(a) no genotyping data present;
(b) more than 100 unknown genotypes;
(c) a minor allele frequency of less than 0.1; and
(d) a degree of synonymy greater than 0.95.

After these edits were sequentially applied, 7420 SNP remained. The same edits were applied to the Illumina data set to leave 550 SNP. These edits may not always be applied in the future, or may be revised as necessary in accordance with requirements.

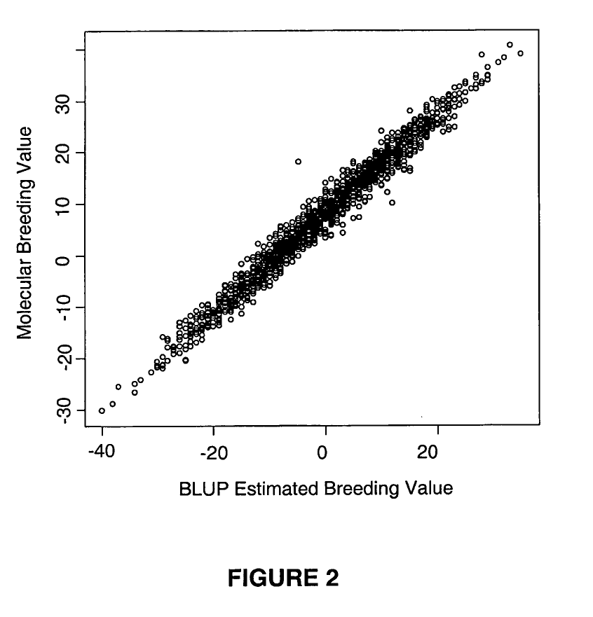

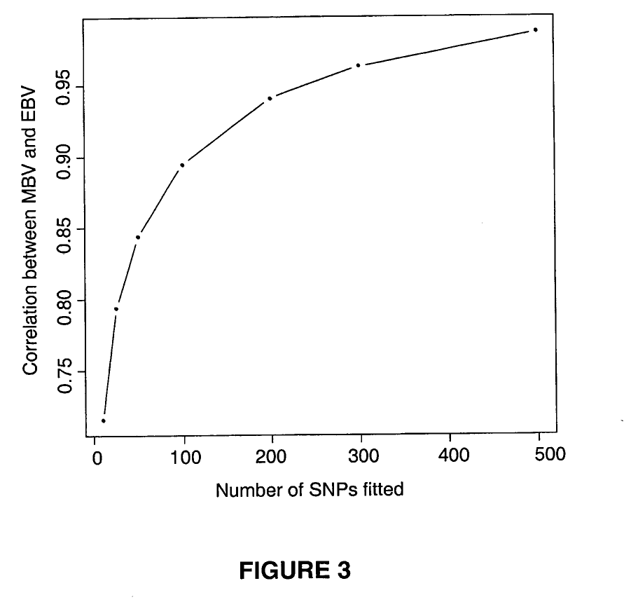

The Affymetrix data were analysed using the GA set to model 500 SNP simultaneously. The observations on the 1545 bulls used were the EBV for protein yield (kilograms of protein). The resulting estimates of MBV explained 97% of the variation in the BLUP EBVs of the 1545 bulls. FIG. 2 is a plot of MBV v EBV for this analysis. This analysis was repeated with the GA fitting either 10, 25, 50, 100, 200, 300 and 500 SNPs simultaneously. FIG. 3 shows the correlation between the MBV and EBV for the 1545 bulls included in the analyses.

Due to the limited size of the Illumina dataset, the GA was set to model 100 SNP simultaneously. Estimated breeding values for each of 38 traits and indices which showed variation for the 412 bulls were analysed. The correlations between the weighted estimates of the MBV produced and the BLUP EBV ranged from 0.83 to 0.93., as shown in Table 1.

TABLE 1
Correlations (r) between MBV and EBV of 412 bulls for each of 38
indexes and traits analysed using the Illumina genotype data and
ADHIS EBV. The GA was set to find the best 100 SNP model.

| Index or trait | r | Trait | r |
|---|---|---|---|
| APR | 0.91 | Milking Speed | 0.87 |
| ASI | 0.92 | Muzzle Width | 0.84 |
| Overall Type | 0.91 | Pin Set | 0.89 |
| Angularity | 0.89 | Pin Sign | 0.84 |
| Body Depth | 0.88 | Pin Width | 0.84 |
| Bone Quality | 0.89 | Protein % | 0.90 |
| Calving Ease | 0.93 | Protein kg | 0.93 |
| Centre Ligament | 0.91 | Rear Attachment Height | 0.92 |
| Chest Width | 0.89 | Rear Attachment Width | 0.89 |
| Cow Fertility | 0.91 | Rear Leg View | 0.90 |
| Fat | 0.87 | Set Sign | 0.83 |
| Fat % | 0.87 | Somatic Cell Count | 0.88 |
| Foot Angle | 0.89 | Stature | 0.87 |
| Fore Attachment | 0.88 | Survival | 0.90 |
| Likeability | 0.91 | Teat Length | 0.88 |
| Live Weight | 0.86 | Teat Placement | 0.89 |
| Loin Strength | 0.89 | Temperament | 0.90 |
| Mammary System | 0.92 | Udder Depth | 0.88 |
| Milk kg | 0.90 | Udder Texture | 0.93 |

### Example 1(a)

### Effectiveness of Prediction

Editing of the Affymetrix SNP genotypes was performed to remove SNP with
(a) a minor allele frequency of less than 0.1; and
(b) a degree of synonymy greater than 0.95.

After these edits were sequentially applied, 7865 SNP remained. These edits may not always be applied in the future.

The 1545 genotyped bulls were matched with a set of ADHIS evaluation results from August 2001 to give 1516 bulls with either an EBV for protein kg or a sire-maternal grandsire prediction of their 2001 EBV for protein kg. Of these 1516 bulls, 163 were born in the years 2000 or 2001, and hence would not have any progeny daughter records included in the August 2001 evaluation.

Ten random subsets of 75 bulls were selected from the 163 bull cohort and the GA run 10 times, with each of these subsets being excluded from the regression analyses but their MBV being predicted using the outcomes. Thus 1441 bulls were used in the estimation of the predictors, and 75 bulls were predicted. The GA was set to locate the best 200 SNP model. The mean correlation between MBV and EBV for the 10 groups of 75 animals was 0.74, and they ranged from 0.69 to 0.78, which is less than the 0.9+correlations between MBV and EBV for individuals in the training set.

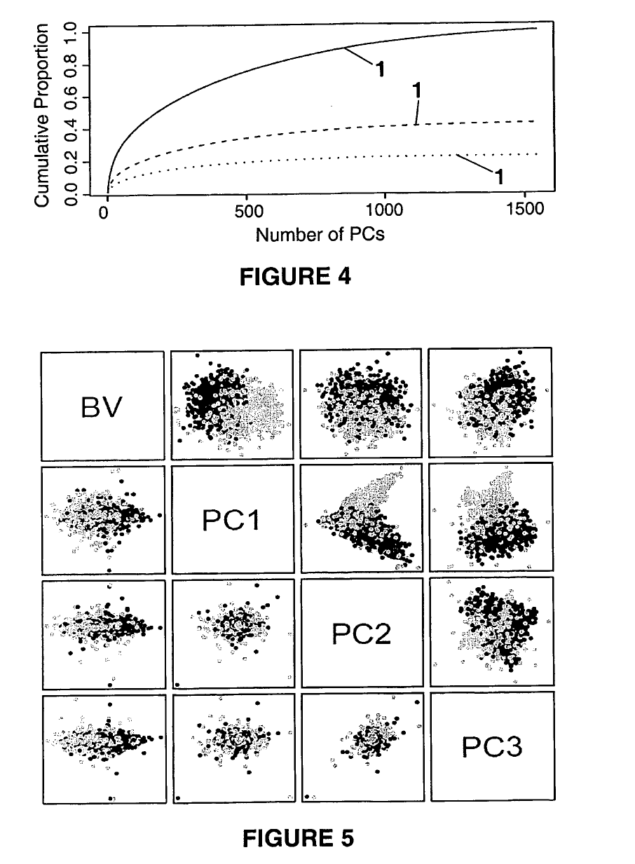

FIG. 4 displays the cumulative proportion of the variance accounted for by the PCs when PCA and SPCA are used. If all 1546 of the PCs are taken when PCA is used, clearly all of the variance of the original data is contained (line 10 of FIG. 4). The first 200 and 500 PCs account for 50% and 75% of the variation respectively when all of the SNPs are used in the reduction. The SPCA methods do not account for 100% of the total variation when all PCs are included, because not all of the original 15380 SNPs have a t-value greater than the threshold (θ). When θ=2 (line 12 of FIG. 4), 42.69% of the SNPs are taken, and these SNPs account for 35.54% of the total variation, and when θ=3 (line 14 of FIG. 4), 22.39% of the SNPs are taken, which account for 18.11% of the variation in the unedited data.

Pairwise plots of the BVs of the animals and the first 3 PCs reveal some interesting structure in the data, as displayed in FIG. 5. The plots above the diagonal are obtained when PCA is used, and plots below the diagonal are from SPCA with θ=2. FIG. 5 distinguishes between animals born before 1995 and those born in 1995 or later. This year was chosen because it divides the animals into two approximately equal groups. In the majority of plots above the diagonal in FIG. 5, the year of birth of each animal influences the distribution of points. It can be seen that animals born before 1995 tend to have lower breeding values than those born in 1995 or afterwards.

When PCA is used to reduce the data, older animals tend to have a lower score for PC1 than newer animals, indicating that PC1 is in the opposite direction to selection pressure. There are two distinct clusters in the plot of PC1 against PC2, where age defines the cluster to which animals belong. A number of outliers can also be identified from the pairwise plots which arise from PCA.

When SPCA is used to reduce the data, more outliers can be identified, and less variation is evident in the first four PCs. Animals of similar age are not grouped together when the PCs are plotted against each other, and these plots are more elliptical in shape than their counterparts which are often obtained when PCA is used.

### Example 2

### Principal Component Analysis-Simulation

Organisms having two copies of one chromosome of length 20 million base pairs were simulated. A total of 1,000 SNPs were placed on the chromosome, with their base pair positions sampled from the integers between 1 and 20 million without replacement. Some of these SNPs were simulated to have an additive effect, and these effects were sampled from a N(0,1) distribution (i.e. a Normal distribution with mean 0 and variance 1). In order to simulate the effect of Linkage Disequilibrium (LD), a small number of chromosomes, nc, was created in order to generate the base population. The number of founder chromosomes used was (i) nc=20 and (ii) nc=200. The probability of a less common allele at the i^{th} site, pi was sampled from a uniform (0,0.5) distribution (i.e. randomly sampled between 0 and 0.5), so that the matrix of haplotype values for the original chromosomes is given by:

$${B}_{ij} = \{ \begin{matrix}0 & with probability 1 - {p}_{i} \\ 1 & with probability {p}_{i}\end{matrix}$$

The top 30% of the rows of the matrix B were paired up to form males and the remaining 70% paired up to form females. Random mating was performed to produce 500 individuals. The distance between cross-overs in the breeding process was sampled from a Poisson distribution with parameter 1 million, so that each chromosome is 20 Morgans long. No mutation was simulated.

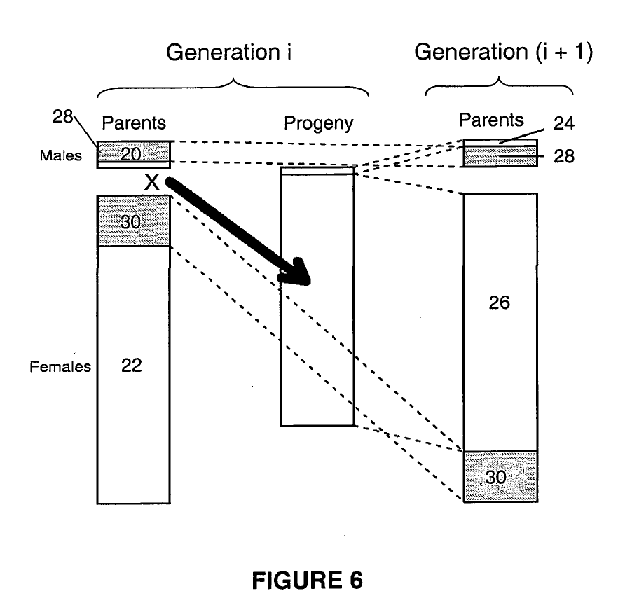

FIG. 6 is a schematic diagram of the propagation from one generation to the next. The population structure was designed to be a simplified representation of the breeding structure in place in the dairy industry in Australia. The initial population of 500 animals (generation i) was split into 40 males (20 of FIG. 6) and 460 females (22 of FIG. 6) and random breeding was simulated to form a new 395 animals 24 and 26 in the (i+1) generation in FIG. 6. Ten of these animals (24) were male and 385 (26) were female. Thirty of the males and 75 of the females from the previous generation (28 and 30 respectively) were added to the current population of 10 males and 360 females to form the next generation (not shown). This process was repeated for 10 generations, and the last three generations were stored.

The phenotypic value for each animal was calculated as:

$$T = {\sum }_{i = 1}^{i = 1000} {q}_{i} {a}_{i} + ɛ ,$$

where qᵢ is the number of less frequent alleles (0, 1 or 2) at SNP position i, aᵢ is the allelic substitution effect of the i^{th} polymorphic allele and ε is sampled from a N(0,σₑ²) distribution. The allelic substitution effect is sampled from a Gamma distribution with shape parameter 0.59 and scale parameter 7.1, with an equal probability of this effect being positive or negative. The predefined heritability (h2) and the additive genetic variance (σₐ²) determine σₑ² via the equation:

$${σ}_{e}^{2} = \frac{{σ}_{a}^{2} ( 1 - {h}^{2} )}{{h}^{2}} .$$

### Example 2(a)

### Simulation Results

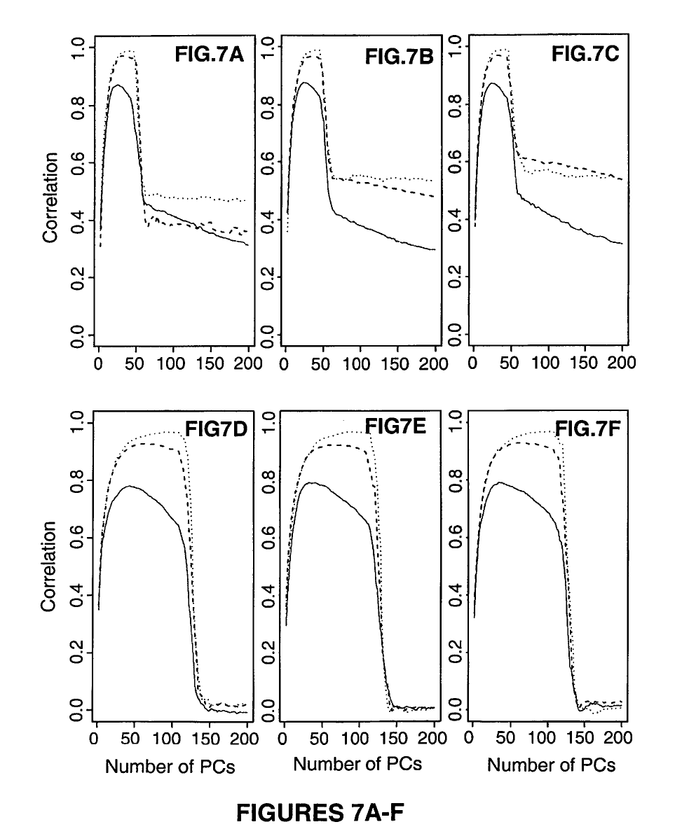

FIG. 7 examines the predictive performance of principal component regression for the simulated SNP data when h² of the trait is varied as well as the number of SNPs with an additive effect, nsa. FIGS. 7(a) to 7(f) are respectively the correlation between estimated breeding value and simulated breeding value when: (a): 10 SNPs have an additive effect and 20 chromosomes are in the initial population; (b): 100 SNPs have an additive effect and 20 chromosomes are in the initial population; (c): 1000 SNPs have an additive effect and 20 chromosomes are in the initial population; (d): 10 SNPs have an additive effect and 200 chromosomes are in the initial population; (e): 100 SNPs have an additive effect and 200 chromosomes are in the initial population; and (f): 1000 SNPs have an additive effect and 200 chromosomes are in the initial population.

The simulated heritabilities are 0.1 (-), 0.4 ( - - - ) and 0.7 ( . . . ), and each line is the mean of 50 samples. The PCs are added according to the proportion of the total variation accounted for. It can be seen that the optimal number of PCs to use is about 30 for all nine combinations of h² and nsa when nc=20 (FIGS. 7(a) to 7(c)), with correlations of greater than r=0.9 for all combinations and greater than approximately r=0.98 for heritability values of h²>0.4.

Beyond this optimal number of SNPs, spurious PCs are fitted and the correlation between the estimated and true values decreases rapidly, before this descent becomes more gentle at about 50 PCs. As expected, the heritability of the trait influences the performance of the PCR, with higher h² values allowing better prediction of genotypic merit when the optimum numbers of PCs are fitted. The influence of the number of SNPs with an effect is 22 more subtle. For low h², nsa has little effect on the performance of PCR. However, for h²=0:7, and h² 273=0:4 increasing, the number of SNPs with an additive effect from 100 to 1000 improves the performance of PCR when more than 50 PCs are fitted.

When nc=200 ((FIGS. 7(d) to 7(f)), the number of SNPs with an additive effect, nsa, has very little influence on the performance of the PCR. The h² has a larger effect when nc=200 than when nc=20, with higher h² yielding better predictive performance. More PCs are required in the regression when nc=200, with around 125 PCs needed for a h² of 0.7 for optimum predictive performance.

### Example 3

### Principal Component Analysis

### SNP Data

SNP data comprising 15380 SNPs taken from 1546 male animals born between 1955 and 2001 which come from a large recorded pedigree were used, so that breeding values were supplied for each animal along with the reliability of each estimate. Of the 23,777,480 SNP values, 7.10% are missing values. All of these missing values were replaced with is, so that all of the SNP values are consistent with Mendelian principles for the entirely male data set. If SNP data from female animals was desired to be included in the data set, any missing values could be sampled from the set of possible values given the parental genotypes. There are only males in this population, so any genotype is feasible for the sire or its offspring; if the dams' genotypes had been known, then the missing values would have been sampled from the possible set given the parents; genotypes. It will be appreciated that if the animal is the progeny of two similar homozygotes it must have the same genotype as its parents.

### Example 3(c)

### SNP Results

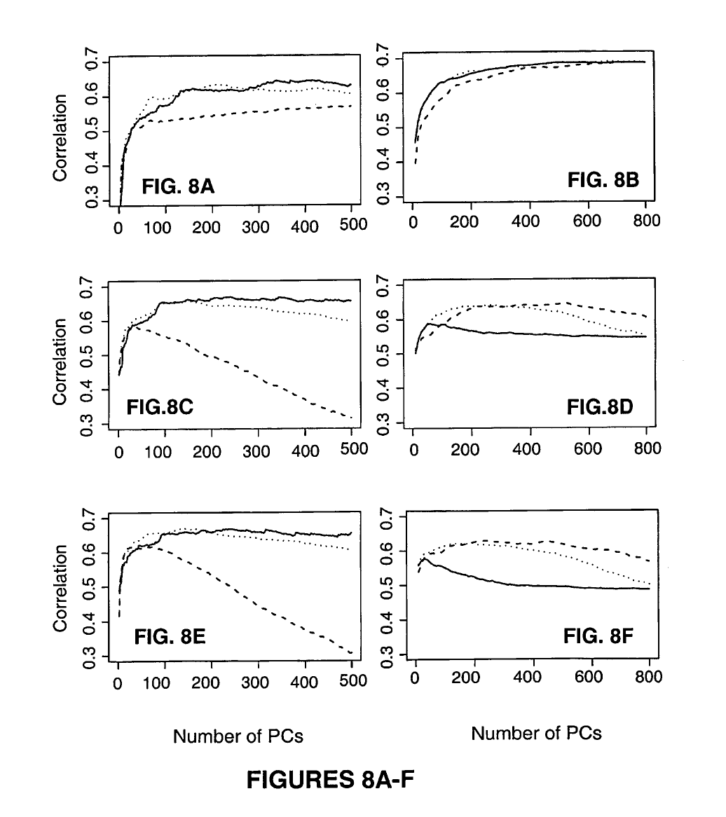

FIG. 8 shows the mean correlation between the predicted and measured genotypic merit when the cross-validation method described above is repeated 40 times (i.e. each line is the mean of 40 samples), with the PCs being added according to the proportion of variance accounted for in the unrotated data. PCs were added according to the size of the corresponding eigenvalue (-), correlation with the BVs ( - - - ) and a combination of the two methods ( . . . ). FIGS. 8(a) to 8(f) respectively refer to the cases when (a) PCA is performed on all animals (K∪U) and all SNPs, (b) PCA is performed only on animals with known BVs (K) and all SNPs, (c) PCA is performed on all animals (K∪U) and SNPs with θ>2, (d) PCA is performed only on animals with known BVs (K) and SNPs with θ>2, (e) PCA is performed on all animals (K∪U) and SNPs with θ>3, (f) PCA is performed only on animals with known BVs (K) and SNPs with θ>3.

When all SNPs are used in all animals (FIG. 8(a)), the mean correlation reaches a maximum of 0.65 when 300 to 500 PCs are fitted according to their eigenvalues, and gradually reduces as more PCs are fitted. Before this maximum is reached the curve is not monotonically increasing, with the inclusion of some PCs in the regression reducing the predictive performance of the model. When PCs are added according to the correlation with the known BVs a maximum of 0.57 is obtained, and when PCs are added according to the value of |sᵢ| the maximum is 0.63.

There is a slight improvement in predictive performance when SPCA is used on all individuals (FIGS. 8(c) and (e)). This improvement is greatest for θ=3, where a maximum mean correlation of 0.67 is obtained for methods adding PCs to the regression according to λi and according to si. When the correlation between the PCs and BVs is used to determine the order in which PCs are added, the maximum is reached after relatively few PCs, but then falls away quickly.

The best predictive model for these data is when PCA is performed on individuals with known breeding values (FIG. 8(b)). A maximum mean correlation of 0.69 is obtained for all three methods of adding PCs to the regression when more than 600 PCs are added. When SPCA is used only on the individuals with known BVs, the estimates are further from the known BVs.

### Example 4

### Comparison of MBV and EBV as Predictors of True BV

The ability of MBVs and BLUP EBVs to predict true BV was compared using a simple simulated example. The PCA was used to predict the MBV of the individuals in a simulated population where the true BVs were known for comparison. The data consisted of 1,000 SNPs, evenly spaced across the genome, with effects sampled from N(0, 1) and some regions were more favoured than others to give assumed differential gene locations across the genome. A heritability of 0.30 was used in both the simulation and BLUP analyses. A pedigree with approximately 1500 individuals was created.

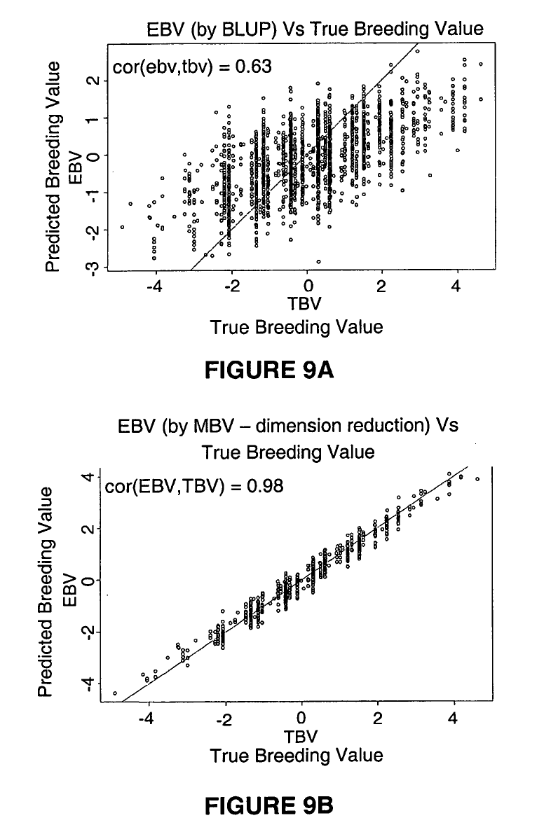

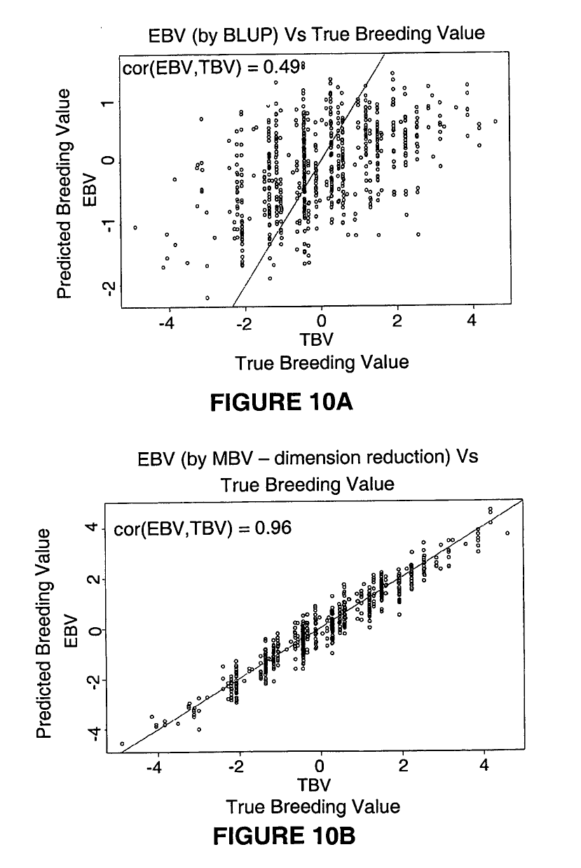

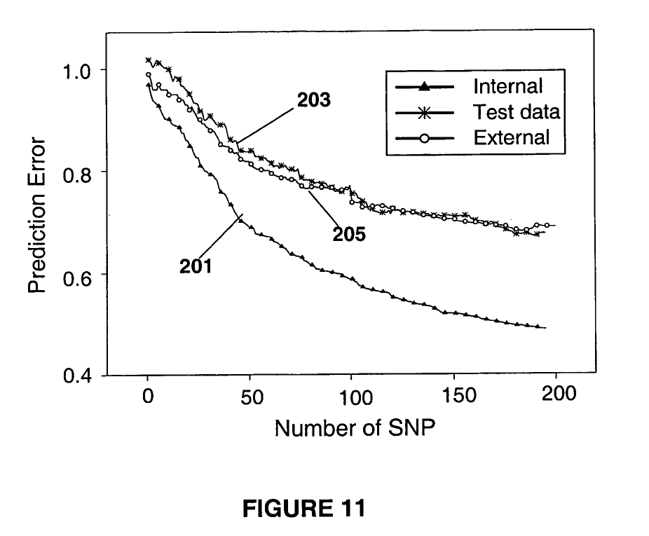

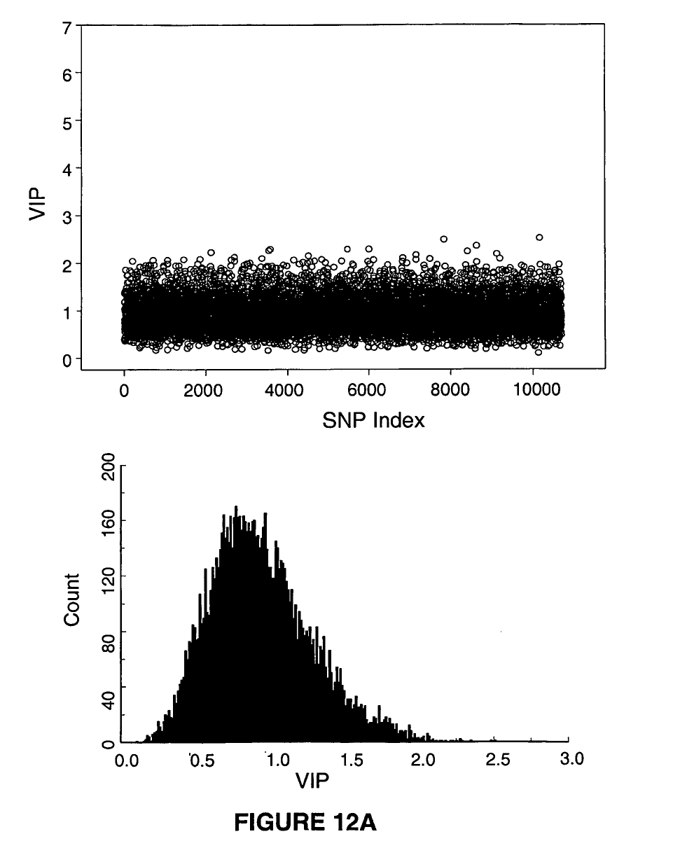

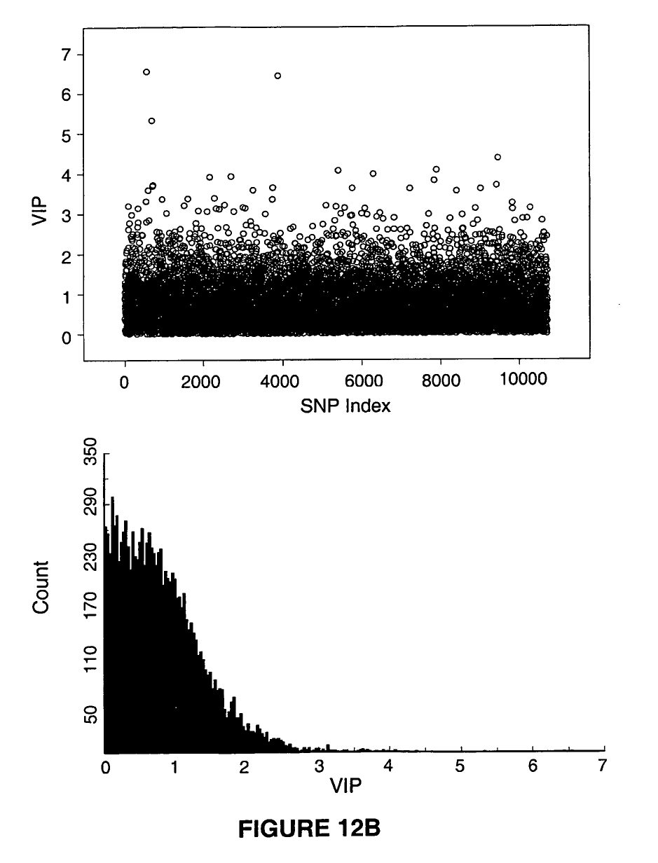

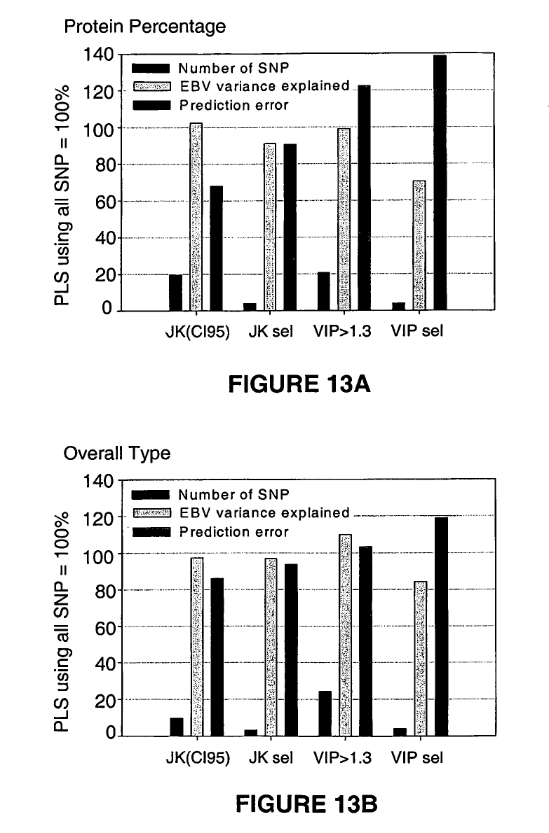

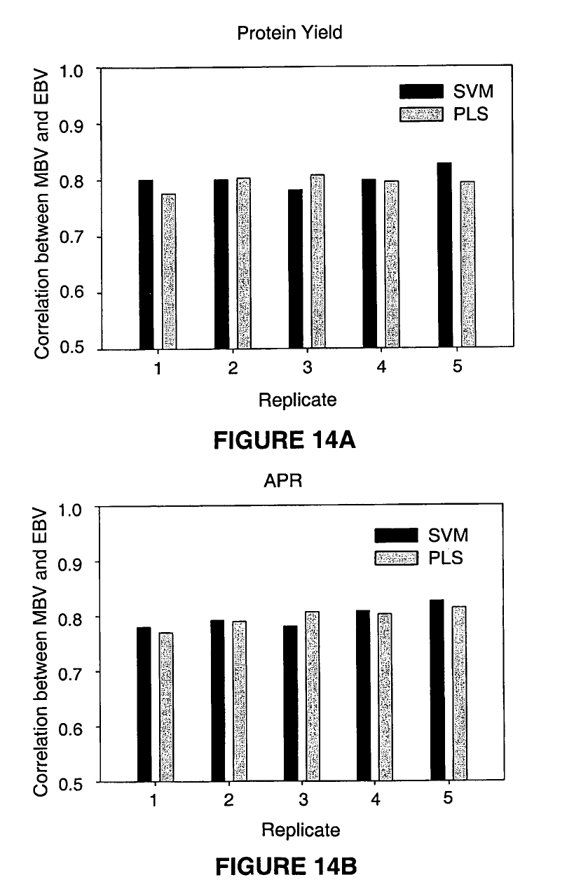

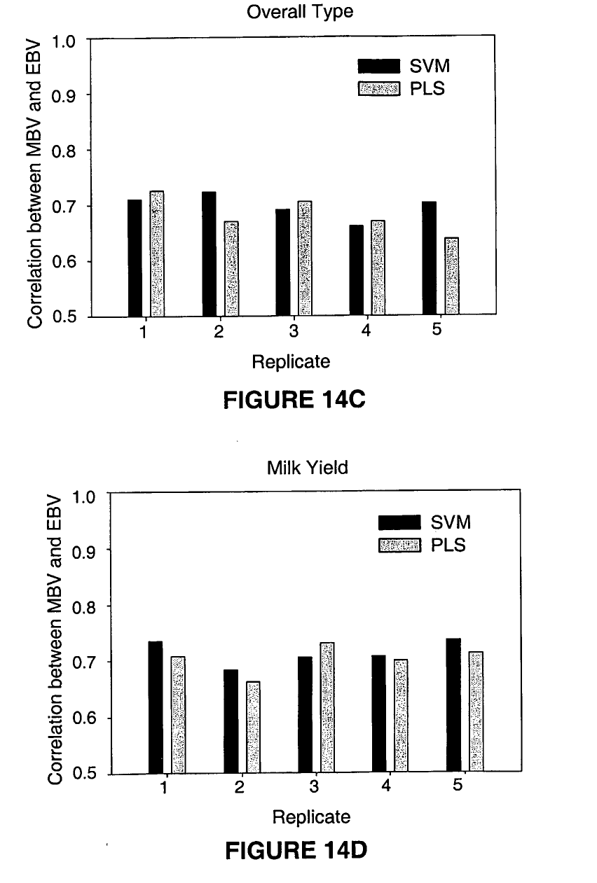

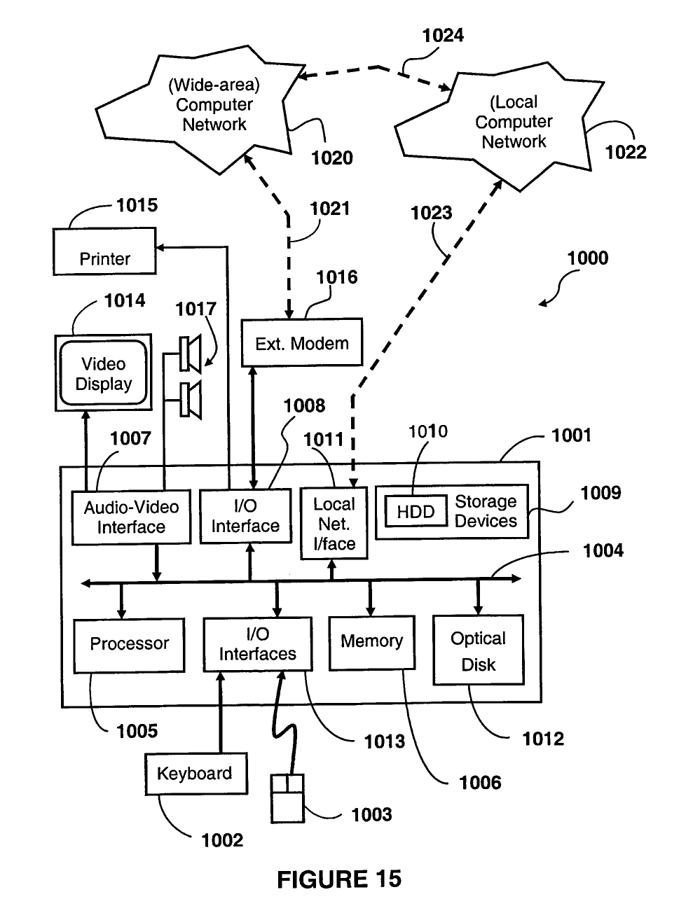

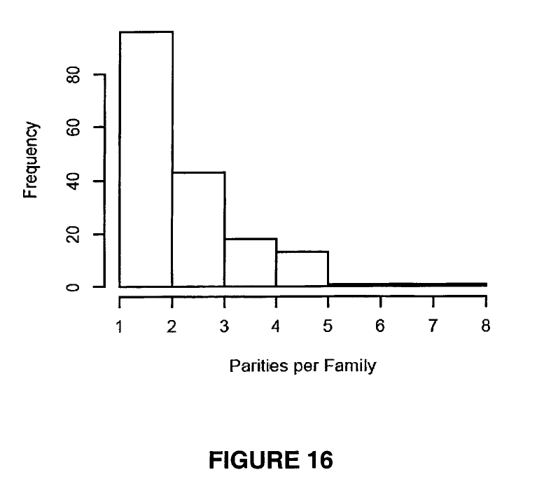

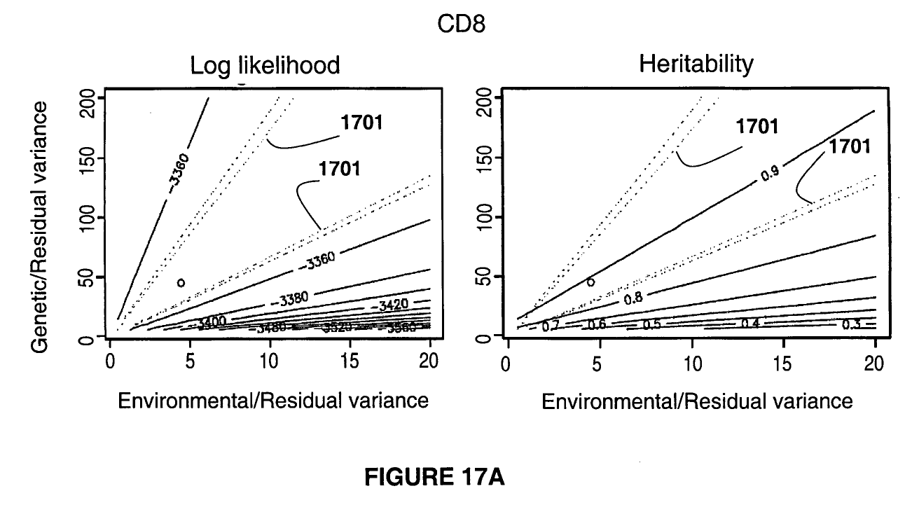

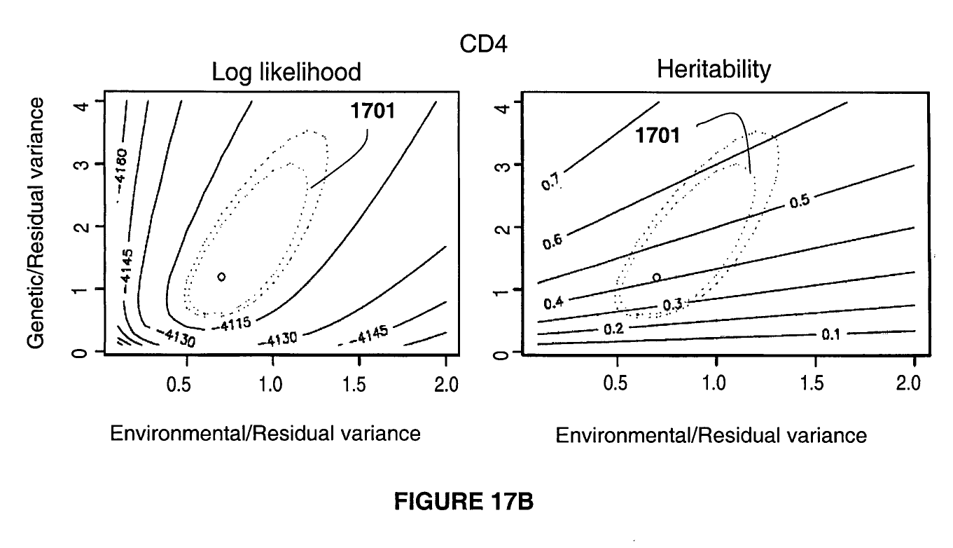

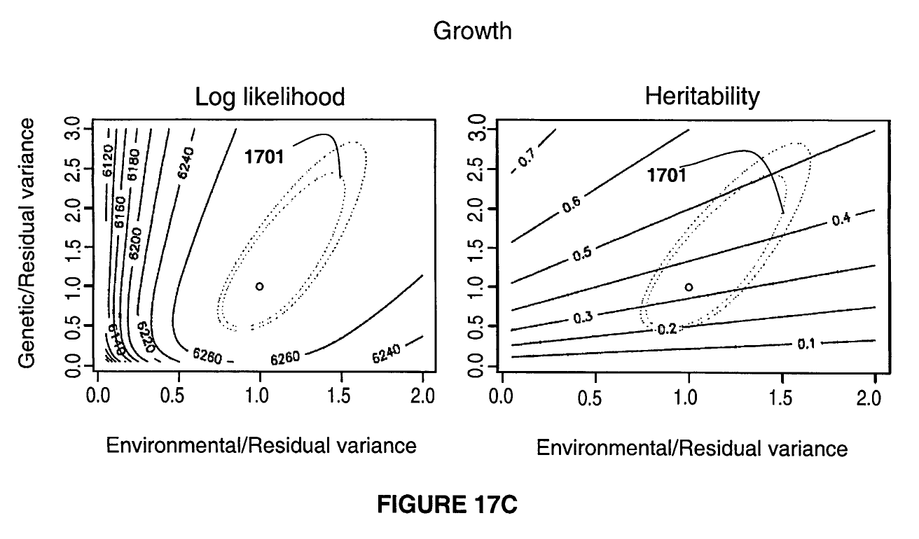

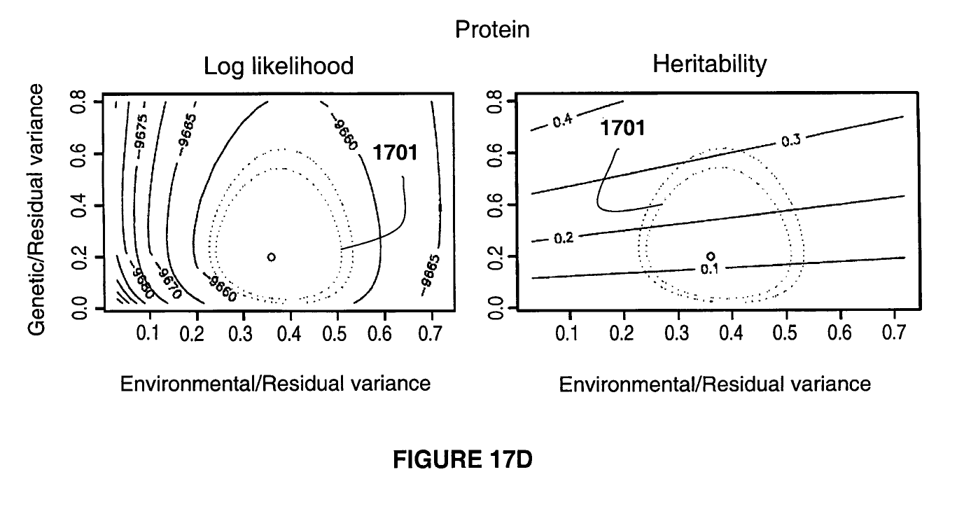

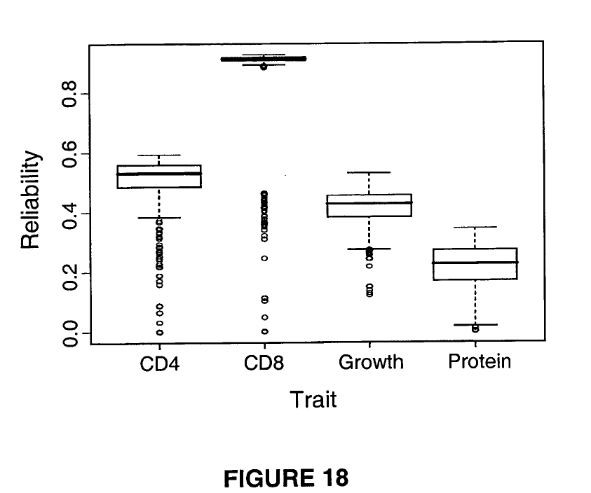

FIGS. 9 and 10 show the significant improvement of the MBV from the PCA for predicting the true breeding value of the individuals in the simple example compared with the commonly-used BLUP techniques over two generations.

FIG. 9A is a plot of the BLUP EBV for the simple example against the true BV as simulated, resulting in a correlation of 0.63. In comparison, FIG. 9B is a plot of the MBV for the simple example against the true BV as simulated, showing a significant improvement in the correlation to a value of r=0.98.

FIG. 10A is a plot of the BLUP EBV for the next generation of the simple example against the true BV as simulated. In this generation the correlation using the BLUP methods has deteriorated to only r=0.49. In comparison, FIG. 10B is a plot of the MBV of the next generation for the simple example against the true BV as simulated. In this case, the correlation is r=0.96 which is only a reduction of about 2%.

It is clear that calculation of MBVs provides a clear advantage over currently-used methods for prediction of BVs in a population across generations, at least for simple modes of inheritance.

### Example 5

### Partial Least Squares Analysis

Table 2 shows the results of PLS analysis for 38 indexes and traits of 1546 bulls using 10715 SNP. The proportion of the variance accounted for is shown for the PLS model of optimal complexity. The optimal complexity (i.e. number of latent components) was derived by 10-fold cross validation. A relatively small number of latent components (4-8) is required to account for a large proportion of the EBV variance (69%-94%). Less than 10% of the SNP variance is explained by the model, indicating a large proportion of redundant information in the marker data. The correlation between MBV and EBV is computed as the square root of the proportion of the explained EBV variance and lies between 0.82 and 0.97.

TABLE 2
Fit of PLS model for 38 indexes and traits of 1546 bulls using 10715 SNP

| | |
|---|---|
| | Proportion of |
| | variance |
| Number of latent | accounted for |

| Trait | components | EBV | Marker |
|---|---|---|---|
| APR | 6 | 91.64 | 7.06 |
| ASI | 6 | 90.95 | 7.13 |
| Protein kg | 7 | 94.07 | 7.60 |
| Protein % | 8 | 93.20 | 8.56 |
| milk | 7 | 91.70 | 7.69 |
| Fat kg | 5 | 81.86 | 6.34 |
| Fat % | 8 | 92.05 | 8.66 |
| Overall Type | 4 | 78.67 | 5.59 |
| Mammary System | 4 | 80.74 | 5.68 |
| Stature | 4 | 71.77 | 5.92 |
| Udder Texture | 4 | 79.24 | 5.97 |
| Bone Quality | 4 | 73.09 | 5.93 |
| Angularity | 4 | 69.54 | 5.76 |
| Muzzle Width | 5 | 79.86 | 6.70 |
| Body Depth | 6 | 85.83 | 7.19 |
| Chest Width | 5 | 79.31 | 6.63 |
| Pin Width | 5 | 78.39 | 6.57 |
| Pin Set | 4 | 70.50 | 5.65 |
| Foot Angle | 5 | 77.42 | 6.66 |
| Rearset | 5 | 80.11 | 6.43 |
| Rear Leg View | 4 | 66.65 | 5.87 |
| Udder Depth | 5 | 77.07 | 6.57 |
| Fore Attachment | 4 | 70.49 | 5.61 |
| Rear Attachment High | 4 | 77.69 | 5.84 |
| Rear Attachment Width | 4 | 75.18 | 5.82 |
| Centre Ligament | 4 | 77.10 | 5.77 |
| Teat Placement | 6 | 86.06 | 7.19 |
| Teat Length | 4 | 73.46 | 5.62 |
| Loin Strength | 4 | 74.40 | 5.67 |
| Milking Speed | 5 | 80.33 | 6.36 |
| Temperament | 5 | 80.91 | 6.35 |
| Likeability | 5 | 83.88 | 6.32 |
| Survival | 4 | 79.97 | 5.74 |
| Calving ease | 4 | 68.39 | 5.63 |
| Somatic Cell Count | 4 | 69.60 | 5.30 |
| Cow Fertility | 4 | 77.36 | 5.73 |
| Live Weight | 4 | 70.83 | 5.94 |

### Example 6

### PLS Model Validation

Table 3 shows the results of the validation of the PLS model for the Cow Fertility trait. The PLS model had 20 latent components and was first derived for the trait Cow Fertility using 1546 bulls and 10715 SNP (original data). The model fit was assessed by the coefficient of determination (R²). A prediction model (validation set) was computed based on 10-fold cross-validation. To test if high R² values for the original data are caused by overfitting (i.e. using a large number of SNP) the EBV of the original data were randomly assigned to animals (permuted data). This step was repeated 20 times. It can been seen from Table 3 that even for randomized data the PLS method fits the observations well, particularly if an increasing number of components is fitted in the model. However, these models show no predictive power. The high R² values in the prediction set of the original data demonstrate that the PLS method does not suffer from overfitting.

This is further reiterated by the results shown in FIG. 11, which show an example of the effect of prediction bias in SNP selection. The potential for inducing a bias in the SNP selection process can be shown for the trait APR. An external validation set of 200 bulls were randomly selected and excluded from the PLS analysis. The error curve 201 labelled “Internal” was estimated by cross-validation of models trained on subsets of increasing size, after the feature ranking was performed on all available data. The line 203 labelled “Test Data” shows the true prediction error when these internal validated models were used to predict MBV in the unseen test data. The reuse of information leads to optimistically biased estimates of the prediction error, suggesting that a small number of SNP can provide an accurate prediction of MBV. Using an external validation i.e. line 205 of FIG. 11 for performance assessment yields unbiased estimates of the prediction error.

TABLE 3
Validation of PLS model for Cow Fertility

| Number | R² in original data | | | |
|---|---|---|---|---|
| latent | Learning | | R² in permutated data | |
| components | set | Validation set | Learning set | Validation set |
| 1 | .51 | .58 | .20 | .005 |
| 2 | .67 | .65 | .36 | .008 |
| 3 | .76 | .67 | .50 | .007 |
| 4 | .84 | .70 | .62 | .007 |
| 5 | .89 | .70 | .70 | .006 |
| 6 | .92 | .68 | .77 | .006 |
| 7 | .94 | .67 | .82 | .006 |
| 8 | .96 | .67 | .86 | .007 |
| 9 | .97 | .66 | .89 | .007 |
| 10 | .97 | .66 | .91 | .008 |
| 11 | .98 | .66 | .93 | .008 |
| 12 | .98 | .65 | .94 | .008 |
| 13 | .99 | .64 | .96 | .008 |
| 14 | .99 | .63 | .97 | .008 |
| 15 | .99 | .63 | .97 | .009 |
| 16 | .99 | .63 | .98 | .009 |
| 17 | 1.00 | .62 | .98 | .009 |
| 18 | 1.00 | .62 | .99 | .009 |
| 19 | 1.00 | .62 | .99 | .009 |
| 20 | 1.00 | .61 | .99 | .010 |

### Example 7

### SNP Weight Distribution

FIGS. 12A and 12B show the VIP (variable importance in projection) distribution for the traits ASI and Overall Type, respectively. SNP with an average contribution to the model have a VIP value of equal 1. High values reflect the importance of the SNP in the PLS model both with respect to their correlation to the EBV and with respect to the SNP data. For both traits more than half of the SNP are of less than average importance. For the trait ASI less than 40 SNP have a VIP>2, compared with more than 400 for the trait Overall Type. Ranking SNP according to their VIP value allows identification of SNP that are useful in predicting breeding values.

### Example 8

### SNP Selection Process

FIGS. 13A and 13B show examples of the results from the SNP selection process for the traits Protein percentage (FIG. 13A) and Overall type (FIG. 13B). First a PLS analysis including all SNP(N=10715) was fitted. The number of SNP, the EBV variance explained and the prediction error of the model were set to equal 100% and compared to four different approaches of SNP selection. The first selection approach (JK (CI95)) was based on the jack-knife method, and all variables whose PLS regression coefficients have jack-knife confidence intervals (at the 95% level) that contain zero are eliminated at the same time. The set of SNP derived by JK (CI95) was used for a second SNP selection method in which individual SNP were selected by forward selection (JK sel). In the third model (VIP>1.3) only SNP with a VIP>1.3 were included in the PLS model. The fourth selection method was forward selection of SNP based on their VIP value (VIP sel). The SNP selection models were validated by 5-fold cross-validation. The results show that SNP selection methods are able to derive models with a predictive performance that is very similar to the model utilizing all SNP.

### Example 9

### Comparison Between PLS and Support Vector Machine Analysis

FIGS. 14A to 14D examine the predictive performance of the two supervised learning methods partial least squares (PLS) and support vector machines (SVM) using a radial basis function kernel. Five replicates were analysed for the four traits APR, Milk yield, Protein yield and Overall Type (FIGS. 14A to 14D respectively).

In each replicate 200 animals were randomly selected to form a test data set, which was not included in training the models. The test sets were chosen in a way that they do not overlap between replicates. PLS and SVM performed equally well in predicting molecular breeding value (MBV). For example for the five replicates of APR the correlation between MBV and EBV was in the range of 0.78 to 0.83 for both methods.

### Example 10

### Australian Profit Ranking (APR)

The Australian Profit Ranking (APR) is an index which uses ABVs to estimate a ranking that identifies those bulls that produce the most profitable daughters. ADHIS will continue to produce ABV's for all individual traits and the Australian Selection Index (ASI). This provides producers with the option to select on ASI or other combinations of traits.

The Australian Profit Ranking (APR)=Selection Index (ASI)+Milking Speed (MS)+Temperament (TEMP)+Survival (SURV)+Somatic Cell Count (SCC)+Live Weight (LWT)+Fertility (FERT), wherein each component is calculated as per the following:

ASI=(3.8×Protein ABV)+(0.9×Fat ABV)−(0.048×Milk ABV)

Milking Speed(MS)=1.2×(Milking Speed ABV)

Temperament(TEMP)=2.0×(Temperament ABV)

Survival(SURV)=3.9×(Survival ABV)

SCC=−0.34×(Somatic Cell Count ABV)

LWT=−0.26×(Liveweight ABV)

FERT=3.0×(Daughter Fertility ABV)

### Example 11

### Production Traits

Protein Yield (kg)

Protein content of milk is assessed in automated machines (Bentley Instruments www. Bentleigh instruments.com; Foss Instruments www.Foss.dk). Protein content of milk is assessed by infrared scanning of milk specific for N—H amine bond absorption.

Protein (w/v) (%)

Protein % is calculated by dividing protein yield (g) by milk volume litres (L) multiplied by 100.

Milk Volume (Litres)

A volumetric sample from an on-farm meter is weighed, and milk volume is calculated on the basis of the weight and average density of milk.

Fat Yield (kg)

Fat yield is assessed in automated machines (Bentley Instruments; Foss Instruments). Fat yield of milk is assessed by infrared scanning of milk specific for C═O and C—H groups.

Fat % (w/v)

Fat % is calculated by dividing fat yield (g) by milk volume litres (L) multiplied by 100

### Example 12

### Individual Type Traits

These traits include stature, udder texture, bone quality, angularity, muzzle width, body depth, chest width, pin set, pin width, foot angle, rear leg view, udder depth, fore attachment, rear attachment height, rear attachment width, centre ligament, teat placement, teat length and loin strength

### Stature

Stature is measured from the top of the spine in between the hips to the ground. The measurement is precise. The trait is measured on a linear scale of 1-9, and each point increase is 3 cm within the range listed below:

| | |
|---|---|
| 1 - Short | 1.30 Metres |
| 5 - Intermediate | 1.42 Metres |
| 9 - Tall | 1.54 Metres |

Udder Texture

This is a measure of the glandular milk-producing tissue in the udder emphasized by its collapsibility when milked, vein network and softness. Fibrous and fatty tissue in the udder restricts a dairy cow's ability to produce large quantities of milk. A prominent and distinctive vein network on the side of the udder is a reliable indicator of desirable texture. The trait is measured on a linear scale of 1-9, wherein:
1—Fleshy
9—Soft

Bone Quality

Bone quality is believed to be a reliable indicator of milking ability in a dairy cow. A flat bone is “dense”, and is more desirable in dairy compared with round or coarse bones which are associated with beef rather than dairy production. The trait is measured on a linear scale of 1-9, wherein:
1—Coarse bone
9—Flat bone

Angularity

Angularity is defined as the angle and openness of the ribs, combined with the flatness of bone in two year old heifers. Angle and open rib account for 80% of the weighting and bone quality accounts for 20%. The trait is scored on a scale of 1-9 wherein:
1-3: Non Angular—Lacks angularity, close ribs, coarse bone
4-6: Intermediate angle with open rib
7-9: Very angular open ribbed flat bone.

Muzzle Width

Muzzle width and openness of nostrils is a highly desirable trait in a country such as Australia where cattle frequently walk vast distances to access feed in extremely warm conditions. The trait is scored on a scale of 1-9, wherein:
1—Narrow muzzle
9—Wide Muzzle

Body Depth

Is the distance between the top of spine and the bottom of the barrel at the last rib—the deepest point. The trait is scored on a scale of 1-9 wherein:
1-3 shallow
4-6 intermediate
7-9 Deep

Chest Width

Chest width is measured from the inside surface between the front two legs. This trait is measured on a linear scale from 1-9, where each point is equal to 2 cm based on the range listed below as per (1-3) Narrow 13 cm, (4-6) Intermediate and (7-9) Wide 29 cm.

Pin Set

This trait is measured as the angle of the rump structure from hooks (hips) to pins on a linear scale of 1-9:

| | | |
|---|---|---|
| 1 - High Pins | (4 | cm) |
| 2 - | (2 | cm) |
| 3 - Level | (0 | cm) |
| 4 - Slight slope | (−2 | cm) |
| 5 - Intermediate | (−4 | cm) |
| 6 - | (−6 | cm) |
| 7 - | (−8 | cm) |
| 8 - | (−10 | cm) |
| 9 - Extreme Slope | (−12 | cm) |

Pin Width

This trait is calculated as the distance between the most posterior point of the pin bones, where 1=10 cm and 9=26 cm and every point between is calculated upon intermediate 2 cm lengths.
1-3: Narrow
4-6: Intermediate
7-9: Wide

Foot Angle

This trait is calculated as the angle at the front of the rear hoof measured from the floor of the hairline at the right hoof. This trait is measured on a linear scale from 1-9, where:
1-3: Very Low angle
4-6: Intermediate angle
7-9 Wide angle
   where 1=15 degrees, 5=45 degrees and 9=65 degrees

Rear Leg View

This trait is the direction of the feet when the animal is viewed from the rear.
1—Extreme toe out
5—Intermediate toe out
9—Parallel feet

Udder Depth

This trait is calculated as the distance from the lowest part of the udder floor to the hock where:
1—Below hock
2—Level with hock
5—Intermediate
9—Shallow

Fore Udder Attachment

This trait is calculated as the strength of the attachment of the fore udder to the abdominal wall. This is not a true linear trait.
1-3: Weak and Loose
4-6: Intermediate acceptable
7-9: Extremely strong and light

Rear (Udder) Attachment Height

This trait is calculated as the distance between the bottom of the vulva and the milk secreting organ in relation to the height of the animal. A score of 4 represents the mid point of 29 cm, and each point is worth 2 cm.

| | | |
|---|---|---|
| 1 | Very Low | 23 cm |
| 2 | — | 25 cm |
| 3 | — | 27 cm |
| 4 | Intermediate | 29 cm |
| 5 | — | 31 cm |
| 6 | — | 33 cm |
| 7 | — | 35 cm |
| 8 | — | 37 cm |
| 9 | High | 39 cm |

Rear (Udder) Attachment Width

This trait is calculated wherein the reference point for measurement is the top of the milk secreting organ to each pin measured on a linear scale of 1 to 9, where 1 is extremely narrow and 9 is extremely wide.

Central Ligament

This trait is calculated as the depth of the cleft measured at the base of the rear udder.

| | | |
|---|---|---|
| 1 | Convex to flat floor | (1 cm) |
| 2 | — | (0.5 cm) |
| 3 | — | (0 cm) |
| 4 | Slight Definition | (−1 cm) |
| 5 | — | (−2 cm) |
| 6 | — | (−3 cm) |
| 7 | Deep Definition | (−4 cm) |
| 8 | — | (−5 cm) |
| 9 | — | (−6 cm) |

Teat Placement

This trait is calculated as the position of the front teat from the centre of the quarter.
1-3: Outside of quarter
4-6: Middle of quarter
7-9: Inside quarter

Teat Length

This trait is calculated as the length of the front teat, where each point is 1 cm and the scale ranges from 1 to 9.
1-3: Short
4-6: Intermediate
7-9: Long

### Example 13

### Live Weight

Live Weight is reported as a deviation in kilograms of live weight from the base set at zero. Live Weight is based on ABVs measured by breed societies. The predictors and their relative contributions are:

Live Weight=(0.5×stature ABV)+(0.25×Chest Width)+(0.25×Body Depth)

### Example 14

### Workability

Workability is reported as a combination of the following traits: milking speed, temperament and likeability.

Each of these traits is scored on a scale from A to E by the dairy farmer, where A is very desirable and E is very undesirable. Satisfactory daughters are those expected to receive scores of C, B or A from the farmer. The metric is expressed as a percentage:

$$\frac{\% = number of offspring expected to be satisfactory ( A , B , C )}{all offspring ranked} \times 100$$

### Example 15

### Somatic Cell Count

Somatic cell count breeding value is expressed as the % increase or decrease in cell count compared to the average or BASE (i.e. the average count is scored as a zero percentage deviation). Thus a bull with lower SCC ABV has daughters with lower somatic cell count which is an indicator of increased mastitis resistance, and a bull with a higher SCC ABV has daughters with higher somatic cell count which is an indicator of mastitis susceptibility.

Somatic cell count can be assessed by laser-based flow cytometry, which is a common method for distinguishing between different cell populations and/or counting cell numbers. Briefly, a milk sample is taken and mixed with a fluorescent dye, which disperses the globules and stains DNA in somatic cells. An aliquot of the stained suspension is injected into a laminar stream of carrier fluid. Somatic cells are separated by the stream of carrier fluid and exposed to a laser beam. As the cells pass through the excitation source the stained cell nuclei fluoresce, the signal is multiplied and cell number calculated. Indicative SCC levels are as follows:
Over 200,000: mastitis
<200,000: maximum desired number of somatic cells/ml milk
<100,000: number of somatic cells/ml milk where the cow is considered to have minimal to no mastitis [ICAR]

### Example 17

### Fertility

Daughter fertility is a measurement of the difference between bulls for the percentage of their daughters pregnant by 6 weeks after mating start date. In year-round herds this is equivalent to the percentage of their daughters pregnant by 100 days after calving. Data is derived from the following records:
Calving dates used to determine calving interval and stage of pregnancy
Mating data is used to determine days to first service

### Example 18

### Survival

The survival index is reported as the percentage of daughters that survive from one year to the next compared to the average/BASE (set at zero). The Survival Index is based on actual daughter survival and a combination of predictors of survival. The predictors and their relative contributions are:

Survival Predictors=(0.5×likeability)+(1.8×Overall Type)+(3.0×Udder Depth)+(2.2×Pin Set)

### Example 19

### Calving Ease

The calving ease is expressed as the percentage of ‘normal’ carvings expected when joined to mature cows in the average Australian herd. The calving ease for a bull is based on farmer assessment of the difficulty experienced with the birth of the progeny of the bull, relative to births in the same herd in the same season.

### Example 20

### Mammary System

Mammary System ABV is calculated using the formula below based on linear traits that have been differentially weighted. The differential weighting of each of the linear traits is based on regression analysis and the contribution of these traits to the variance observed in the system overall.

Mammary System=(Udder texture×0.161)+(Fore Attachment×0.4753)+(Rear attachment height×0.454)+(rear attachment width×0.448)+(Centre Ligament×0.355)+(teat placement×0.269)

### Example 21

### Overall Type

Overall type is a categorisation of an individual assigned by a person skilled in the art on the basis of an assessment of “type” traits individually assessed.

### Example 22

### Selection Index

Selection Index is expressed as the net financial profit (in $) per cow per year. It includes a consideration of protein, fat and milk volume traits. The formulation is based on the milk payment system whereby farmers are paid by the amounts of protein and fat in milk, with a charge on milk volume:

ASI=(3.8×Protein Yield ABV)+(0.9×Fat Yield ABV)−(0.048×Milk Volume ABV)

### Example 23

### Lactation Traits

Lactation traits can also be used in predicting the genetic merit of an animal.

A lactation curve is the graph of milk production against time. Each cow in a herd has its own individual curve relating to its lactation potential and other external influences such as the environment and nutrition. Characteristics of the curve include measurements such as the persistency of lactation, total milk produced over the lactation, and the time of peak production.

Wood proposed the following function to model the lactation curve W(t)=at^{b}e^{-ct} where W(t) is the theoretical or expected milk yield at time t; and a, b, and c are parameters which determine the shape of the curve (Wood et al. 1967). The parameters of the Wood function have been reparameterised to obtain estimates for total volume, peak volume and time to reach the peak.

Negative energy balance in early lactation is often associated with reduced fertility. This is usually a result of the cow producing at her peak at the time of insemination. A cow with a low peak and consistent production should be able to avoid these problems and maintain fertility. These cows can now be identified with the assistance of the estimates from the model.

Another application of the model is prediction of lactation potential from the first few records, which would allow farmers to manage their herds appropriately in terms of feeding and reproduction (an example list of common lactation traits and corresponding variables of importance for each trait is provided in Table 4).

TABLE 4
List of Lactation Traits

| Category | No. | Parameters* | Variable Names |
|---|---|---|---|
| Wood Model | 1. | LogA | LogA |
| | 2. | B | B |
| | 3. | C | C |
| Persistency | 4. | Proportion of Ytot attained by 300 days | P(300) |
| | 5. | Ratio of peak yield to yield on Day 300 | yₘₐₓ:y(300) |
| Yield | 6. | Cumulative yield up to 300 days (=Yₜₒₜ * P(300)) | YCum(300) |
| | 7. | Total cumulative Yield | Ytot |
| | 8. | Maximum Yield | Ymax |
| | 9. | Time of maximum yield | tmax |
| | 10. | Milk yield at Day 300 (not cumulative) | Y300 |
| | 11. | Time at which 90% of Yₜₒₜ is reached | t(0.9Ytot) |
| | 12. | Extrapolation measure for for t(0.9Yₜₒₜ): 1 if extrapolation (after recording | X(0.9Yₜₒₜ) |
| | | stopped), 0 otherwise | |
| | 13. | Time at which 75% of Yₜₒₜ is reached | t(0.75Ytot) |
| | 14. | Extrapolation measure for t(0.9Yₜₒₜ ): 1 if extrapolation (after recording | X(0.75Yₜₒₜ) |
| | | stopped), 0 otherwise | |

*Original Parameter: No. 1-3; Derived Parameter: No. 4-14

### Example 24

### Application to Other Animals and Species

Whole genome-wide marker information is available for humans, many other species of mammals, several non-mammalian vertebrate species, some fish, and many plants. As a first step, whole genome marker information can be generated using one of several genotyping systems which are commercially available (e.g. from Illumina, San Diego, Calif.). Accordingly, using the methods described above, SNP information is associated with the trait, thereby inferring the trait. The SNPs can comprise all marker data, or a limited set of markers may be inferred. Where the trait is a health condition, the outcome may be inferring the risk that an individual will pass on the condition to its offspring. The methods disclosed herein also enable persons skilled in the art to develop a set of diagnostic SNPs and genetic profiling tools for assessing the likelihood that an individual will have a specific characteristic. This includes:

the risk that an individual will develop a disease or condition, such as diabetes, heart disease etc;

the risk that an individual will develop an adverse reaction to a specific pharmaceutical agent;

predictions regarding productivity, e.g. for livestock animals; and

predictions regarding athletic performance, e.g. for human athletes and sportspeople or for racing animals.

A whole-genome association study can be undertaken in a number of ways, depending on the number of animals and the number of traits under study. The population structure can be of several types. The situation in the case of animals with high reproductive rate differs considerably from that with large animals, which generally have a low reproductive rate. Differences also exist between individual animals within a species. For example, in chickens an exemplary strategy may comprise producing 1000 progeny from 10 sires, mated to 2000 dams, with half-sib groups of 50 progeny per sire. In this case highly accurate breeding values can be computed from the progeny means. Other designs are possible, depending upon the use to which the results will be put.

For example, Zebaneh and Mackay (2003) computed breeding values for the trait fasting triglyceride level using data studied at the Genetic Analysis Workshop 13. Their method was similar to other methods which used adjusted phenotypes of various forms.

Therefore the methods of the invention can be applied to this type of analysis, and are not limited to breeding value information, but are applicable to trait information of any kind.

Many analyses of human genomic information to identify markers for disease susceptibility have been performed. For example markers for multiple sclerosis and for endometriosis have been identified. The methods of the invention may be applied to this type of analysis.

The population structure can be of several types. The situation in the case of animals with high reproductive rate differs considerably from that with large animals, which generally have a low reproductive rate. Differences also exist between individual animals within a species. In chickens an exemplary strategy may comprise producing 1000 progeny from 10 sires, mated to 2000 dams, with half-sib groups of 50 progeny per sire. In this case highly accurate breeding values can be computed from the progeny means. Other designs are possible, depending upon the use to which the results will be put.

A whole-genome association study can be undertaken in a number of ways, depending on the number of animals and the number of traits under study. The simplest analysis is least-squares regression on every marker. However, a serious problem with this approach is overestimation of the SNP effects. Therefore several methods which analyse several linked marker or haplotypes have been developed. These methods use either linkage or linkage disequilibrium information, or a combination of the two (Meuwissen et al, 2002), which requires prior information about the location and the distances between SNP. In contrast to prior art methods, a powerful feature of the invention is that the phenotypic merit of individuals can be assessed without the need for comprehensive and annotated genome information in a species, which may not be available at the time of analysis.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

### Example 25

### Application to Mouse Data

The following example show the application of the methods described above to genotype and phenotype data in mice. The data used in the present example was sourced from http://gscan.well.ox.ac.uk and include phenotypic and genotypic measures for 2296 mice from 4 generations. A total of 12112 SNPs are genotyped for each mouse, but some are missing genotypic scores. The heterogenous stock mice are a result of 50 generations of breeding between 8 inbred families. The first generation of phenotyped mice in these data are defined as mice with unknown parents. The generation number of mice in subsequent generations is defined as the maximum generation of the parents plus 1. Table 5 displays the total mice in the pedigree (n), mice with more than 11112 recorded SNPs (n_{geno}), and the number of full sib families in each generation (n_{fams}).

TABLE 5
Number of mice per generation

| Generation | n | n_{geno} | n_{fams} |
|---|---|---|---|
| 1 | 258 | 155 | — |
| 2 | 1019 | 1016 | 113 |
| 3 | 558 | 558 | 36 |
| 4 | 461 | 461 | 33 |
| All | 2296 | 2190 | 182 |

The families in table 1 are defined to be full sib families and each family may be comprised of more than one parity. The distribution of the number of parities per family is displayed in FIG. 16.

Same sex litter mates were housed together in cages. Only a small number of cages contained more than one litter, as displayed in Table 6. This experimental design makes the environmental cage effects and the genetic effects almost completely confounded. This is illustrated by the small effective population size for each trait, defined as

$${n}_{ef} = {\sum }_{j} {\sum }_{i} \frac{{n}_{ij} ( {η}_{j} - {n}_{ij} )}{{η}_{j}} ,$$

where nᵢⱼ is the number of mice in family i, cage j and nⱼ is the number of mice in the j^{th} cage. Similarly, sex effects cannot be separated from cage effects.
Table 6: Number of individuals, families and cages with phenotypic records for selected traits.

| | | | | | Families | | | Cages | | |
|---|---|---|---|---|---|---|---|---|---|---|
| | All records | | | | in > 2 cages | | | with > 1 family | | |
| Trait | n_{nind} | n_{fam} | n_{cage} | n_{ef} | n_{nind} | n_{fam} | n_{cage} | n_{nind} | n_{fam} | n_{cage} |
| CD8% | 1869 | 166 | 450 | 41.8 | 1367 | 76 | 328 | 57 | 23 | 14 |
| CD4/CD8 | 1864 | 166 | 449 | 41.4 | 1363 | 76 | 327 | 56 | 23 | 14 |
| CD4/CD3 | 1868 | 166 | 450 | 41.8 | 1366 | 76 | 328 | 57 | 23 | 14 |
| B220% | 1858 | 164 | 440 | 41.8 | 1329 | 72 | 315 | 57 | 23 | 14 |
| CD3% | 1869 | 166 | 450 | 41.8 | 1366 | 76 | 328 | 57 | 23 | 14 |
| CD4% | 1867 | 166 | 450 | 41.8 | 1365 | 76 | 328 | 57 | 23 | 14 |
| Albumin | 1945 | 175 | 525 | 62.8 | 1560 | 97 | 420 | 73 | 30 | 19 |
| Calcium | 1945 | 176 | 521 | 52.8 | 1558 | 97 | 417 | 74 | 32 | 30 |
| Glucose | 1905 | 176 | 527 | 44.4 | 1521 | 97 | 422 | 69 | 30 | 18 |
| Protein | 1832 | 176 | 502 | 47.1 | 1414 | 92 | 388 | 75 | 31 | 19 |
| Urea | 1945 | 176 | 518 | 56.1 | 1558 | 98 | 415 | 79 | 32 | 20 |
| Start Weight | 2511 | 180 | 552 | 75.9 | 2040 | 102 | 449 | 107 | 35 | 23 |
| End Weight | 2320 | 177 | 541 | 64.8 | 1888 | 101 | 439 | 98 | 35 | 23 |
| Growth | 2474 | 180 | 500 | 65.7 | 1997 | 101 | 446 | 101 | 35 | 22 |
| Hematocrit | 1888 | 160 | 458 | 30.2 | 1458 | 79 | 350 | 42 | 19 | 12 |
| RBC | 1885 | 160 | 458 | 29.7 | 1456 | 79 | 350 | 41 | 19 | 12 |

Variance Components

Valdar et al. (2006) give the heritabilities and variance due to environment for a variety of traits for all animals with phenotypic records. Some of these heritabilies are recalculated here for mice with both genotypic and phenotypic information and are displayed in table 3. The model used is as in Valdar et al. (2006):

Let yᵢⱼ∈G be the phenotype of the i^{th} animal in cage j, μ be the grand mean, dⱼ be the random effect of cage j, aᵢⱼ be the animal's additive genetic random effect, xᵢⱼ(c) be its value for covariate c, β_{c} be the covariate associated with fixed effect c, C be the set of fixed effect covariates and eᵢⱼ the random effect of uncorrelated noise. Then

$${y}_{ij} = μ + {\sum }_{c ∈ C} {β}_{c} {x}_{ij} ( c ) + {d}_{j} + {a}_{ij} + {e}_{ijk} \qquad ( 4 )$$

where e˜N(0,σ_{E}²I), d˜N(0,σ_{P}²I), a˜N(0,σ_{A}²A) and A is the genetic relationship matrix. Normalizing transformations are applied to the phenotypes using the transformations as described in Valdar et al. (2006) for each trait. The set of fixed effects (C) is comprised of age, cage density, litter, weight (continuous), month, sex, experimenter and year (categorical).

TABLE 7
Variance components and their approximate standard errors

| Phenotype | n | σₚ² | σₐ² | σ_{c}² | h² | σ_{c}²/σₚ² |
|---|---|---|---|---|---|---|
| CD8% | 1521 | 21.55 (1.42) | 19.25 (2.79) | 0.38 (1.45) | 0.89 (0.08) | 0.09 (0.02) |
| CD4/CD8 | 1516 | 2.23 (0.15) | 1.90 (0.29) | 0.26 (0.05) | 0.83 (0.08) | 0.10 (0.02) |
| (×10−2) | | | | | | |
| CD4/CD3 | 1520 | 7.49 (0.48) | 5.95 (0.94) | 0.84 (0.15) | 0.79 (0.08) | 0.11 (0.02) |
| (×105) | | | | | | |
| B220% | 1522 | 82.90 (4.84) | 48.97 (9.28) | 19.11 (2.50) | 0.59 (0.09) | 0.23 (0.03) |
| CD3% | 1521 | 1.13 (0.06) | 0.53 (0.11) | 0.27 (0.35) | 0.47 (0.08) | 0.27 (0.03) |
| (×108) | | | | | | |
| CD4% | 1520 | 48.64 (2.47) | 20.09 (4.43) | 12.13 (1.61) | 0.41 (0.08) | 0.25 (0.03) |
| Albumin | 1744 | 6.39 (0.26) | 0.92 (0.36) | 1.20 (0.21) | 0.14 (0.05) | 0.19 (0.03) |
| (g/liter) | | | | | | |
| Calcium | 1751 | 2.72 (0.12) | 0.37 (0.18) | 0.81 (0.11) | 0.14 (0.06) | 0.30 (0.04) |
| (mmol × 10-2) | | | | | | |
| Glucose | 1705 | 2022 (92) | 444 (146) | 554 (77) | 0.22 (0.07) | 0.27 (0.03) |
| Protein (×105) | 1640 | 1.48 (0.06) | 0.19 (0.09) | 0.34 (0.06) | 0.13 (0.06) | 0.23 (0.03) |
| Urea (×10-2) | 1743 | 3.06 (0.14) | 0.87 (0.22) | 0.64 (0.10) | 0.28 (0.07) | 0.21 (0.03) |
| Start Weight | 1928 | 2.29 (0.07) | 1.69 (0.05) | 0.60 (0.02) | 0.73 (0.09) | 0.26 (0.03) |
| (×10-1) | | | | | | |
| End Weight | 1884 | 1.43 (0.07) | 0.87 (0.14) | 0.25 (0.03) | 0.61 (0.07) | 0.17 (0.02) |
| (×10-2) | | | | | | |
| Growth Slope | 1920 | 2.72 (0.12) | 0.92 (0.21) | 0.91 (0.09) | 0.34 (0.07) | 0.33 (0.03) |
| (×10-3) | | | | | | |
| Hematocrit | 1593 | 2.11 (0.08) | 0.22 (0.10) | 0.44 (0.07) | 0.10 (0.05) | 0.21 (0.03) |
| (%) (×108) | | | | | | |
| Red blood cell | 1590 | 2.38 (0.09) | 0.32 (0.12) | 0.48 (0.07) | 0.13 (0.05) | 0.20 (0.03) |
| count (×104) | | | | | | |

Table 7 shows the variance components and their approximate standard errors wherein is the number of individuals with a record for the trait, σ_{P}² is the phenotypic variance, σₐ² is the additive genetic variance, σ_{c}² is the environmental variance due to the random cage effect and h² is the heritability. All of the heritability and σ_{c}²/σ_{P}² values in Table 7 are not significantly different to those displayed in Valdar et al. (2006), with the exception of Calcium, which they report to be 0.49 and 0.31 respectively.

It should be noted, however, that due to the confounding between cage and genetic effects and consequently the low effective population number, the maximum likelihood estimates of the variance parameters in Table 7 are unreliable. This is supported by the log-likelihood plots displayed in FIG. 17, which show the Log-likelihood contours for CD8, CD4, growth and protein (LHS) and corresponding heritability plots (RHS). Dotted contours represent the 10% and 5% thresholds from the LRT. These plots show the contours as the additive genetic and cage variances change. The inner dotted contours 1701 on each plot is a 10% significance region for the variance parameters (the outer dotted contours represent a 5% significance region for the variance parameters). This significance threshold is obtained by applying the likelihood ratio test (LRT) to the maximum log-likelihood value (ln(Lₘ)) for each trait. That is, for a point with log-likelihood ln(L₁), the ratio LR is defined as:

LR=2(ln(Lₘ)−ln(L₁))

which approximately follows a χ² distribution.

The log-likelihood plot for CD8 is particularly flat and the confidence region for the variance parameters is particularly large. Any heritability between 0.75 and 1 is feasible for CD8. Similarly for CD4, growth and protein, there is a large range of heritabilities that these data support.

Genome Wide Selection—Description of Phenotypes for GWS

Five variations of phenotype were created:
Raw: Raw phenotypes are predicted from genotypes only.
Cage: Phenotypes are adjusted for fixed effects including cage i.e. y_{cage}=y_{raw}−Σ_{o∈D}β_{c}x(c), where D is the set of fixed effects including cage.

Adjusted: Phenotypes are adjusted for fixed effects excluding cage i.e. y_{adj}=y_{raw}−Σ_{o∈C}β_{c}x(c), where C is the set of fixed effects excluding cage.
Adjusted_{cf}: Phenotypes are adjusted for the cage.family interaction i.e. y_{acf}=y_{raw}−βᵢ(cage, family)ᵢ.
EBV: EBVs from animal model described in Equation (4). The reliability of these EBVs is displayed in FIG. 18. Most of the animals unreliable EBVs have missing phenotypic information so that the EBV is calculated from the animal's relations.

Partial least squares (PLS) was applied to all of these phenotypes with the genotypic information acting as the predictor functions. In addition, PLS was applied to the raw data with both the SNPs and fixed effects excluding cage (sex, age, month, etc.) as explanatory variables (raw 2).

Forward Prediction

The data are divided into a training set comprised of all animals in the first 3 generations and a test set comprised of all animals in the last generation. PLS was applied to the test set and the resultant parameters are used to predict phenotypes for the test set. The correlation between the predicted phenotype and actual phenotype is displayed in Table 8.

TABLE 8
Forward prediction-PLS.

| Trait | Raw | Raw 2 | Cage | Adjusted | Adjusted_{cf} | EBVs |
|---|---|---|---|---|---|---|
| CD8 | 0.421 | 0.423 | 0.272 | 0.3766 | 0.265 | 0.434 |
| CD4 | 0.282 | 0.281 | 0.167 | 0.300 | 0.161 | 0.286 |
| Growth | 0.206 | 0.208 | 0.023 | 0.197 | 0.088 | 0.520 |
| Protein | 0.112 | 0.181- | 0.002 | 0.166- | 0.001 | 0.574 |

The accuracy of prediction is highest for the EBV phenotype for CD8, growth and protein. The adjusted phenotype yields the most accurate result for CD4. This would suggest that adding the pedigree information is advantageous. There is a large decline in accuracy when cage effects are corrected for as a fixed effect, with the accuracy of prediction for the ‘adjusted’ phenotype significantly higher than both the ‘cage’ and ‘adjusted_{cf}’ phenotypes. This is further evidence that cage effects and genetic effects are confounded.

Fitting fixed effects in the PLS model does little to improve the prediction accuracy for the raw data for CD8, CD4 and growth. This is probably caused by some SNPs being confounded with the fixed effects in the training set due to random sampling. However, there is a large improvement in accuracy for protein.

Mirror Test Set Prediction

The data are randomly divided into a test set of 300 mice and the remaining mice form the training set. As before, PLS is applied to the test set and the resultant parameters are used to predict phenotypes for the test set. This process is repeated 50 times for each trait and phenotype. The mean correlation and the standard deviation between the predicted phenotype to and actual phenotype for the 50 replications is displayed in Table 9.

TABLE 9
Mirror prediction-PLS. Mean and SD of 50 replicates.

| Trait | Raw | Raw 2 | Cage | Adjusted | Adjusted_{cf} | EBVs |
|---|---|---|---|---|---|---|
| CD8 | 0.689 (0.030) | 0.690 (0.030) | 0.236 (0.053) | 0.688 (0.031) | 0.235 (0.053) | 0.723 (0.028) |
| CD4 | 0.452 (0.043) | 0.453 (0.043) | 0.099 (0.044) | 0.444 (0.045) | 0.098 (0.042) | 0.738 (0.026) |
| Growth | 0.078 (0.049) | 0.148 (0.041) | 0.040 (0.050) | 0.114 (0.055) | 0.045 (0.050) | 0.152 (0.060) |
| Protein | 0.158 (0.048) | 0.273 (0.046) | −0.077 (0.047) | 0.173 (0.048) | −0.071 (0.057) | 0.737 (0.027) |

The accuracies for mirror set prediction are generally higher than accuracies for forward prediction. In the mirror prediction case, animals in the same cage can be used in the training and test sets, so that the confounding of environmental and genetic effects has less influence. In the forward prediction set, fitting cage as a fixed effect has a large negative effect on accuracy due to the experimental design.

The ‘EBVs’ phenotype has the best accuracy of prediction when PLS is applied for all 4 traits, with CD8, CD4 and protein having accuracies around 0.73. However the accuracy for growth is significantly lower (0.152).

### Example 25

### Application to Human Data

The applicability of the whole genome analysis approach using partial least squares (PLS) and support vector machines (SVM) were tested on two human data sets with the aim to identify genetic predictors associated with increased or decreased risk for developing a particular disease (Parkinson's disease and amyotrophic lateral sclerosis, ALS). A description of the data is given below (Table 10). All DNA samples and raw genotype data are publicly available. The authors of both studies analysed the data by testing each SNP individually and both studies were unable to detect common genetic variants that exert an significant effect.

TABLE 10
Description of Parkinson's disease and ALS data sets

| | Cases | Control | SNP | Reference |
|---|---|---|---|---|
| Parkinson's disease | 270 | 271 | 389 879 | Fung et al. Lancet Neurol |
| | | | | 2006; 5: 911-16 |
| ALS | 276 | 271 | 503 875 | Schymick et al., Lancet |
| | | | | Neurol 2007; 6: 322-28 |

SVM and PLS gave very similar results and we only report details of the PLS here. Briefly, a PLS analysis was performed in the following steps:
1. Imputation of missing genotypes using the NIPALS algorithm
2. Splitting the data in validation and test set. The test set included 10 randomly selected cases and 10 randomly selected controls.
3. SNP selection by 10-fold external cross-validation using a 95% jackknife confidence interval

The results are reported in form of the classification error and the number of selected SNP (Table 11). In a random data set we would expect an classification error of 50%. The final prediction model build with PLS results in smaller classification errors for both diseases, however the error is magnitudes too large for the model to have any utility as an disease diagnostic. Overall, the analyses confirm the findings of the original studies, that neither for Parkinson's disease nor for ALS common genetic variants of larger effects can be identified. The authors of the studies discuss several reasons for the lack of associations between markers and disease risk (e.g. limited power because of sample size and age-matched and sex-matched controls, sporadic ALS may consist of diverse group of clinically indistinguishable genetic disorders, etc.)

TABLE 11
Results of partial least squares analysis (PLS)
for Parkinson's disease and ALS

| | SNP | Classification error |
|---|---|---|
| Parkinson's disease | 11 854 | 0.25 |
| ALS | 14 891 | 0.33 |

To increase the statistical power of the study would require to whole-genome scan additional patients and control. However, it may be cost-effective to do follow-up genotyping of only the 3% of SNP markers identified by the whole-genome PLS analysis.

It will be appreciated that the methods and systems described above at least substantially provide a significantly improved genome based selection process.

The systems and processes described herein, and/or shown in the drawings, are presented by way of example only and are not limiting as to the scope of the described methods. Unless otherwise specifically stated, individual aspects and components of the processes may be modified, or may have been substituted, therefore equivalents, or as yet unknown substitutes such as may be developed in the future or such as may be found to be acceptable substitutes in the future. The processes may also be modified for a variety of applications while remaining within the scope and spirit of the claimed invention, since the range of potential applications is great, and since it is intended that the present processes be adaptable to many such variations.

### Example 26

### Genetic Algorithm on Beef Data Set

The present example demonstrates a phenotype predictor using SNP identification of phenotype based on MBV as biomarker and highlights three applications of the above methods:

a) GA-R used to predict top 50SNP in gene based association for complex polygenic trait expressed as age of onset of puberty/reproductive fitness in beef cattle.

b) Demonstration utility of phenotype predictor using GA-R predictor for prediction of age of onset of puberty/reproductive fitness with a correlation of 0.72-0.76 to phenotype in heifers which could therefore be measured at birth to be predictive of animals subsequent lifetime performance.

c) The use of MBV in bull and cow selection to improve age of onset of puberty/reproductive fitness in heifers—an example of a sex limited trait for genetic improvement when measured by markers and MBV predictors.

The GA-R module was used to find important SNP responsible for variation in the trait ‘Age at First Corpus Luteum’ in 578 Brahman Heifers. 9775 SNPs were genotyped, and 5363 used in analysis after QC of data.

As the GA is not guaranteed to find a global optimum five analyses were undertaken to identify SNP that were important in all models. The list of the top 50 such SNP were identified and together with results from single SNP analyses and other methods have been used as the basis for gene identification.

The phenotypes for this trait were direct observations on the heifers. After adjustment for systematic non-genetic effects they had a phenotypic standard deviation of 115.2 days. The correlation between MBVs and phenotypes from the five analyses ranged between 0.72-0.76 corresponding to a standard deviation of the MBVs ranging from 82-85 days and a heritability of approximately 0.5.

### REFERENCES

References cited herein are listed on the following pages, and are incorporated herein by this reference:
Gianola, D., R. L. Fernando and A. Stella, 2006: Genomic-assisted prediction of genetic value with semiparametric procedures. Genetics 173: 1761-1776
Bernardo R. and J. Yu, 2007 Prospects for Genomewide Selection for Quantitative Traits Maize. Crop Sci 2007 47: 1082-1090
Bellman, R. (1961). Adaptive control processes: a guided tour. Princeton, N.J.: Princeton University Press. Genetic Analysis Workshop 13: Analysis of Longitudinal Family Data for Complex Diseases and Related Risk Factors: L. Almasy, C. I. Amos, J. E. Bailey-Wilson, R. M. Cantor, C. E. Jaquish, M. Martinez, R. J. Neuman, J. M. Olson, L. J. Palmer, S. S. Rich, M. A. Spence and J. W. MacCluer BMC Genetics 2003, 4(Suppl 1):S1
Efron, B., & Tibshirani, R. J. (1993) An introduction to the bootstrap. Monographs on statistics and applied probability 57 Chapman and Hall, NY
Horne, B. D. and Camp, N. J. (2004). Principal component analysis for selection of optimal SNP-sets that capture intragenic genetic variation. Genetic Epidemiology, 26:11-21.
Johnson, R. A. and Wichern, D. W., editors (1988). Applied multivariate statistical analysis. Prentice-Hall, Inc., Upper Saddle River, N.J., USA.
Lin, Z. and Altman, B. (2004). Finding haplotype tagging SNPs by use of principal components analysis. American Journal of Human Genetics, 75:850-861.
Meuwissen, T. H. E., A. Karlsen, S. Lien, I. Olsaker, and M. E. Goddard (2002) Fine Mapping of a Quantitative Trait Locus for Twinning Rate Using Combined Linkage and Linkage Disequilibrium Mapping Genetics 161, 373-379
Meuwissen, T. H. E., B. J. Hayes, and M. E. Goddard (2001) Prediction of total genetic value using genome-wide dense marker maps Genetics 157 1819-1829
Roweis, S. (1998). EM algorithms for pca and spca. In NIPS '97: Proceedings of the 1997 conference on Advances in neural information processing systems 10, pages 626-632, Cambridge, Mass., USA. MIT Press.
Schaeffer, L. R. (2006). Strategy for applying genome-wide selection in dairy cattle J. Anim. Breed. Genet. 123 218-223
Sharma, S. (1996). Applied multivariate techniques. John Wiley & Sons, Inc., New York, N.Y., USA.
Valdar, W., Solberg, L. C., Gauguier, D., Cookson, W. O., Rawlins, J. N. P., Mott, R., and Flint, J. (2006). Genetic and environmental effects on complex traits in mice. Genetics, 174:959-984
Zabaneh, D. and I. J. Mackay: Genome-wide linkage scan on estimated breeding values for a quantitative trait BMC Genetics 2003, 4(Suppl 1):S61
Zenger et. al (2007) K. R. Zenger, M. S. Khatkar, B. Tier, M. Hobbs, J. A. L. Cavanagh, J. Solkner, R. J. Hawken, W. Barris, H. W. Raadsma Qc analyses of snp array data: experiences from a large population of dairy sires with 23.8 million data points. Association for the Advancement of animal breeding and Genetics (AAABG) Conference paper 17th Annual Conference 23 Sep. 2007

TABLE 12
Listing of Available SNP/Marker Data Sets
(*National Centre for Biotechnology Information U.S. National Library of Medicine 8600 Rockville Pike,
Bethesda, MD 20894 Pubmed Unique Identifier or Web address)

| | | | Unique Identifier or |
|---|---|---|---|
| Species | | Publication or access point | Web address* |
| HUMAN | | | |
| Human | Adverse drug | Bresalier et al., N Engl J Med. 2005 Mar | 15713943 |
| | reaction | 17; 352(11): 1092-102. Epub 2005 Feb 15. | |
| | (example of a | | |
| | trait) | | |
| Human | Alcoholism | Wang et al., BMC Genet. 2005 Dec 30; 6 Suppl 1:S28 | 16451637 |
| Human | Alcoholism | Namkung et al., BMC Genet. 2005 Dec 30; 6 Suppl | 16451705 |
| | | 1:S9 | |
| Human | Alzheimer's | Australian Imaging Biomarkers and Lifestyle (AIBL) | http://www.aibl.nnf.com.au/page/home |
| | | Flagship Study of Ageing; Edith Cohan Univeristy, | |
| | | 184 Hampton Rd Nedland Western Australia; | |
| | | www.aibl.nnf.com.au/page/home; | |
| Human | Alzheimer's - | Coon et al., J Clin Psychiatry. 2007 Apr; 68(4): 613-8. | 17474819 |
| Human | Alzheimer's | Grupe etal., 1: Hum Mol Genet. 2007 Apr | 17317784 |
| | | 15; 16(8): 865-73 | |
| Human | ALS - | Shymick et al., Lancet Neurol. 2007 Apr; 6(4): 322-8 | 17362836 |
| | Amyotrophic | | |
| | lateral | | |
| | sclerosis | | |
| Human | ALS - | Dunckley et al., N Engl J Med. 2007 Aug | 17671248 |
| | Amyotrophic | 23; 357(8): 775-88 | |
| | lateral | | |
| | sclerosis | | |
| Human | Ankylosing | The Wellcome Trust Case Control Consortium | www.wtccc.org.uk/info/overview.shtml |
| | spondylitis | (WTCCC) The Wellcome Trust 215 Euston Road | |
| | | London NW1 2BE | |
| | | Fax: 020 7611 7388; http://www.wtccc.org.uk | |
| Human | Autoimmune | The Wellcome Trust Case Control Consortium | www.wtccc.org.uk/info/overview.shtml |
| | thyroid disease | (WTCCC) The Wellcome Trust 215 Euston Road | |
| | | London NW1 2BE | |
| | | Fax: 020 7611 7388; http://www.wtccc.org.uk | |
| Human | Benign | Lee et al., Hum Mol Genet. 2006 Jan 15; 15(2): 251-8 | 16330481 |
| | recurrent | | |
| | vertigo | | |
| Human | Bipolar | Center for Human Genetic Research MGH Simches | http://www.massgeneral.org/chgr/researchgenes.htm |
| | Disorder | Research Center 185 Cambridge Street Room CPZN | |
| | | 5.821A Boston, MA, 02114 | |
| | | http://www.massgeneral.org/chgr/research_genes.htm | |
| Human | Bipolar\| | Marcheco-Teruel et al., Am J Med Genet B | 16917938 |
| | Disorder | Neuropsychiatr Genet. 2006 Dec 5; 141(8): 833-43 | |
| Human | Bipolar | The Wellcome Trust Case Control Consortium | www.wtccc.org.uk/info/overview.shtml |
| | Disorder | (WTCCC) The Wellcome Trust 215 Euston Road | |
| | | London NW1 2BE | |
| | | Fax: 020 7611 7388; http://www.wtccc.org.uk | |
| Human | Bipolar | Baum et al., Mol Psychiatry. 2007 May 8 | 17486107 |
| | Disorder | | |
| Human | BMI | Lyon et al., PLoS Genet. 2007 Apr 27; 3(4): e61 | 17465681 |
| Human | Cancer - | Hu et al., Cancer Res. 2005 Apr 1; 65(7): 2542-6 | 15805246 |
| | esophogeal | | |
| Human | Cancer - | The Wellcome Trust Case Control Consortium | www.wtccc.org.uk/info/overview.shtml |
| | Breast | (WTCCC) The Wellcome Trust 215 Euston Road | |
| | | London NW1 2BE | |
| | | Fax: 020 7611 7388; http://www.wtccc.org.uk | |
| Human | Cancer - | National Cancer Institute - Cancer Genetic Markers of | http://cgems.cancer.gov/data/ |
| | breast | Susceptibility (CGEMS), 6116 Executive Boulevard | |
| | | Room 3036A, Bethesda, MD 20892-8322 | |
| | | www.cancer.gov & cgems.cancer.gov/data/ | |
| Human | Cancer - | Hunter et al., Nat Genet. 2007 Jul; 39(7): 870-4 | 17529973 |
| | breast | | |
| Human | Cancer - | Easton et al., Nature. 2007 Jun 28; 447(7148): 1087-93 | 17529967 |
| | breast | 93 | |
| Human | Cancer - | Kemp et al., Hum Mol Genet. 2006 Oct | 16923799 |
| | colorectal | 1; 15(19): 2903-10 | |
| Human | Cancer - | Tomlinson et al., Nat Genet. 2007 Aug; 39(8): 984-988 | 17618284 |
| | colorectal | | |
| Human | Cancer - CLL | Sellick et al., Am J Hum Genet. 2005 Sep; 77(3): 420-9 | 16080117 |
| Human | Cancer - CLL | Sellick et al., Blood. 2007 Aug 8 | 17687107 |
| Human | Cancer - Lung | Spinola et al., Cancer Lett. 2007 Jun 28; 251(2): 311-6 | 17223258 |
| Human | Cancer - | Gudmundsson et al., Nat Genet. 2007 | 17401366 |
| | Prostate | May; 39(5): 631-7 | |
| Human | Cancer - | Yeager et al., Nat Genet. 2007 May; 39(5): 645-9 | 17401363 |
| | Prostate | | |
| Human | Cancer - | National Cancer Institute - Cancer Genetic Markers | http://cgems.cancer.gov/data/ |
| | Prostate | of Susceptibility (CGEMS) 6116 Executive | |
| | (CGEMS 1a) | BoulevardRoom 3036A Bethesda, MD 20892-8322 | |
| | | www.cancer.gov & cgems.cancer.gov/data/ | |
| Human | Celiac | van Heel et al., Nat Genet. 2007 Jul; 39(7): 827-9 | 17558408 |
| Human | Chiari type I | Boyles et al., Am J Med Genet A. 2006 Dec | 17103432 |
| | malformation | 15; 140(24): 2776-85 | |
| Human | Coronary Heart | The Wellcome Trust Case Control Consortium | www.wtccc.org.uk/info/overview.shtml |
| | Disease | (WTCCC) The Wellcome Trust 215 Euston Road | |
| | | London NW1 2BE | |
| | | Fax: 020 7611 7388; http://www.wtccc.org.uk | |
| Human | Crohns | Libioulle et al., PLoS Genet. 2007 Apr 20; 3(4): e58 | 17447842 |
| | disease - | | |
| Human | Crohns | Hampe et al., Nat Genet. 2007 Feb; 39(2): 207-11. | 17200669 |
| | disease - | Epub 2006 Dec 31 | |
| Human | Crohns | Rioux et al., Nat Genet. 2007 May; 39(5): 596-604 | 17435756 |
| | disease - | | |
| Human | Crohn's | The Wellcome Trust Case Control Consortium | www.wtccc.org.uk/info/overview.shtml |
| | Disease | (WTCCC) The Wellcome Trust 215 Euston Road | |
| | | London NW1 2BE | |
| | | Fax: 020 7611 7388; http://www.wtccc.org.uk | |
| Human | Cleft lip/Cleft | Riley et al., Am J Med Genet A. 2007 Apr | 17366557 |
| | Palate | 15; 143(8): 846-52 | |
| Human | Diabetes - type 1 | The Wellcome Trust Case Control Consortium | www.wtccc.org.uk/info/overview.shtml |
| | | (WTCCC) The Wellcome Trust 215 Euston Road | |
| | | London NW1 2BE | |
| | | Fax: 020 7611 7388; http://www.wtccc.org.uk | |
| Human | Diabetes - type 1 | Smyth et al., Nat Genet. 2006 Jun; 38(6): 617-9 | 16699517 |
| Human | Diabetes - type 2 | Diabetes Genetics Initiative of Broad Institute of | 17463246 |
| | | Harvard and MIT et al., Science. 2007 Jun | |
| | | 1; 316(5829): 1331-6 | |
| Human | Diabetes - type 2 | Sladek et al., Nature. 2007 Feb 22; 445(7130): 881-5 | 17293876 |
| Human | Diabetes - type 2 | The Wellcome Trust Case Control Consortium | www.wtccc.org.uk/info/overview.shtml |
| | | (WTCCC) The Wellcome Trust 215 Euston Road | |
| | | London NW1 2BE | |
| | | Fax: 020 7611 7388; http://www.wtccc.org.uk | |
| Human | Diabetes - type 2 | Zeggini et al., Science. 2007 Jun 1; 316(5829): 1336-41 | 17463249 |
| Human | Diabetes - type 2 | Scott et al., Science. 2007 Jun 1; 316(5829): 1341-5 | 17463248 |
| Human | Diabetes - type 2 | Maeda, Diabetes Res Clin Pract. 2004 Dec; 66 Suppl | 15563979 |
| | Complications - | 1:S45-7 | |
| | Nephropathy | | |
| Human | Diabetes - type 2 | Maeda et al., Kidney Int Suppl. 2007 Aug; (106): S43-8 | 17653210 |
| | Complications - | | |
| | Nephropathy | | |
| Human | Diabetes - type 2 | Tanaka et al., Diabetes. 2003 Nov; 52(11): 2848-53 | 14578305 |
| | Complications - | | |
| Human | Diabetes - | Looker et al., Diabetes. 2007 Apr; 56(4): 1160-6 | 17395753 |
| | Complications - | | |
| | Retinopathy | | |
| Human | Framingham | Herbert et al., Nat Genet. 2007 Feb; 39(2): 135-6 | 17262019 |
| | Heart | | |
| Human | Gallstone | Buch et al., Nat Genet. 2007 Aug; 39(8): 995-999 | 17632509 |
| | Disease | | |
| Human | Hypertension - | Bella et al., Hypertension. 2007 Mar; 49(3): 453-60 | 17224468 |
| Human | Hypertension | The Wellcome Trust Case Control Consortium | www.wtccc.org.uk/info/overview.shtml |
| | | (WTCCC) The Wellcome Trust 215 Euston Road | |
| | | London NW1 2BE | |
| | | Fax: 020 7611 7388; http://www.wtccc.org.uk | |
| Human | Ischaemic | Matarin et al., Lancet Neurol. 2007 May; 6(5): 414-20 | 17434096 |
| | Stroke | | |
| Human | Mental | Hoyer et al., J Med Genet. 2007 Jun 29 | 17601928 |
| | Retardation | | |
| Human | Multiple | Sawcer et al., Am J Hum Genet. 2005 Sep; 77(3): 454-67 | 16080120 |
| | sclerosis | | |
| Human | Multiple | The Wellcome Trust Case Control Consortium | www.wtccc.org.uk/info/overview.shtml |
| | Sclerosis | (WTCCC) The Wellcome Trust 215 Euston Road | |
| | | London NW1 2BE | |
| | | Fax: 020 7611 7388; http://www.wtccc.org.uk | |
| Human | Myocardial | Ozaki and Tanaka, Cell Mol Life Sci. 2005 | 15990958 |
| | Infarction | Aug; 62(16): 1804-13 | |
| Human | Nicotine | Bierut et al., Hum Mol Genet. 2007 Jan 1; 16(1): 24-35 | 17158188 |
| | dependence | | |
| Human | Nicotine | Uhl et al., BMC Genet. 2007 Apr 3; 8:10 | 17407593 |
| | dependence | | |
| Human | Obesity-related | Scuteri et al., PLoS Genet. 2007 Jul 20; 3(7): e115 | 17658951 |
| | traits | | |
| Human | Obesity | NGFN Project Management, Projektträger im DLR | www.science.ngfn.de/6_178.htm |
| | | Heinrich-Konen-Straβe 1 53227 Bonn at Universität | |
| | | zu Koln Zülpicher Str 47 50674 Köln | |
| | | http://www.science.ngfn.de/6_178.htm | |
| Human | Obesity (Lyon) | Lyon et al., PLoS Genet. 2007 Apr 27; 3(4): e61 | 17465681 |
| | | Duplication | |
| Human | Olfactory | Knaapila et al., Eur J Hum Genet. 2007 | 17342154 |
| | sense - | May; 15(5): 596-602 | |
| | Identification; | | |
| | Intensity; | | |
| | pleasantness | | |
| Human | Osteoarthritis | Abel et al., Autoimmun Rev. 2006 Apr; 5(4): 258-63 | 16697966 |
| Human | Parkinsons | Fung et al., Lancet Neurol 2006; 5: 911-916 | |
| | Disease | | |
| Human | Rheumatoid | The Wellcome Trust Case Control Consortium | www.wtccc.org.uk/info/overview.shtml |
| | Arthritis | (WTCCC) The Wellcome Trust 215 Euston Road | |
| | (Wellcome | London NW1 2BE | |
| | Trust) | Fax: 020 7611 7388; http://www.wtccc.org.uk | |
| Human | Rheumatoid | Amos et al., Genes Immun. 2006 Jun; 7(4): 277-86 | 16691188 |
| | Arthritis | | |
| Human | Rheumatoid | John et al., Am J Hum Genet. 2004 Jul; 75(1): 54-64 | 15154113 |
| | Arthritis | | |
| Human | Rheumatoid | Tamiya et al., Hum Mol Genet. 2005 Aug | 16000323 |
| | Arthritis | 15; 14(16): 2305-21 | |
| Human | Sarcoidosis | Institute of Human Genetics, University of Lübeck, | http://www.science.ngfn.de/dateien/ |
| | | Ratzeburger Allee 160, 23538 Lübeck, Germany | NUW-S26T11_Schuermann.pdf |
| Human | Situs Defect | Gutierrez-Roelens et al. Eur J Hum Genet. 2006 | 16639409 |
| | (Gutierrez) | Jul; 14(7): 809-15 | |
| Human | Tuberculosis | The Wellcome Trust Case Control Consortium | www.wtccc.org.uk/info/overview.shtml |
| | | (WTCCC) The Wellcome Trust 215 Euston Road | |
| | | London NW1 2BE | |
| | | Fax: 020 7611 7388; http://www.wtccc.org.uk | |
| Human | Malaria | The Wellcome Trust Case Control Consortium | www.wtccc.org.uk/info/overview.shtml |
| | | (WTCCC) The Wellcome Trust 215 Euston Road | |
| | | London NW1 2BE | |
| | | Fax: 020 7611 7388; http://www.wtccc.org.uk | |
| BOVINE | | | |
| Bovine | Example of | National Animal Genome Research Program - | http://www.animalgenome.org/cattle/ |
| | markers | Cattle Genome; Texas A&M Univeristy | |
| Dairy | Example of | Australin Dairy Herd Improvement Scheme | http://www.australiandairyfarmers.com.au/ |
| | traits | Australian Dairy Farmers Limited, Level 6 | |
| | | 84 William Street Melbourne VIC 3000 | |
| Beef | Example of | BREEDPLAN at University of New England | http://breedplan.une.edu.au/ |
| | traits | Armidale, NSW 2351 AUSTRALIA | |
| MOUSE | | | |
| Mouse | For access to | Wellcome Trust Center for Human Genetics The | http://gscan.well.ox.ac.uk/#phenotyes |
| | markers and | Genetic Architecture of Complex Traits in | |
| | traits | Heterogeneous Stock Mice Roosevelt Drive Oxford, | |
| | | OX3 7BN, United Kingdom, | |
| | | http://gscan.well.ox.ac.uk/#phenotypes | |
| DOG | | | |
| Dog | Example of | Dog Genome Broad Institute 7 Cambridge Center | http://www.broad.mit.edu/mammals/dog/ |
| | markers | Cambridge, MA 02142 USA | |
| | | http://www.broad.mit.edu/mammals/dog/ | |
| Dog | For access to | Agrafioti and Stumpf, Nucleic Acids Res. 2007 | 17202172 |
| | markers | Jan; 35(Database issue): D71-5 | |
| Dog | For markers | Leegwater et al., J Hered. 2007 Aug 3 | 17548862 |
| | and traits | | |
| Dog | Example of | Lindblad-Toh et al., Nature. 2005 Dec | 16341006 |
| | markers | 8; 438(7069): 803-19 | |
| Dog | For access to | Lindblad-Toh, K. A. W101 Trait Mapping Using A | http://www.intl-pag.org/15/abstracts/ |
| | markers and | Canine SNP Array: A Model For Equine Genetics. | PAG15_W17_101.html |
| | traits | Plant & Animal Genomes XV Conference January | |
| | | 13-17, 2007 Town & Country Convention Center San | |
| | | Diego, CA; http://www.intl-pag.org/15/abstracts/ | |
| | | PAG15_W17_101.html | |
| HORSE | | | |
| Horse | Example of | Agrafioti and Stumpf, Nucleic Acids Res. 2007 | 17202172 |
| | markers | Jan; 35(Database issue): D71-5 Duplication | |
| Horse | Example of | Horse Genome Project; Cornell University - College of | http://web.vet.cornell.edu/ |
| | markers | Veterinary Medicine Ithaca, New York 14853-6401 | public/research/zweig/antczak07.htm |
| Horse | Example of | Horse Genome MIT Broad Institute 7 Cambridge | http://www.broad.mit.edu/mammals/horse/snp/ |
| | markers | Center | |
| | | Cambridge, MA 02142 USA | |
| | | http://www.broad.mit.edu/mammals/horse/ | |
| Horse | Example of | National Animal Genome Research Program - | http://www.uky.edu/Ag/Horsemap/ |
| | markers | Horse Genome; Univeristy of Kentucky | |
| Horse | Example of a | Dranchak PK,, J Am Vet Med Assoc. 2005 Sep | 16178398 |
| | trait | 1; 227(5): 762-7. | |
| Horse | Example of a | Perryman LE, Torbeck RL. J Am Vet Med Assoc. | 7429919 |
| | trait | 1980 Jun 1; 176(11): 1250-1. | |
| Horse | Example of | Mark Read's Ozeform supported by Read Interactive | http://www.ozeform.com/ |
| | traits | | |
| Horse | Example of a | New Zealand's Thoroughbreed Breeder's Association | http://www.nzthoroughbred.co.nz/Contact-Us.aspx |
| | trait | Gate 8, Derby Enclosure, Ellerslie Racecourse | |
| | | Morrin Street, Ellerslie, AUCKLAND | |
| Horse | Example of | Expert Form.com 259A Keilor Rd | http://www.expertform.com/ |
| | traits | Essendon 3040 Vic | |
| Horse | Example of | Timeform, 25 Timeform House Northgate | http://www.timeform.co.uk/ |
| | traits | Halifax | |
| | | HX1 1XF | |
| SHEEP | | | |
| Sheep | Example of | International Sheep Genomics Consortium | http://www.sheephapmap.org/isgc_snpchip.htm |
| | markers | http://www.sheephapmap.org/ Secretary c/o CSIRO | |
| | | Livestock Industries Queensland Bioscience Precinct - | |
| | | St Lucia Queensland Bioscience Precinct 306 | |
| | | Carmody Road St Lucia QLD 4067 Australia | |
| Sheep | Example of | National Animal Genome Research Program Sheep | http://www.animalgenome.org/sheep/ |
| | markers | Genome; Utah State University | |
| | Example of a | Raadsma et al., Rev Sci Tech. 1998 Apr; 17(1): 315-28. | 9638820 |
| | trait | Review. | |
| | Examples of | Sheep Genetics Australia at University of New | http://www.sheepgenetics.org.au/ |
| | traits | England | |
| | | Armidale, NSW 2351 AUSTRALIA | |
| PIG | | | |
| Pig | Example of | National Animal Genome Research Program - | http://www.animalgenome.org/pigs/ |
| | markers | Pig Genome; Iowa State University | |
| | | http://www.animalgenome.org/pigs/ | |
| Pig | Example of | Panitz et al., Bioinformatics. 2007 Jul 1; 23(13): i387-91 | 17646321 |
| | markers | | |
| Pig | Example of | Chen et al., Int J Biol Sci. 2007 Feb 10; 3(3): 153-65. | 17384734 |
| | markers | | |
| | Example of a | Schneider et al., Anim Reprod Sci. 1998 Feb | 9615181 |
| | trait | 27; 50(1-2): 69-80. | |
| Pig | Example of | PIGBLUP at University of New England | http://agbu.une.edu.au/pigs/pigblup/index1.php |
| | traits | Armidale, NSW 2351 AUSTRALIA | |
| CHICKEN | | | |
| Chicken | Example of | National Animal Genome Research Program - | http://poultry.mph.msu.edu/ |
| | markers | Chicken Genome; Michigan State Univeristy | |
| Chicken | Example of a | Ye, X. et al., Poult Sci. 2006 Sep; 85(9): 1555-69 | 16977841 |
| | trait | | |
| Aquaculture | | Z. J. Liu, and J. F. Cordesb., Aquaculture Volume | |
| | | 238, Issues 1-4, 1 Sep. 2004, Pages 1-37 | |
| OYSTERS | | | |
| Oysters | Example of a | Evans, S., et al 2004. Aquaculture 230: 89-98. | |
| | trait | | |
| Oysters | Example of | Quilang et al., BMC Genomics. 2007 Jun 8; 8: 157 | 17559679 |
| | markers | | |
| Oysters | Example of | NAGRP Aquaculture Genome Projects | http://www.animalgenome.org/aquaculture/oysters/ |
| | markers | College of Marine Studies, University of Delaware | |
| | | 700 Pilottown Road, Lewes, DE 19958 | |
| SALMONIDS | | | |
| salmon | Example of | Salmon Genome Project Address c/- Department of | http://www.salmongenome.no/cgi-bin/sgp.cgi |
| | markers | Informatics and Computational Biology Unit, Bergen | |
| | | Centre for Computational Science University of | |
| | | Bergen | |
| | | HIB N5020 BERGEN NORWAY | |
| salmon | Example of | Anderson et al., Genetics. 2006 Apr; 172(4): 2567-82. | 16387880 |
| | markers | Epub 2005 Dec 30 | |
| salmon | Example of | Hayes BJ, et al Heredity. 2006 Jul; 97(1): 19-26. Epub | 16685283 |
| | markers and | 2006 May 10 | |
| | traits | | |
| salmon | Example of a | Moghadam HK, Mol Genet Genomics. 2007 | 17308931 |
| | trait | Jun; 277(6): 647-61. Epub 2007 Feb 17 | |
| | Example of | The USDA/ARS National Center for Cool and Cold | http://www.animalgenome.org/ |
| | markers and | Water Aquaculture 11861 Leetown Road | aquaculture/salmonids/genetmrker.html |
| | traits | Kearneysville, West Virginia 25430 Phone 304-724- | |
| | | 8340x2129 | |
| Trout | Example of | Smith et al., Mol Ecol. 2005 Nov; 14(13): 4193-203 | 16262869 |
| | markers | | |
| Trout | Example of a | Moghadam HK, Mol Genet Genomics. 2007 | 17308931 |
| | trait | Jun; 277(6): 647-61. Epub 2007 Feb 17 | |
| SHRIMP | | | |
| shrimp | Example of | NAGRP Aquaculture Genome Projects - Department | http://www.animalgenome.org/aquaculture/shrimp/ |
| | markers | of Biochemistry Medical University of South Carolina | |
| | | A204 Hollings Marine Laboratory 331 Fort Johnson | |
| | | Road | |
| | | Charleston, SC 29412 | |
| shrimp | Example of | Black Tiger Shrimp EST project - Shrimp Molecular | http://pmonodon.biotec.or.th/background.html |
| | markers | Biology and Genomic Research Laboratory, | |
| | | Department of Biochemistry, Faculty of Science, | |
| | | Chulalongkorn University, Bangkok 10330 | |
| shrimp | Example of a | Arcos, TG., - Aquaculture Volume 236, Issues 1-4, 14 | http://www.sciencedirect.com/science?_ob= |
| | trait | Jun. 2004, Pages 151-165 | ArticleURL&_udi=B6T4D- |
| | | | 4C7DDPT-1&_user=10&_coverDate= |
| | | | 06%2F14%2F2004&_rdoc= |
| | | | 1&_fmt=&_orig= |
| | | | search&_sort=d&view=c&_acct= |
| | | | C000050221&_version= |
| | | | 1&_urlVersion=0&_userid= |
| | | | 10&md5=868920ccc407ba4205d6838d8bdcc972 |
| PLANTS/CROPS | | | |
| ARABIDOPSIS | | | |
| Arabidopsis | Example of | Kim et al., Nat Genet. 2007 Aug 5 | 17676040 |
| thaliana | markers | | |
| Arabidopsis | Example of a | Kearsey MJ et al., Heredity November 2003, Volume | 14576738 |
| thaliana | trait | 91, Number 5, Pages 456-464 | |
| BARLEY | | | |
| Barley | Example of | Rostoks et al., Mol Genet Genomics. 2005 | 16244872 |
| | markers | Dec; 274(5): 515-27 | |
| Barley | Example of a | Hori et al Theor Appl Genet. 2007 Aug 22; | 17712544 |
| | trait | | |
| WHEAT | | | |
| Wheat | Example of | International wheat genome sequencing project | http://www.wheatgenome.org/contact.html |
| | markes | c/- Eversole Associates, 5207 Wyoming Road | |
| | | Bethesda, MD 20816 USA | |
| Wheat | Example of | Wheat SNP database - University of California, | http://wheat.pw.usda.gov/SNP/new/index.shtml |
| | markes | Davis Dept. of Plant Sciences, University of | |
| | | California, One Shields Avenue, Davis, CA 95616 | |
| Wheat | Example of a | Kuchel H, etal., Theor Appl Genet. 2007 Aug 23 | 17713755 |
| | trait | | |
| Wheat | Example of a | Marza F., et al Theoretical and Applied Genetics | http://www.springerlink.com/ |
| | trait | Volume 19, Number 2/February, 2007 163-177 | content/y025362072847608/ |
| RICE | | | |
| Rice | Example of | Zhang et al., DNA Res. 2007 Feb 28; 14(1): 37-45 | 17452422 |
| | markers | | |
| Rice | Example of | Feltus et al., Genome Res. 2004 Sep; 14(9): 1812-9 | 15342564 |
| | markers | | |
| Rice | Example of | Plant Physiol. 2004 Jul; 135(3): 1198-205. | 15266053 |
| | markers | | |
| Rice | Example of | Liu, CG et al., Yi Chuan. 2006 Jun; 28(6): 737-44. | 16818440 |
| | markers | | |
| Rice | Example of a | Cho et al., Mol Cells. 2007 Feb 28; 23(1): 72-9 | 17464214 |
| | trait | | |
| Rice | Example of a | Lian X et al., Theor Appl Genet. 2005 Dec; 112(1): 85-96. | 16189659 |
| | trait | Epub 2005 Sep 28 | |
| PINE | | | |
| Pine | Example of | Tree Genes - A forest tree genome database | http://dendrome.ucdavis.edu/treegenes/ |
| | markers | University of California, Davis Dept. of Plant | |
| | | Sciences, University of | |
| | | California, One Shields Avenue, Davis, CA 95616 | |
| Pine | Example of | The Pine Genome Initiative c/- The institute of Forest | http://pinegenomeinitiative.org/deliver.html |
| | markers | Biotechnology 920 Main Campus Drive, Suite 101 | |
| | | Raleigh, NC 27606 | |
| Pine | Example of a | Brown GR, et al Genetics. 2003 Aug; 164(4): 1537-46 | 12930758 |
| | trait | | |
| Pine | Example of a | Southern Tree Breeding Association, 2 Eleanor | http://www.stba.com.au/treeplan.html |
| | trait | Street | |
| | | PO Box 1811 Mount Gambier, SA 5290 Australia | |

1. A method for the prediction of the merit of at least one individual in a population, the method comprising the steps of:
(a) in the population, where information of individuals are known, using dimension reduction on the information to project the information to a low dimensional space whilst retaining the complexity of the information to generate a set of explanatory variables;
(b) utilising the explanatory variables to generate a predictor function with respect to merit; and
(c) utilising the predictor function to predict the merit of the individual.

2. A method as claimed in claim 1 for a prediction of a merit of at least one individual, the method comprising the steps of:
(a) in a first population, where genotype and phenotype information of individuals in the first population are known, using dimension reduction on the genotype and phenotype information to determine the complexity of the genotype and phenotype information to minimise prediction error for at least one marker in the first population and thereby generate a set of explanatory variables with respect to the at least one marker;
(b) utilising the explanatory variables to the first population to generate a predictor function with respect to merit;
(c) generating a genotype for the at least one marker in at least one individual of interest from a second population; and
(d) utilising the predictor function to the genotype of the at least one individual of interest to determine the genetic merit of the individual of interest with respect to the at least one marker.

3. A method for the prediction of the merit of at least one individual in a population, the method comprising the steps of:
(a) in the population, where information of individuals are known, using a genetic algorithm process on the information to generate a set of explanatory variables for all the information, the explanatory variables comprising weighted averages for components of the information; and
(b) utilising the explanatory variables to generate a predictor function with respect to merit;
(c) utilising the predictor function to predict the merit of the individual.

4. A method as claimed in claim 1 wherein step (b) comprises utilising the explanatory variables to generate a plurality of predictor functions for the individuals of the population.

5. A method as claimed in claim 1 wherein the information comprises information for at least one marker.

6. A method as claimed in claim 5 wherein the information comprises information for a plurality of marker s.

7. A method as claimed in claim 1 wherein for a plurality of individuals of interest from the population where information is unknown, generating genotype for at least one individual of interest from population.

8. A method according to claim 1 further comprising the steps of:
(f) determining additional information on the explanatory variables for the at least one individual;
(g) combining the additional information for the at least one individual with the information on the explanatory variables for the individuals of the population; and
(h) repeating steps (b) and (c) for at least one further individual to predict the merit of the further individual.

9. A method according to claim 8 wherein step (f) comprises determining additional information on the explanatory variables on a plurality of individuals.

10. A method according to claim 1, wherein the utilisation of the predictor function is performed on the basis of a desired outcome.

11. A method according to claim 4 wherein the genotype information comprises genetic markers or bio-markers or epigenetic markers.

12. A method according to claim 1, wherein the merit is a genetic merit selected from the group of a molecular breeding value, a quantitative trait locus, or a quantitative trait nucleotide.

13. A method of predicting trait performance for at least one individual in a population, the method comprising the steps of:
(a) in the population, where information of individuals are known, using dimension reduction on the information to project the information to a low dimensional space whilst retaining the complexity of the information to generate a set of explanatory variables; and
(b) utilising the explanatory variables to generate a predictor function with respect to merit;
(c) utilising the predictor function to predict the trait performance for the individual.

14. A method as claimed in claim 13 further comprising the steps of:
(d) for an individual of interest from the population where information is unknown, generating genotype for at least one individual of interest from population; and
(e) applying the predictor function to the genotype of the at least one individual of interest to predict the predict the trait performance for the individual.

15. A method as claimed in claim 13 wherein the information is selected from the group of genotype, phenotype or genotype and phenotype information on individuals in the population.

16. A method as claimed in claim 13 wherein the trait is a quantitative trait.

17. A method for selecting at least one individual in a population, the method comprising the steps of:
(a) in the population, where information of individuals are known, using dimension reduction on the information to project the information to a low dimensional space whilst retaining the complexity of the information to generate a set of explanatory variables; and
(b) utilising the explanatory variables to generate a predictor function;
(c) utilising the predictor function to select an individual.

18. A method as claimed in claim 17 further comprising the steps of:
(d) for an individual of interest from the population where information is unknown, generating genotype for at least one individual of interest from population; and
(e) applying the predictor function to the genotype of the at least one individual of interest to select an individual.

19. A method as claimed in claim 17 wherein the information is selected from the group of genotype, phenotype or genotype and phenotype information on individuals in the population.

20. A method of diagnosing a condition in at least one individual of interest in a population, the method comprising the steps of:
(a) in the population, where information of individuals are known, using dimension reduction on the information to project the information to a low dimensional space whilst retaining the complexity of the information to generate a set of explanatory variables; and
(b) utilising the explanatory variables to generate a predictor function;
(c) utilising the predictor function to diagnose a condition in the individual.

21. A method as claimed in claim 20 further comprising the steps of:
(d) for an individual of interest from the population where information is unknown, generating genotype for at least one individual of interest from population; and
(e) applying the predictor function to the genotype of the at least one individual of interest to diagnose a condition in the individual of interest.

22. A method as claimed in claim 20 wherein the information is selected from the group of genotype, phenotype or genotype and phenotype information on individuals in the population.

23. A method of prediction of a susceptibility to an outcome of at least one individual of interest in a population, the method comprising the steps of:
(a) in the population, where information of individuals are known, using dimension reduction on the information to project the information to a low dimensional space whilst retaining the complexity of the information to generate a set of explanatory variables; and
(b) utilising the explanatory variables to generate a predictor function;
(c) utilising the predictor function to predict the susceptibility of the individual to an outcome.

24. A method as claimed in claim 23 further comprising the steps of:
(d) for an individual of interest from the population where information is unknown, generating genotype for at least one individual of interest from population; and
(e) applying the predictor function to the genotype of the at least one individual of interest to predict the susceptibility of the individual to an outcome.

25. A method as claimed in claim 23 wherein the information is selected from the group of genotype, phenotype or genotype and phenotype information on individuals in the population.

26. A method as claimed in claim 23 wherein the outcome is the susceptibility of the individual of interest to a disease.

27. A method as claimed in claim 23 wherein the outcome is the susceptibility of the individual of interest to a response to a stimulus.

28. A method as claimed in claim 27 wherein the stimulus is selected from the group of a medicament, toxin, or an environmental condition.

29. A method as claimed in claim 28 wherein the environmental condition comprises water shortage, feed shortage, stress, sunlight, or other environmental condition.

30. A method of breeding at least one individual in a population, the method comprising the steps of:
(a) in the population, where information of individuals are known, using dimension reduction on the information to project the information to a low dimensional space whilst retaining the complexity of the information to generate a set of explanatory variables; and
(b) utilising the explanatory variables to generate a predictor function with respect to merit of the individual;
(c) utilising the predictor function to predict the merit of the individual and
(d) breeding from the individual of interest on the basis of the merit of the individual.

31. A method according to claim 30, further comprising the steps of:
(f) determining information for the descendants of the at least one individual;
(g) correlating the information for the descendants of the at least one individual to the predictor function; and
(h) selecting descendants of said individual on the basis of the relationship between the information for the descendants and the predictor function.

32. A method as claimed in claim 30 wherein the information is selected from the group of genotype, phenotype or genotype and phenotype information on individuals in the population.

33. A system for the prediction of merit of an individual in a population, the system comprising:
(a) in the population, where information of individuals are known, means for using dimension reduction on the information to project the information to a low dimensional space whilst retaining the complexity of the information to generate a set of explanatory variables; and
(b) means for utilising the explanatory variables to generate a predictor function with respect to merit;
(c) means for utilising the predictor function to predict the merit of the individual.

34. A system for predicting trait performance of at least one individual in a population, the system comprising;
(a) in the population, where information of individuals are known, means for using dimension reduction on the information to project the information to a low dimensional space whilst retaining the complexity of the information to generate a set of explanatory variables; and
(b) means for utilising the explanatory variables to generate a predictor function; and
(c) means for utilising the predictor function to predict performance of said trait for the individual of interest.

35. A system as claimed in claim 34 wherein the trait is a quantitative trait.

36. A system for selecting at least one individual in a population, the system comprising;
(a) in the population, where information of individuals are known, means for using dimension reduction on the information to project the information to a low dimensional space whilst retaining the complexity of the information to generate a set of explanatory variables; and
(b) means for utilising the explanatory variables to generate a predictor function; and
(c) means for utilising the predictor function to select the individual.

37. A system for diagnosing a condition in at least one individual of interest in a population, the system comprising:
(a) in the population, where information of individuals are known, means for using dimension reduction on the information to project the information to a low dimensional space whilst retaining the complexity of the information to generate a set of explanatory variables; and
(b) means for utilising the explanatory variables to generate a predictor function;
(c) means for utilising the predictor function to diagnose a condition in the individual.

38. A system for prediction of a susceptibility to an outcome of at least one individual of interest in a population, the system comprising:
(a) in the population, where information of individuals are known, means for using dimension reduction on the information to project the information to a low dimensional space whilst retaining the complexity of the information to generate a set of explanatory variables; and
(b) means for utilising the explanatory variables to generate a predictor function;
(c) means for utilising the predictor function to predict the susceptibility of the at least one individual of interest to an outcome.

39. A system for breeding at least one individual in a population, the system comprising:
(a) in the population, where information of individuals are known, means for using dimension reduction on the information to project the information to a low dimensional space whilst retaining the complexity of the information to generate a set of explanatory variables; and
(b) means for utilising the explanatory variables to generate a predictor function with respect to merit of the individual;
(c) means for utilising the predictor function to predict the merit of the individual and
(d) means for breeding from the individual of interest on the basis of the merit of the individual.

40. A system as claimed in claim 39, further comprising the steps of:
(f) means for determining information for the descendants of the at least one individual;
(g) means for correlating the information for the descendants of the at least one individual to the predictor function; and
(h) means for selecting descendants of said individual on the basis of the relationship between the information for the descendants and the predictor function.

41. A method according to claim 1, wherein the information comprises genetic information consisting essentially of marker genotypes.

42. A method according to claim 41 wherein the genetic markers are distributed substantially across the genome.

43. A method according to claim 41, wherein the number of genetic markers genotyped is greater than 1000, greater than 1500, greater than 2500, greater than 5000, greater than 10000, greater than 15000, greater than 20000, greater than 25000, greater than 30000, greater than 35000, greater than 40000, greater than 45000, greater than 50000, greater than 100000, greater than 250000, greater than 500000, or greater than 1000000, greater than 5000000, greater than 10000000 or greater than 15000000.

44. A method according to claim 41, wherein the genetic markers are selected from the group consisting of single nucleotide polymorphism (SNP), tag SNP, microsatellite (simple tandem repeat STR, simple sequence repeat SSR), restriction fragment length polymorphism (RFLP), amplified fragment length polymorphism (AFLP), insertion-deletion polymorphism (INDEL), random amplified polymorphic DNA (RAPD), ligase chain reaction, insertion/deletions and direct sequencing of the gene or a simple sequence conformation polymorphisms (SSCP).

45. A method according to claim 44 wherein the genetic marker is a SNP.

46. A method according to claim 1, wherein the information comprises at least one of the pedigree of the individual; an estimated breeding value of the individual; data on genetic markers across the genome for the individual or for relatives of the individual; at least one index of phenotype for the individual or for relatives of the individual; at least one marker predictive of phenotype for the individual or for relatives of the individual; and at least one index of epigenetic modification or status for the individual, or a combination thereof.

47. A method according to claim 13, wherein the individual is a dairy cow or bull, and wherein the quantitative trait is selected from the group consisting of APR, ASI, protein kg, protein percent, milk yield, fat kg, fat percent, overall type, mammary system, stature, udder texture, bone quality, angularity, muzzle width, body depth, chest width, pin set, pin sign, foot angle, set sign, rear leg view, udder depth, fore attachment, rear attachment height, rear attachment width, centre ligament, teat placement, teat length, loin strength, milking speed, temperament, like-ability, survival, calving ease, somatic cell count, cow fertility, and gestation length, or a combination of one or more of these traits.

48. A method according to claim 1, wherein the dimension reduction is selected from the a technique in the group consisting of principal component analysis (PCA), a genetic algorithm, a neural network, partial least squares (PLS), inverse least squares, kernel PCA, LLE, Hessian LLE, Laplacian Eigenmaps, LTSA, isomap, maximum variance unfolding, Bolzman machines, projection pursuit, a hidden Markov model support vector machines, kernel regression, discriminant analysis and classification, k-nearest-neighbour analysis, fuzzy neural networks, Bayesian networks, or cluster analysis.

49. A method according to claim 48, wherein the dimension reduction technique is principal component analysis.

50. A method according to claim 48, wherein the dimension reduction technique is supervised principal component analysis.

51. A method according to claim 49 wherein the number of principal components is between about 10 and about 40.

52. A method according to claim 49 wherein the number of principal components is about 20.

53. A method according to claim 48 wherein the dimension reduction technique is partial least squares analysis.

54. A method according to claim 53 wherein the number of latent components is between about 4 and about 10.

55. A method according to claim 43 wherein the number of latent components is about 6.

56. A method according to claim 48 wherein the dimension reduction technique is support vector machine analysis.

57. A method according to claim 1 wherein the information does not include the pedigree of the individual.

58. A breeders product comprising at least one gamete with a high prediction of merit for at least one marker, the breeders product selected by a method for the prediction of the merit of at least one individual, the method comprising the steps of:
(a) in a first population, where genotype and phenotype information of individuals in the first population are known, using dimension reduction on the genotype and phenotype information to determine the complexity of the genotype and phenotype information to minimise prediction error for at least one marker in the first population and thereby generate a set of explanatory variables with respect to the at least one marker;
(b) applying the explanatory variables to the first population to generate a predictor function;
(c) generating genotype for the at least one marker in at least one individual of interest from a second population;
(d) applying the predictor function to the genotype of the at least one individual of interest to determine the genetic merit of the individual of interest with respect to the at least one marker.

59. A computer system comprising a computer processor and memory, the memory comprising software code stored therein for execution by the computer processor of a method for the prediction of the merit of at least one individual in a population, the method comprising the steps of:
(a) in a database comprising information about the population, where information of individuals are known, using dimension reduction on the information to project the information to a low dimensional space whilst retaining the complexity of the information to generate a set of explanatory variables;
(b) utilising the explanatory variables to generate a predictor function with respect to merit; and
(c) utilising the predictor function to predict the merit of the individual.

60. A computer readable medium, having a program recorded thereon, where the program is configured to make a computer execute a procedure for the prediction of the merit of at least one individual in a population, the software product comprising:
(a) in a database comprising information about the population, where information of individuals are known, code for using dimension reduction on the information to project the information to a low dimensional space whilst retaining the complexity of the information to generate a set of explanatory variables;
(b) code for utilising the explanatory variables to generate a predictor function with respect to merit; and
(c) code for utilising the predictor function to predict the merit of the individual.

61. An information database product comprising information for individuals of a population, the information database for use with a method for the selection of at least one individual in the population, the method comprising the steps of:
(a) in the population, where information of individuals are known, using dimension reduction on the information to project the information to a low dimensional space whilst retaining the complexity of the information to generate a set of explanatory variables; and
(b) utilising the explanatory variables to generate a predictor function with respect to merit;
(c) utilising the predictor function to predict the merit of the individual.

62. An information database product for use with a breeding program, the database comprising information for individuals of a population and a prediction of the merit of the individuals in the population.

63. An information database product comprising information for individuals of a population according to claim 62 wherein a prediction of a merit of the individuals in the population is provided by a dimension reduction method on the genotype and phenotype information of individuals in the population comprising the steps of:
(a) using a dimension reduction method, determining the complexity of genotype and phenotype information of individuals in the population to minimise prediction error and thereby generate a set of explanatory variables;
(b) applying the explanatory variables to the first population to generate a predictor function;
(c) generating genotype for the at least one marker in at least one individual of interest from a second population;
(d) applying the predictor function to the genotype of the individuals of the second population thereby to determine the genetic merit of individuals in the second population individuals with respect to the at least one marker.

64. An information database product according to claim 62 wherein individuals of interest from the second population are selected for use in a breeding program based upon the prediction of merit for the at least one marker.

65. A method as claimed in claim 1 wherein the predictor function is a predictor function with having minimal prediction error.

66. A system according to claim 33 wherein the information comprises genetic information consisting essentially of marker genotypes.

67. A system according to claim 33 wherein the genetic markers are distributed substantially across the genome.

68. A system according to claim 33 wherein the dimension reduction is selected from the a technique in the group consisting of principal component analysis (PCA), a genetic algorithm, a neural network, partial least squares (PLS), inverse least squares, kernel PCA, LLE, Hessian LLE, Laplacian Eigenmaps, LTSA, isomap, maximum variance unfolding, Bolzman machines, projection pursuit, a hidden Markov model support vector machines, kernel regression, discriminant analysis and classification, k-nearest-neighbour analysis, fuzzy neural networks, Bayesian networks, or cluster analysis.

69. A system as claimed in claim 33 wherein the predictor function is a predictor function with having minimal prediction error.

70. A system as claimed in claim 33 wherein the information comprises at least one of the pedigree of the individual; an estimated breeding value of the individual; data on genetic markers across the genome for the individual or for relatives of the individual; at least one index of phenotype for the individual or for relatives of the individual; at least one marker predictive of phenotype for the individual or for relatives of the individual; and at least one index of epigenetic modification or status for the individual, or a combination thereof.
